(19) United States
(12) Patent Application Publication
(10) Pub. No.: US 2026/0053943 A1
(43) Pub. Date: Feb. 26, 2026

(54) NANOCOMPOSITION COMPRISING ANTIBODY-DRUG CONJUGATE AND USE THEREOF

(71) Applicant: Fulgent Genetics, Inc., El Monte, CA (US)

(72) Inventors: Ray YIN, El Monte, CA (US); Jing PAN, El Monte, CA (US); Kai QI, El Monte, CA (US); Zhiying ZHOU, El Monte, CA (US); Qun SUN, El Monte, CA (US); Lin WANG, El Monte, CA (US); Xueping JIANG, El Monte, CA (US); Sichang ZHOU, El Monte, CA (US); Can MAO, El Monte, CA (US); Yuzhong CHEN, El Monte, CA (US); Jin CHU, El Monte, CA (US)

(21) Appl. No.: 19/373,945

(22) Filed: Oct. 30, 2025

Related U.S. Application Data
(63) Continuation of application No. PCT/US24/27221, filed on May 1, 2024.
(60) Provisional application No. 63/499,315, filed on May 1, 2023.
(60) Provisional application No. 63/518,879, filed on Aug. 11, 2023.
(60) Provisional application No. 63/602,649, filed on Nov. 26, 2023.

(51) Int. Cl. A61K 47/68; C07K 16/32

(52) U.S. Cl. CPC A61K 47/6851; A61K 47/68037; A61K 47/6889; C07K 16/32

(57) ABSTRACT

This disclosure is directed to a pharmaceutical composition for treating or preventing a disease. The pharmaceutical composition can comprise a targeting bioactive agent (TBA); a payload bioactive agent (PBA) covalently linked to the targeting bioactive agent (TBA) directly or indirectly; and, optionally, a polymer forming nanoaggregates. The pharmaceutical composition can comprise Ag⁺ tumor cytotoxicity to tumor cells having a tumor antigen (Ag⁺ tumor cells) and Ag⁻ tumor cytotoxicity to tumor cells free from a tumor antigen (Ag⁻ tumor cells). The pharmaceutical composition can be an antibody-drug conjugate (ADC) for treating cancers having tumor antigen positive (Ag⁺) tumor cells, tumor antigen negative (Ag⁻) tumor cells, or heterogenous cancers having both tumor antigen positive (Ag⁺) tumor cells and tumor antigen negative (Ag⁻) tumor cells.

### CROSS REFERENCE

The application is a continuation of International Application No. PCT/US2024/027221, filed May 1, 2024, which claims the benefit of U.S. Application No. 63/499,315, filed on May 1, 2023, U.S. Application No. 63/518,879, filed Aug. 11, 2023, and U.S. Application No. 63/602,649, filed on Nov. 26, 2023, all of which are hereby incorporated by reference in their entireties.

### FIELD OF THE DISCLOSURE

The present disclosure relates to a pharmaceutical composition that can be used for treatment of a disease in patients in need thereof. The pharmaceutical composition can comprise nanoaggregates formed from a polymer, a targeting bioactive agent (TBA), a cytotoxic bioactive agent (also referred to as “payload bioactive agent”, PBA).

### BACKGROUND

Antibody-drug conjugates (ADCs) are one of the fastest growing drug classes in oncology therapy. ADCs are composed of monoclonal antibodies (mAbs) linked to cytotoxic drugs and are designed to improve the therapeutic efficacy of antineoplastic agents by targeted delivery to cells that express the specific target molecules, such as target antigen of interest. ADCs have shown promising results in treating cancers, with multiple drugs approved by the US Food and Drug Administration (FDA) and dozens more currently in preclinical and clinical development. Some approved or to be approved drugs can include Gemtuzumab ozogamicin (Mylotarg, Pfizer), an anti-CD33 mAb-calicheamicin conjugate; brentuximab vedotin (Adcetris, Seagen/Takeda), a CD30-specific mAb linked to monomethyl auristatin E (MMAE); polatuzumab vedotin (Polivy, Roche); belantamab mafodotin (Blenrep, GlaxoSmithKline); Trastuzumab emtansine (T-DM1; Kadcyla, Roche) for any solid tumour, for early-stage and metastatic, HER2⁺ breast cancer; trastuzumab deruxtecan (Enhertu, Daiichi Sankyo/AstraZeneca), a second HER2⁻ targeted ADC; sacituzumab govitecan (Trodelvy, Gilead Sciences), an anti-TROP2 mAb conjugated to SN-38, the active metabolite of irinotecan, for previously treated metastatic triple-negative breast cancer; Enfortumab vedotin (Padcev, Seagen/Astellas Pharma), an MMAE-conjugated nectin-4-directed mAb for the urothelial cancer market; and the latest cetuximab sarotalocan (Akalux, Rakuten Medical), an epidermal growth factor receptor (EGFR)-targeted mAb conjugated to a dye that induces tumour cell death upon photoactivation. Typical mechanisms of targeted delivery by ADC can involve receptor-mediated endocytosis. For example, the anti-tumor activity of the ADC ENHERTU® (fam-trastuzumab-deruxtecan-nxki) is mainly achieved by the binding of the human epidermal receptor 2 (HER2) by monoclonal antibody trastuzumab, and delivery of cytotoxic drug deruxtecan via antibody-dependent receptor-mediated endocytosis (RME). The anti-tumor activity of the ADCs requires the tumor cells having specific tumor antigens tha can be specifically targeted by the antibody, such as the human epidermal receptor 2 (HER2) in HER2-positive breast cancer.

However, the design of clinically effective antibody-drug conjugates (ADCs), has presented challenges for the industry: since the first ADC, Mylotarg® (gemtuzumab ozogamicin), was approved in 2000 by the US Food and Drug Administration (FDA), there have been only 14 ADCs received market approval so far worldwide after over 30 years of research and development. Over 100 ADC candidates are still being investigated in clinical stages at present.

Recently, it has been proposed to couple antibodies on the surface of nanoparticles containing drug that can specifically bind to target cells and deliver the drug payload (Alberto Juan, et a., Int J Mol Sci. 2020 September; 21(17): 6018, Aug. 21, 2020. doi: 10.3390/ijms21176018). However, the mechanism of such delivery is still unknown.

There is a continued need for new ways that can deliver drugs more effectively or to improve upon the tissue specificity and cytotoxicity.

### SUMMARY

The present disclosure is directed to a pharmaceutical composition comprising: a targeting bioactive agent (TBA) comprising at least a targeting moiety having binding affinity to a target molecule or a target cell; a payload bioactive agent (PBA) covalently linked to the TBA directly or indirectly; a polymer comprising at least one first terminal group and at least one second terminal group; and optionally a pharmaceutical suitable carrier; wherein the polymer is covalently or non-covalently bond to the TBA, the PBA, or a combination thereof; and wherein the polymer comprises polyoxazoline (POX), a polyethylene glycol (PEG), or a combination thereof, wherein the polyoxazoline (POX), when present, comprises a linear portion, a branched portion, or a combination thereof, and wherein the polyoxazoline (POX) comprises poly(2-methyloxazoline), poly(2-ethyloxazoline), poly(2-propyloxazoline), poly(2-isopropyloxazoline), poly(2-substituted oxazoline), or a combination thereof.

The present disclosure is also directed to an antibody-drug conjugate (ADC) comprising: a targeting bioactive agent (TBA) comprising an antibody or a part thereof having binding affinity to a target molecule that comprises a tumor antigen (Ag); a payload bioactive agent (PBA) covalently linked to the targeting bioactive agent (TBA) directly or indirectly; a polymer; and the ADC having a formula selected from Formula F2 to Formula F27 disclosed herein, an isomer thereof, an isotope derivative thereof, and a combination thereof, wherein, m is an integer in a range of from 1-100, m′ is an integer in a range of from 1-100, and n is an integer in a range of from 1 to 100.

The present disclosure is further directed to a method for treating or preventing a disease of a subject in need thereof, the method comprising administering the subject with an effective dose of the pharmaceutical composition disclosed herein.

### BRIEF DESCRIPTION OF THE FIGURES

FIG. 1A-FIG. 1N. Schematic illustrations of representative examples of the pharmaceutical composition having nanoaggregates comprising a targeting bioactive agent (TBA), a payload bioactive agent (PBA) and a polymer. FIG. 1A: An example of pharmaceutical composition and nanoaggregates that comprise no cleavable linker between the targeting bioactive agent (TBA) and payload bioactive agent (PBA). FIG. 1B: An example of the pharmaceutical composition and nanoaggregates that comprise at least one cleavable linker between the targeting bioactive agent (TBA) and payload bioactive agent (PBA). FIG. 1C: An example of the pharmaceutical composition and nanoaggregates that comprise a polymer, at least one cleavable linker between the targeting bioactive agent (TBA) and payload bioactive agent (PBA). FIG. 1D: Schematic illustration of a representative example of nanoparticle complex that can comprise multiple nanoaggregates. FIG. 1E: An example of the pharmaceutical composition comprises no cleavable linker between the targeting bioactive agent (TBA) and payload bioactive agent (PBA) with schematic illustrations of representative examples of the pharmaceutical composition having the polymers non-covalently associated targeting bioactive agent (TBA) and payload bioactive agent (PBA). FIG. 1F: An example of the pharmaceutical composition comprises at least one cleavable linker between the targeting bioactive agent (TBA) and payload bioactive agent (PBA). FIG. 1G: A schematic illustration of a representative example of an ADC having a branched polymer side chain attached to the polymer that links the antibody and the PBA and the polymer side chain is free from any PBAs. FIG. 1H: A schematic illustration of a representative example of an ADC having a branched polymer side chain attached to the antibody and the polymer side chain is free from any PBAs. FIG. 1I: A schematic illustration of a representative example of an ADC comprising a cleavable linker and having a branched polymer side chain attached to the polymer that links the antibody and the PBA and the polymer side chain is free from any PBAs. FIG. 1J: A schematic illustration of a representative example of an ADC comprising a cleavable linker and having a branched polymer side chain attached to the antibody and the polymer side chain is free from any PBAs. FIG. 1K: A schematic illustration of a representative example of an ADC having a linear polymer side chain attached to the polymer that links the antibody and the PBA and the polymer side chain is free from any PBAs. FIG. 1L: A schematic illustration of a representative example of an ADC having a linear polymer side chain attached to the antibody and the polymer side chain is free from any PBAs. FIG. 1M: A schematic illustration of a representative example of an ADC comprising a cleavable linker and having a linear polymer side chain attached to the polymer that links the antibody and the PBA and the polymer side chain is free from any PBAs. FIG. 1N: A schematic illustration of a representative example of an ADC comprising a cleavable linker and having a linear polymer side chain attached to the antibody and the polymer side chain is free from any PBAs. R1 and R2 represent the first terminal group and the second terminal group, respectively.

FIG. 2A-FIG. 2E. Schematic illustrations of representative examples of a pharmaceutical composition having the polymers covalently linked to the targeting bioactive agent (TBA) and payload bioactive agent (PBA). FIG. 2A: An example of a pharmaceutical composition comprises no cleavable linker. FIG. 2B: An example of the pharmaceutical composition comprises at least one cleavable linker in the linker L1 between the TBA and the polymer. FIG. 2C: An example of the pharmaceutical composition comprises at least one cleavable linker in the linker L2 between the PBA and the polymer. FIG. 2D: An example of the pharmaceutical composition comprises at least one cleavable linker in the linker L1 between the TBA and the polymer and at least one cleavable linker in the linker L2 between the PBA and the polymer. FIG. 2E: An example of the pharmaceutical composition comprises at least two same or different PBAs. The (R1) and (R2) represent the reacted first terminal group and the reacted second terminal group, respectively. Open rectangle: linker L1. Hatched rectangle: linker L2. Solid rectangle: the polymer of this disclosure. Crossed circle: cleavable bond. Solid circle: payload bioactive agent (PBA). Each TBA can be covalently linked to one or more PBAs, n is an integer in a range of from 1 to 100.

FIG. 3A-FIG. 3D. Schematic illustrations of representative examples of the pharmaceutical composition comprising the polymers having asymmetric branches covalently linked to one TBA and multiple PBAs. FIG. 3A: An example of the pharmaceutical composition comprises no cleavable linker. FIG. 3B: An example of the pharmaceutical composition comprises at least one cleavable linker in the linker L1 between the TBA and the polymer. FIG. 3C: An example of the pharmaceutical composition comprises more than one cleavable linkers in the linker L2 between the PBA and the polymer. FIG. 3D: An example of the pharmaceutical composition comprises at least one cleavable linker in the linker L1 between the TBA and the polymer and more than one cleavable linkers in the linker L2 between the PBA and the polymer.

FIG. 4A-FIG. 4D. Schematic illustrations of representative examples of the pharmaceutical composition comprising the polymers having symmetric branches covalently linked to one TBA and multiple PBAs. FIG. 4A: An example of the pharmaceutical composition comprises no cleavable linker. FIG. 4B: An example of the pharmaceutical composition comprises at least one cleavable linker in the linker L1 between the TBA and the polymer. FIG. 4C: An example of the pharmaceutical composition comprises more than one cleavable linkers in the linker L2 between the PBA and the polymer. FIG. 4D: An example of the pharmaceutical composition comprises at least one cleavable linker in the linker L1 between the TBA and the polymer and more than one cleavable linkers in the linker L2 between the PBA and the polymer.

FIG. 5. Schematic illustration of representative examples of the formation of polymer-PBA aggregates. The pharmaceutical composition can comprise at least a cleavable linker in linker L1.

FIG. 6. Schematic illustration of representative examples of the formation of polymer-PBA aggregates. The pharmaceutical composition can comprise cleavable linkers in both linkers L1 and L2.

FIG. 7. A representative example of a reaction scheme for the production of polymer having disulfide bonds and covalent linkage of multiple drug molecules of exatecan (Exa) to the polymer.

FIG. 8. A representative example of a reaction scheme for the production of an antibody-drug conjugate (ADC) through one or more lysine residues of the antibody. Multiple molecules of the drug Exa can be linked to the TBA.

FIG. 9. A representative example of a reaction scheme for the production of antibody-drug conjugate (ADC) through one or more cysteine residues of the antibody. Multiple molecules of the drug Exa can be linked to the TBA.

FIG. 10A-FIG. 10B. Representative examples of antibody-drug conjugates (ADCs) having at least one cleavable linker GGFG and drug molecule as a PBA. FIG. 10A: An example of ADC formed through one or more lysine residues of the antibody. FIG. 10B: An example of ADC formed through one or more cysteine residues of the antibody.

FIG. 11A-FIG. 11B. Representative examples of antibody-drug conjugates (ADCs) having branched polymer and the cleavable linker GGFG. FIG. 11A: An example of ADC formed through one or more lysine residues of the antibody with multiple drug PBA molecules covalently linked to a branched polymer. FIG. 11B: An example of ADC formed through one or more cysteine residues of the antibody with multiple drug PBA molecules covalently linked to a branched polymer.

FIG. 12. Representative examples of reaction scheme of end group functionalization via H-initiated PEOX. INT-1: An example of a compound having a -PA functional group. INT-2: An example of a compound having a —NH₂ functional group. INT-3: An example of a compound having an —OH functional group. INT-4: An example of a compound having a —N₃ functional group. INT-5: another example of a compound. INT-6: Yet another example of a compound. INT-7: Yet another example of a compound. INT: intermediate.

FIG. 13A-FIG. 13C. Representative examples of reaction scheme of end group functionalization via H-initiated PEOX for multiple drug molecules (PBAs). FIG. 13A: An example of a polymer having two PBAs (INT-8) and an example of a polymer having 4 PBAs (Drugs) (INT-9). FIG. 13B: Another example of a polymer having two PBAs (INT-10). FIG. 13C: Another example of a polymer having 4 PBAs (Drugs) (INT-11).

FIG. 14. Representative examples of preparation of heterofunctional PEOX having multiple payload bioactive agents (PBAs) having linker L2. INT-12: An example of a polymer having two PBAs. INT-13: An example of a polymer having 4 PBAs.

FIG. 15A-FIG. 15B. Representative examples of preparation of heterofunctional PEOX for the formation of ADCs having linkers L1 and L2. FIG. 15A: An example of preparation of an ADC having two PBAs. FIG. 15B: An example of preparation of an ADC having 4 PBAs.

FIG. 16. Representative examples of preparation of linker-PEOX-PBAs for the formation of ADCs having at least two PBAs covalently linked to each PEOX polymer with at least a linker positioned between the TBA and the PEOX polymer. One or more linker-PEOX-PBAs can be attached to each antibody.

FIG. 17. Representative examples of preparation of linker-PEOX-PBAs for the formation of ADCs having at least four PBAs covalently linked to each PEOX polymer with at least a linker positioned between the TBA and the PEOX polymer. One or more linker-PEOX-PBAs can be attached to each antibody.

FIG. 18A-FIG. 18J. Representative examples of antibody-drug conjugates (ADCs). FIG. 18A: Formula F10. FIG. 18B: Formula F11. FIG. 18C: Formula F12. FIG. 18D: Formula F13. FIG. 18E: Formula F14. FIG. 18F: Formula F15. FIG. 18G: Formula F16. FIG. 18H: Formula F17. FIG. 18I: Formula F18. FIG. 18J: Formula F19.

FIG. 19A-FIG. 19B. Representative examples of antibody-drug conjugate (ADC) having polymer and cleavable linkers. FIG. 19A: Formula F20 with a linker. FIG. 19B: Formula F21 with a linker.

FIG. 20A-FIG. 20B. Representative examples of antibody-drug conjugate (ADC) having polyethyleneglycol (PEG) and cleavable linkers. FIG. 20A: Formula F22 with cleavable linker. FIG. 20B: Formula F23 with cleavable linker. m=1 to 100. n=1 to 100.

FIG. 21A-FIG. 21B. Representative examples of antibody-drug conjugate (ADC) having polymer and cleavable linkers. FIG. 21A: Formula F24 with multiple cleavable linkers and payload bioactive agents (PBAs). FIG. 21B: Formula F25 with multiple cleavable linkers and PBAs. n=1-100.

FIG. 22A-FIG. 22B. Representative examples of antibody-drug conjugate (ADC) having polyethyleneglycol (PEG) and cleavable linkers. FIG. 22A: Formula F26 with multiple cleavable linkers GGFG. FIG. 22B: Formula F27 with multiple cleavable linkers GGFG. m=1 to 100 and n=1-100.

FIG. 23. Representative examples of modified exatecans. Formula F30: An example of a succinic anhydride modified exatecan. Formula F31: An example of an epoxy modified exatecan, Formula F32: An example of an ethylene carbonate modified exatecan.

FIG. 24. Representative examples of modified exatecans. Formula F33: An example of a 2-thiirane methanol or a derivative modified exatecan. Formula F34: An example of a 1,3-oxathiolan-2-one modified exatecan. Formula F35: An example of 1,3-Oxathiolane-2-thione modified exatecan. Formula F36: An example of an ethylene trithiocarbonate modified exatecan.

FIG. 25A-FIG. 25B. Representative examples of modified exatecans. FIG. 25A: An example of an exatecan modified with succinic anhydride and HOCH₂CH₂NH₂. FIG. 25B: A formula of deruxtecan.

FIG. 26A-FIG. 26D. Representative examples of modified exatecans. FIG. 26A: An example of exatecan having a hydrogen as an end group. FIG. 26B: An example of exatecan having an N₃ group as an end group. FIG. 26C: An example of exatecan having an —NHBoc as an end group. FIG. 26D: An example of exatecan having an —NHFMoc as an end group.

FIG. 27. Representative examples of nanoparticle size measurements. Herceptin: HERCEPTIN® (Trastuzumab), a HER2 monoclonal antibody. Enhertu: ENHERTU® (fam-trastuzumab-deruxtecan-nxki), HER2-deruxtecan ADC comprising monoclonal antibody Trastuzumab and cytotoxic drug deruxtecan. Kanjinti: Herceptin biosimilar known as a brand name KANJINTI® (trastuzumab-anns), and an unlimited example of a Nano-ADC (Example 1) from the instant disclosure.

FIG. 28A-FIG. 28C. Representative examples of cytotoxicity measured using in vitro cell assay. FIG. 28A: Cytotoxicity assay using MDA-MB231 cells, a triple-negative breast cancer (TNBC) cell line. FIG. 28B: Cytotoxicity assay using HT-29 cells, a colon cancer cell line. FIG. 28C: Cytotoxicity assay using HCT-116 cells, a colon cancer cell line. Enhertu: ENHERTU® (fam-trastuzumab-deruxtecan-nxki) and a Nano-ADC (Example 1) from the instant disclosure.

FIG. 29A-FIG. 29B. Representative examples of cytotoxicity in non-tumor cells and tumor cells. FIG. 29A: Enhertu cytotoxicity. FIG. 29B: Differential cytotoxicity of a Nano-ADC (Example 1) of this disclosure. HDFa: Primary Dermal Fibroblast cells from normal human. HFC: epithelial cells from normal human. SK-BR-3: a human breast cancer cell line that overexpresses the Her2 protein. NCI-N87: a gastric cancer cell line that overexpresses Her2 protein.

FIG. 30. Schematic illustrations of tumor cell cytotoxicity mechanism models. The models include: traditional receptor-mediated endocytosis (RME); a representative example of Nanoparticle-mediated cytotoxicity (NMC) to deliver Ag⁺ tumor cytotoxicity to tumor cells having a tumor antigen (Ag⁺ tumor cells) and Ag⁻ tumor cytotoxicity to tumor cells free from any tumor antigen (Ag⁻ tumor cells) and a representative example of Nanoparticle-mediated immune modification (NMIM) to enhance immune system response to delivery Ag⁺ tumor cytotoxicity to Ag⁺ tumor cells and Ag⁻ tumor Ag⁻ tumor cells.

### DETAILED DESCRIPTION

Features and advantages of the present disclosure will be more readily understood, by those of ordinary skill in the art, from reading the following detailed description. It is to be appreciated that certain features of the disclosure, which are described above and below in the context of separate embodiments, may also be provided in combination in a single embodiment. Conversely, various features of the disclosure that are, for brevity, described in the context of a single embodiment, may also be provided separately or in any combination or sub-combination. In addition, references in the singular may also include the plural (for example, “a” and “an” may refer to one, or one or more) unless the context specifically states otherwise.

Use of numerical values in the various ranges specified in this application, unless expressly indicated otherwise, are stated as approximations as though minimum and maximum values within the stated ranges were both proceeded by the word, “about”. In this manner, slight variations above and below the stated ranges can be used to achieve substantially the same results as values within the ranges. Also, disclosure of ranges is intended as a continuous range including every value between the minimum and maximum values and including the minimum and maximum cited values.

The drug solubility in the instant disclosure is defined as, relative to parts of solvent required to solubilize one part of bioactive agent or drug, <30 (soluble), 30-100 (poorly soluble) and >100 (insoluble). Water solubility is defined herein as, relative to parts of water required to solubilize one part of bioactive agent or drug, <30 (water soluble), 30-100 (poorly water soluble) and >100 (water insoluble).

The term “polymer” refers to any polymer suitable for this disclosure as defined above and hereafter. In examples, a polymer can comprise polyethylene glycol (PEG), polyoxazoline or modified polyoxazoline as disclosed herein. In further examples, the polymer can comprise a modified polyoxazoline can comprise one or more second terminal groups, such as an —NH₂, —NH, —NH₃⁺, other basic groups or a combination thereof, with a proviso that in a range of from 0.01% to 100% of the second terminal group is free from primary amine. In some cases, in a range of from 0.010% to 100%, 0.10% to 100%, or 1% to 100%, of the second terminal group is free from primary amine. In some cases, in a range of from 1% to 100% of the second terminal group can comprise hydroxyl group. All percentages are based on the total number of the second terminal groups. The polymer can comprise a linear polymer, a branched polymer, a symmetrically branched polymer, an asymmetrically branched polymer, a dendrimer, a dendrigraft polymer, a comb-branched polymer, a star-branched polymer, or a combination thereof. The polymer is water soluble. In examples, the polymer can be dissolved in water to produce a 12% weight percent or higher water solution.

The term “bioactive agent” or “bioactive agents” refers to a molecule, a compound, a complex of one or more compounds or molecules, or a combination thereof that can provide a biological activity in vivo, in vitro, or a combination thereof. A pharmaceutical composition can comprise one or more bioactive agent such as pharmaceutically active agents (PAAs) or active pharmaceutical ingredients (APIs), and other bioactive or inert compounds that can include emollients, bleaching agents, antiperspirants, pharmaceuticals, moisturizers, scents, colorants, pigments, dyes, antioxidants, oils, fatty acids, lipids, inorganic salts, organic molecules, opacifiers, vitamins, pharmaceuticals, keratolytic agents, UV blocking agents, tanning accelerators, depigmenting agents, deodorants, perfumes, insect repellants, or a combination thereof. Some examples of bioactive agents are described in detail in this disclosure.

The term “targeting bioactive agent (TBA)” refers to a compound or a molecule that can comprise at least a targeting moiety having binding affinity to a target molecule or a target cell. In some cases, a targeting bioactive agent (TBA) can comprise peptide, a protein, DNA, RNA, nucleic acids, oligo nucleic acids, mRNA, siRNA, sgRNA, a monoclonal antibody, a fragment of a monoclonal antibody, a polyclonal antibody, a fragment of a polyclonal antibody, a synthetic antibody, a fragment of a synthetic antibody, antigen-binding portions thereof, a bispecific antibody, a single-chain antibody, a single-chain fragment variable (scFv) that contains the complete antigen-binding domains of a whole antibody, a ligand of cell surface receptor, an agonist, an activator, an inhibitor, an antagonist, a naked nucleic acid, a nucleic acid, a ribozyme, a virus, a virus-like particles and a combination thereof, Alemtuzumab, alemtuzumab, an antisense nucleic acid, anti-CD19 antibodies, anti-CD20 antibodies, anti-CTLA-4 (cytotoxic T lymphocyte-associated antigen) antibodies, anti-LAG3 (lymphocyte activation gene-3) antibodies, anti-PD1 antibodies KEYTRUDA® (pembrolizumab), anti-PD-L1 antibodies, anti-TIM-3 (T cell immunoglobulin and mucin domain-3) antibodies, Atezolizumab, BAVENCIO® (avelumab), Bevacizumab, bevacizumab, Blinatumomab, Brentuximab, Cetuximab, cetuximab, chimeric antigen receptor or chimeric antigen T-cell receptor (CAR-T), cytokines, Daratumumab, Denosumab, Dinutuximab, Durvalumab, Elotuzumab, gemtuzumab, granulocyte colony stimulating factor (G-CSF), Ibritumomab, IMFINZI® (durvalumab), Inotuzumab, interferon α, interferon α2a, interleukins, Ipilimumab, lectins, Necitumumab, Neupogen (Filgrastim), Obinutuzumab, Ofatumumab, Olaratumab, OPDIVO® (nivolumab), Panitumumab, Pembrolizumab, Pertuzumab (anti-HER2), Ramucirumab, Rituximab, rituximab, Siltuximab, toripalimab, Tislelizumab (BGB-A317), siRNAs, T-cell receptor (TCR), TECENTRIQ® (atezolizumab), tositumomab, trastuzumab (anti-HER2), and a combination thereof. In some cases, a TBA can comprise a binding affinity to one or more of the target molecules disclosed hereafter.

The term “target molecule”, “target antigen”, “target antigens”, “targeting molecule” or “targeting molecules”, collectively “target molecule” or “target molecules”, refers to a peptide, a protein, DNA, RNA, nucleic acids, oligo nucleic acids, mRNA, siRNA, sgRNA, an antibody, a fragment of an antibody, a cell surface protein (either in cell bound form or a cell free form), a cell surface receptor (either in cell bound form or a cell free form), a ligand of cell surface receptor, a gene, a ribozyme, a cell, a virus, a virus-like particles, or a combination thereof that can have affinity to the targeting bioactive agent (TBA). In some cases, a target molecule can comprise a gene or a gene product selected from PD-1, PD-L1, 4-1BB, 5T4 (5T4 oncotrophoblast glycoprotein), ABL1, ABL2, ACVR1, AKR1C3, AKT, ALK, ASCL1, ASNS, Asparagine, ASGR1, ASGPR1, ATM, ATR, ATRX, AURKA, AURKB, AXL, B7H3 (CD276 antigen), B7H4 (V-set domain-containing T-cell activation inhibitor 1), B7H7 (HHLA2), BAK1, BAX, BCL2 family members (BCL2, BCL2L1, MCL1, BCL2A1, BAK1, BAX), BCL2A1 (BFL1), BCMA (TNFRSF17), BCOR, BET bromodomain family, BIRC5 (survivin), BMPR, BRAF, BRD1, BRD4, CCND1, CCND2, CCR8, CD19, CD22, CD25, CD274 (PD-L1), CD276 (B7-H3), CD3, CD16a, CD28, CD33, CD37, CD38, CD40, CD40L, CD47, CD52, CD7, CD70, CD73, CD79B, CD200R1 (Cell surface glycoprotein OX2 receptor 1), CDC7, CDK12, CDK2, CDK4, CDK6, CDK7, CDK9, CHEK1 (CHK1), CK1 (casein Kinase 1), CK2 (casein kinase 2), CLDN18.2, CRBN [cereblon E3 ubiquitin ligase], CREBBP/EP300, CRTAM, CSF1R, CSF2 (GM-CSF), CTAGIB (NY-ESO-1), CTLA4, CTNNB1, CXCL10, CXCR4, DDX3X, DLK1, DLL3 (Delta-like Ligand 3), DNA (alkylators), DNA-PK, DNMT (DNA methyl transferase), DOT1L, EED, EGFR, EGFRvIII (EGFR variant III), EpCAM (TROP1), EPHA2, ERBB2 (HER2), ETS gene fusions, EWSR1-FLI1, EZH2, F3 (tissue factor), FGFR, FLT3, FOLR1 (folate receptor alpha), Gamma secretase, GD2 (disialoganglioside), GFI1, GFI1B, GPC2, GPC3, GPNMB, GSK3, HAVCR2 (TIM3), TIMD4 (TIM4), HDAC, HIF1A, Hippo pathway (YAP1, WWTR1 (TAZ), TEADs), Histone H3, HSP90, IDH1, IDH2, IDO1, IFNG (interferon gamma), IGF1R, IL13RA2, IL2, IL3RA (CD123), IL6, IL23p19, Inhibitor of apoptosis (IAP) proteins, JAK1, JAK2, JAK3, KAT6A (MYST3), KDM1A (LSD1), KDM4A, KIT, KMT2A (MLL), KMT2E (MLL5), LAG3, LIFR, LIN28B, LRRC15, MAGEA3, MAP2K1 (MEK1), MAP2K2 (MEK2), MAPK3 (ERK1), MAPK1 (ERK2), MCL1, MDM2, MEN1 (menin), MET, MGMT, MS4A1 (CD20), MSLN (mesothelin), MTOR, mTORC1, mTORC2, MYC, MYCN, NAMPT, NCAM1 (CD56), MUC1, MUC16, NEDD8 activating enzyme (NAE), Neoantigens, NF-kappa-B, NKp30, NKp46, NOTCHI, NR5A1 (steroidogenic factor 1), NT5C2, NTRK, NUTM1 gene fusions, ODC1, OLIG2, PARP, PAX5, PAX-FOXO1, PDCD1 (PD-1), PD-L1, PDGFRA, PDGFRB, PDPK1 (3-phosphoinositide dependent protein kinase 1), PIK3CA (P1I3K-alpha), PIK3CD (PI3K-delta), PIM1, PKA (protein kinase A), PKC (protein kinase C), PLK1, PML-RARA, PPM1D (WIP1), PRAME, PRDM1, PRDM10, PRDM8, PRMT2, PRMT5, Proteasome, PSMA, PTEN, PTK2 (FAK), PTPN (protein tyrosine phosphatase), PTPN11 (SHP2), RAS, RELA, RET, RIGI (DDX58), RNA polymerase (RNApol) I, ROR1, ROR2, ROS1, RPA3, SARS-Cov-2, SH2B3, SLC16A1 (MCT1), SMO, SMYD3, SSTR (somatostatin receptor), STAT2, STAT3, STEAP1, STING1 (STING), SUZ12, SWI/SNF, SYK, SYT-SSX, TERT, TET2, TGF-beta, TNFRSF4 (OX40), OX40L, TNFRSF8 (CD30), TNFSF10 (TRAIL), TOP1, TOP2 (DNA topoisomerase I/II), TROP2, TP53, TSLP, Tubulin, TYK2, TYMS, VEGF, VEGFR, WDR5, WEE1, WT1, XPO1 (Exportin 1), YAP1, ZBTB17 (MIZ-1), or a combination thereof.

The term “payload bioactive agent (PBA)” refers to a natural or synthetic small molecule-based drug, inorganic-based drug, a toxin molecule having cytotoxicity, a molecule having cytotoxicity to target cells, a derivative thereof, or a combination thereof. The term “PBA” and the term “drug” may be used interchangeably, i.e., where a term “drug” or “Drug” is shown in texts or drawings, it may refer to “PBA” or a specific drug. In some cases, a payload bioactive agent (PBA) can be selected from 5-FU (Fluorouracil), Abemaciclib, Abiraterone Acetate, Acalabrutinib, Afatinib Dimaleate, Aldesleukin (Il-2 Or Interleukin-2), Alectinib Hydrochloride, Altretamine, Amifostine, Aminolevulinic Acid Hydrochloride, Anastrozole, Apremilast, Aprepitant, Arsenic Trioxide, Asparaginase Erwinia Chrysanthemi, Axicabtagene Ciloleucel, Axitinib, Azacitidine, Belinostat, Bendamustine Hydrochloride, Bexarotene, Bicalutamide, Bleomycin Sulfate, Bortezomib, Bosutinib Monohydrate, Brigatinib, Busulfan, Cabazitaxel, Cabozantinib S-Malate, Capecitabine, Carboplatin, Carfilzomib, Carmustine, Carmustine Implant, Ceritinib, Chlorambucil, Cisplatin, Cladribine, Clofarabine, Cobimetinib Fumarate, Copanlisib Dihydrochloride, Crizotinib, Cyclophosphamide, Cytarabine, Cytarabine Liposome, Cytarabine; Daunorubicin, Dabrafenib Mesylate, Dacarbazine, Dactinomycin, Dasatinib, Daunorubicin Citrate, Daunorubicin Hydrochloride, Decitabine, Defibrotide Sodium, Degarelix Acetate, Denileukin Diftitox, Dexamethasone, Docetaxel, Doxorubicin Hydrochloride, Edoxaban Tosylate, Eltrombopag Olamine, Enasidenib Mesylate, Enzalutamide, Epirubicin Hydrochloride, Eribulin Mesylate, Erlotinib Hydrochloride, Estramustine Phosphate Sodium, Etoposide, Everolimus, Exemestane, Exatecan, Dxd (Deruxtecan, A Derivative Of Exatecan), Filgrastim, Floxuridine, Fludarabine Phosphate, Fluorouracil, Flutamide, Fulvestrant, Gefitinib, Gemcitabine Hydrochloride, Glucarpidase, Goserelin Acetate, Hydroxyurea, Ibrutinib, Idarubicin Hydrochloride, Idelalisib, Ifosfamide; Mesna, Imatinib Mesylate, Imiquimod, Irinotecan Hydrochloride, Ixabepilone, Ixazomib Citrate, Lanreotide Acetate, Lapatinib Ditosylate, Lenalidomide, Lenvatinib Mesylate, Letrozole, Leucovorin Calcium, Leuprolide Acetate, Lomustine, Mechlorethamine Hydrochloride, Megestrol Acetate, Melphalan, Mercaptopurine, Mesna, Methotrexate, Methotrexate Sodium, Methylnaltrexone Bromide, Midostaurin, Mitomycin, Mitotane, Mitoxantrone Hydrochloride, Naldemedine Tosylate, Nelarabine, Neratinib Maleate, Netupitant; Palonosetron Hydrochloride, Nilotinib Hydrochloride Monohydrate, Nilutamide, Niraparib Tosylate, Olaparib, Omacetaxine Mepesuccinate, Ondansetron, Ondansetron Hydrochloride, Osimertinib Mesylate, Oxaliplatin, Paclitaxel, Palbociclib, Palifermin, Palonosetron Hydrochloride, Pamidronate Disodium, Panobinostat Lactate, Pazopanib Hydrochloride, Pegademase Bovine, Pegaspargase, Pegfilgrastim, Peginterferon Alfa-2b, Pemetrexed Disodium, Pentostatin, Plerixafor, Plicamycin, Pomalidomide, Ponatinib Hydrochloride, Pralatrexate, Prednisone, Procarbazine Hydrochloride, Propranolol Hydrochloride, Radium 223 Dichloride, Raloxifene Hydrochloride, Rasburicase, Regorafenib, Rolapitant Hydrochloride, Romidepsin, Romiplostim, Rucaparib Camsylate, Ruxolitinib Phosphate, Sipuleucel-T, Sonidegib Phosphate, Sorafenib Tosylate, Streptozocin, Sunitinib Malate, Talc, Talimogene Laherparepvec, Tamoxifen Citrate, Temozolomide, Temsirolimus, Teniposide, Thalidomide, Thioguanine, Thiotepa, Tisagenlecleucel, Topotecan Hydrochloride, Toremifene Citrate, Trabectedin, Trametinib Dimethyl Sulfoxide, Uridine Triacetate, Valrubicin, Vandetanib, Vemurafenib, Venetoclax, Vinblastine Sulfate, Vincristine Sulfate, Vinorelbine Tartrate, Vismodegib, Vorinostat, Ziv-Aflibercept, Zoledronic Acid, or a combination thereof. In some cases, a cytotoxic compound or molecule can be preferred. In some cases, a toxin, such as ricin, can be suitable.

The term “ADC” refers to antibody-drug conjugate that is typically composed of a monoclonal antibody (mAbs) covalently attached to a cytotoxic drug via a chemical linker.

In some examples, an ADC can comprise an anti-HER2 monoclonal antibody. The term “Her2”, “HER2”, “ErbB2”, or “Neu” refer to a transmembrane glycoprotein in the ErbB family of tyrosine kinase receptors for EGF superfamily growth factors. ErbB2 is widely expressed in epithelial cells and over-expressed in a large number of breast carcinomas and is considered as a tumor antigen (Ag) present in tumor cells. ErbB2 plays roles in development, cancer, communication at the neuromuscular junction, and regulation of cell growth and differentiation.

The ErbB2/ErbB3 heterodimer is expressed in the majority of breast, skin, ovary and gastrointestinal tumors and transduces a highly mitogenic signal in response to neuregulin 1 (NRG1; heuregulin 1) or NRG2. ErbB3, ErbB2 and neuregulin are all required for formation of the sympathetic nervous system. Antibody-drug conjugates (ADCs) can potentially, via specificity of a monoclonal antibody (mAb), selectively deliver highly cytotoxic small molecules to targeted cancer cells, leading to an enhanced therapeutic index through increased antitumor activity (tumor cytotoxicity) particularly the Ag⁺ tumor cytotoxicity to tumor cells having the tumor antigen (Ag⁺ tumor cells) and decreased off-target toxicity, i.e. cytotoxicity to non-tumor cells (non-tumor cytotoxicity). Anti-HER2 ADCs trastuzumab emtansine and trastuzumab deruxtecan are examples of the ADCs for the treatment of patients with human epidermal growth factor receptor 2 (HER2)-positive breast cancer. Currently, a total of 29 ADC candidates are being investigated in different stages of clinical development for the treatment of HER2-positive breast cancer. Currently, no anti-HER2-ADCs have been reported for the treatment of HER2-negative breast cancer or other HER2-negative/HER2 low cancers. These ADCs are also referred to as “traditional ADCs” in this disclosure.

The term “Ag⁺ tumor cell” or “Ag⁺ tumor cells” refers to cells or tumors that have abnormally high levels of a specific tumor antigen (herein “Ag”). For example, HER2⁺ tumor cells can have abnormally high levels of the tumor antigen Her2 protein. About 14% of new female breast cancer cases in the United States between 2015 and 2019 are HER2⁺. Treatment for HER2⁺ cancers may include a targeted therapy with an ADC fam-trastuzumab deruxtecan (ENHERTU® under respective trademark).

The term “Ag⁻ tumor cell” or “Ag⁻ tumor cells” refers to cells or tumors that do not have abnormally high levels of the tumor antigen (Ag). For example, a breast cancer that does not have abnormally high levels of the tumor antigen Her2 protein can be considered HER2⁻. About 78% of new female breast cancer cases in the United States between 2015 and 2019 are HER2⁻. HER2-low breast cancer is a new classification that's used to describe cancer where HER2 proteins are present, but there aren't enough for the cancer to be considered HER2-positive. About 50% to 60% of breast cancers are actually HER2-low breast cancers, according to the National Cancer Institute (NCI). These HER2-low cancers have traditionally been classified as HER2-negative (HER2⁻). As used throughout this disclosure, the words HER2 and Her2 may be used interchangeably, regardless of uppercase or lower case. In this disclosure, normal cells (non-tumor cells), tumors and tumor cells without abnormally high levels of a tumor antigen (Ag) are considered Ag⁻ normal cells (normal cells) and Ag⁻ tumor cells, respectively.

Even if a cancer is considered to be HER2-negative (HER2⁻), it may still be estrogen-positive or progesterone-positive. Whether or not it's hormone-positive also affects your treatment options.

The term “Nano-ADC” refers to the nanocomposition comprising an antibody-drug conjugate disclosed herein.

The term “SN-38” or “7-ethyl-10-hydroxycamptothecin” refers to a Topoisomerase 1 (herein “Top 1” or “Top I”) inhibitor and a camptothecin derivative. Irinotecan (CPT-11) is a water soluble camptothecin analog and a prodrug of SN-38. Since the discovery of camptothecin in the tree bark of Camptotheca acuminata, the development of camptothecin derivatives as anticancer agents has resulted in the approval of a few drugs, such as irinotecan for the treatment of colon or colorectal cancer, topotecan for the treatment of small cell lung cancer, ovarian, and cervical cancer, as well as antibody-drug conjugates (ADC) using SN-38 as payload. Due to its low solubility and high toxicity, currently there is no approved SN-38 drug product for chemotherapy. Studies on polymer conjugated SN-38 made by linking SN-38 with a multiarm polyethylene glycol via a glycine linker (Sapra, P., et al., Clin. Cancer Res., 14(6):1888, 2008) and polymer micelle SN-38 (Carne, A; et al., J. Drug Delivery, Vol. 2011, p. 9, 2011, doi:10.1155/2011/869027) have been reported. However, synthesis and production of those are complex. SN-38 or a derivative thereof can be a PBA for the Nano-ADC of this disclosure.

The term “immune modulator”, “immune stimulator” refers to molecules that can modulate immune systems in vivo. Some unlimited examples of immune modulators can include mTOR, mTOR inhibitor, one or more ATP-competitive mTORC1/2 inhibitors, one or more dual PI3K-mTOR inhibitors, rapamycin, STING (stimulator of interferon genes), STING inhibitors such as C-176, C-170 and C-171; STING activators, such as 3′,3′-cGAMP (3′,3′-cyclic GMP-AMP, Cyclic GMP-AMP, cGAMP); STING agonists such as SR-717 lithium, Alpha-mangostin or diABZI STING agonist (diABZI STING agonist-1, Compound 3), STING agonist-1 (G10), CF501, CF502, CF504, CF505, CF508, CF509, CF510, CF511 (Liu, et al., Cell Research, 1-19, 2022. https://doi.org/10.1038/s41422-022-00612-2), or MSA-2; STING antagonist such as SN-011 (GUN35901) or H-151. The Nano-ADC disclosed herein, when formed in an aggregated form or a nanoparticle form, can also serve as an effective immune modulator or stimulator. These immune modulators can be used either alone or in combinations with other drugs such as immune checkpoint inhibitors or others.

The term “cleavable linker” or a grammatical variation thereof refers to a linker that can be cleaved under proper conditions. As used herein, cleavable linkers can comprise three commonly used mechanism categories: 1) protease sensitive linkers, also referred to as enzymatically-cleavable peptide linkers, 2) pH sensitive linkers, also referred to as acid cleavable linkers, and 3) glutathione sensitive linkers, also referred to as Glutathione (GSH) cleavable linkers. In some cases, a cleavable linker can comprise acid cleavable linkers that can be cleaved under low pH conditions, Glutathione (GSH) cleavable linkers including disulfide linkers that can be cleaved by GSH such as in cytoplasm, Fe(II) cleavable linkers that can be cleaved by elevating levels of ferrous iron, Cathepsin cleavable linkers that can be cleaved by cathepsin such as in lysosomes, Glycosidase cleavable linkers that can be cleaved by β-glucuronidase such as in lysosomes, Phosphatase cleavable linkers that can be cleaved by phosphatase and pyrophosphates such as in lysosomes, Sulfatase cleavable linkers that can be cleaved by sulfatase such as in lysosomes, Photo-responsive cleavable linkers that can be cleaved by irradiation with NIR light (such as λ=650-900 nm), Bioorthogonal cleavable linkers that can be cleaved by the bioorthogonal cleavage pair such as Cu(I)—BTTAA/dsProc, or a combination thereof. In some cases, a cleavable linker comprises an enzymatically-cleavable peptide linker, such as Gly-Gly-L-Phe-N-[(carboxymethoxy)methyl]Glycinamid (GGFG-NCM) linker, L-Val-L-Ala-para-aminobenzyl alcohol (VAPAB-OH) linker (herein referred to as “VAPAB linker”), L-Val-L-Cit-para-aminobenzyl alcohol (VCPAB-OH) linker, glutamic acid-glycine-citrulline (EGCit) linkers, acid sensitive hydrazone linker, a linker comprising disulfides, a thioether linker, a glutathione-sensitive disulfide linker, a linker containing a sugar molecule or moiety, a glucuronide containing linker, a glycosidase cleavable linker, a phosphatase cleavable linker, an esterase cleavable linker, a hydrolysis cleavable linker, a modified monodispersed PEG linker, a modified PEO linker, a modified poly-PEG linker, a dipeptide linker, a valine-citrulline linker, a valine-alanine linker, a polypeptide linker, a glycine-glycine-phenylalanine-glycine (GGFG) linker, p-aminobenzyloxycarbonyl (PABC) linker, or a combination thereof. Various linkers comprising glycine-glycine-phenylalanine-glycine, such as Gly-Gly-L-Phe-N-[(carboxymethoxy)methyl]Glycinamid (GGFG-NCM) linker or other forms glycine-glycine-phenylalanine-glycine (GGFG) linkers, are collectively referred to as a “GGFG linker”. Various linkers comprising valine and alanine, such as the aforementioned L-Val-L-Ala-para-aminobenzyl alcohol (VAPAB-OH) linker or other forms of valine and alanine linkers, are collectively referred to as a “VAPAB linker” or “VAPAB”. In some cases, a PEOX polymer comprising disulfide bonds can also be suitable as a cleavable linker. The term “modified monodispersed PEG linker”, “modified PEO linker”, or “modified poly-PEG linker” refers to a corresponding polymer linker that has been modified to comprise one or more cleavable bounds, such as one or more —S—S— bonds or other cleavable bounds disclosed herein. The term “linker”, “cleavable linker”, “GGFG Linker”, “GGFG”, “VAPAB linker” or “VAPAB” can also be used to describe a corresponding linker in a reacted form, i.e., a linker residue that is reacted and incorporated into an ADC, in text or in a drawing. The term “polymer”, “PEG”, “PEOX” or a variation thereof, can also be used to describe a corresponding polymer in a reacted form, i.e., a polymer that is reacted and incorporated into an ADC, in text or in a drawing.

The term “pharmaceutical suitable carrier”, “pharmaceutical suitable carriers”, “pharmaceutically suitable carrier” or “pharmaceutically suitable carriers” refers to one or more inactive ingredients that are in approved drug products. Inactive ingredients listed in the database “Inactive Ingredients in Approved Drug Products” maintained and updated by US Food and Drug Administration (FDA) can be suitable. In some cases, a pharmaceutical suitable carrier can also be referred to as an excipient.

The term “subject” or “subjects” used throughout this disclosure refers to an animal, a human or a human patient. The term “animal” refers to wild animals, captured or zoo-raised animals and domesticated animals including live stocks, farm animals, pets, laboratory animals, such as horse, cattle, pig, donkey, mule, camel, goat, sheep, monkey, rabbit, dog, cat, mouse, rat, and the like. Warm-blooded animals are suitable. The term “human” refers to a human patient having one or more diseases in need of a treatment, a person having one or more medical conditions unrelated to a treatment, or a healthy person. In some cases, a subject can be a human patient or a healthy person.

The term “antibody”, “antibodies” or “fragment of an antibody” can include natural or synthetic antibodies that selectively bind to an antigen. The term includes polyclonal and monoclonal antibodies produced from animals, cells including eukaryotic or prokaryotic cells, cell free systems, or chemical synthesis. In addition to intact immunoglobulin molecules, also included in the term “antibodies” are fragments or polymers of those immunoglobulin molecules, and human or humanized versions of immunoglobulin molecules that selectively bind a target antigen. The term also includes bispecific antibodies, single-chain antibody, a single-chain fragment variables (scFv) that contains the complete antigen-binding domains of a whole antibody. The term can also include a combination of various antibodies, parts thereof or a fragment thereof, that contain antigen-binding domains or antigen affinity of a whole antibody.

The term “aqueous solution” or “aqueous solutions” used throughout this disclosure refers to a solution that comprises in a range of from 80% to 100% water, percentage based on the total non-solid weight of the aqueous solution. An aqueous solution can further comprise additional components, such as salt, acid, base, buffer, solvent, organic solvent, particles, emulsion, solids or non-solids, detergents, small molecules, large molecules, other ingredients, or a combination thereof. The term “non-solid weight” refers to the weight from solid contents after the aqueous solution is dried out, such as by removing all the water or other liquids.

As used herein, the term “isomer” or “isomers” refers to molecules that share the same chemical formula but have their atoms connected differently or arranged differently in space, including structural isomers having respective atoms bonded together in different orders, geometric isomers having atoms bonded in the same order, but differ in the configuration around the bonds, such as cis- or trans-isomers and enantiomers having the same chemical structure but differ in three-dimensional arrangements of atoms around asymmetric carbon, such that they are mirror images of one another.

The term “isotope” or “isotopes” refers to different forms of a chemical element which have the same number of protons but a different number of neutrons in their atoms. An isotope can have different physical properties but the same chemical ones. The term also includes radioactive or non-radioactive isotopes. All compounds disclosed herein can comprise one or more isotopes. Some non-limiting examples of isotopes can include deuterium (²H), tritium (³H), isotoiodine-125 (¹²⁵I) carbon-12 (¹²C), carbon-13 (¹³C), carbon-14 (¹⁴C), ¹⁴N, ¹⁵N, ¹⁶O, ¹⁷O, ¹⁸O, ³¹P, ³²P, ³²S, ³³S, ³⁴S, ³⁶S, ³⁵S and others, and a combination thereof. Throughout this disclosure, all isotopic variations of the compounds of the present disclosure, whether radioactive or not, are encompassed within the scope of the present disclosure.

In some cases, this disclosure is directed to a pharmaceutical composition comprising:
a targeting bioactive agent (TBA) comprising at least a targeting moiety having binding affinity to a target molecule or a target cell;
a payload bioactive agent (PBA) covalently linked to the targeting bioactive agent (TBA) directly or indirectly;
a polymer comprising at least one first terminal group and at least one second terminal group; and
optionally a pharmaceutical suitable carrier;
wherein the polymer is covalently or non-covalently bond to the targeting bioactive agent (TBA), the payload bioactive agent (PBA), or a combination thereof; and
wherein the polymer comprises polyoxazoline (POX), a polyethylene glycol (PEG), or a combination thereof, wherein the polyoxazoline (POX) comprises a linear portion, a branched portion, or a combination thereof, and wherein the polyoxazoline (POX) comprises poly(2-methyloxazoline), poly(2-ethyloxazoline), poly(2-propyloxazoline), poly(2-isopropyloxazoline), poly(2-substituted oxazoline), or a combination thereof.

In some cases, the pharmaceutical composition can comprise a polymer that comprises a polyoxazoline (POX). In some cases, the pharmaceutical composition can comprise a polymer that comprises a polyethylene glycol (PEG). In some cases, In some cases, the pharmaceutical composition can comprise a polymer that comprises a combination of polyethylene glycol (PEG) and polyoxazoline (POX).

In some cases, the polymer can be covalently linked to the targeting bioactive agent (TBA) and covalently linked to the payload bioactive agent (PBA), wherein the polymer can comprise at least one reacted first terminal group and at least one reacted second terminal group; and
wherein the polymer can be positioned between the targeting bioactive agent (TBA) and the payload bioactive agent (PBA) with at least one of the reacted first terminal group covalently coupled to the TBA and at least one of the reacted second terminal group covalently coupled to the PBA.

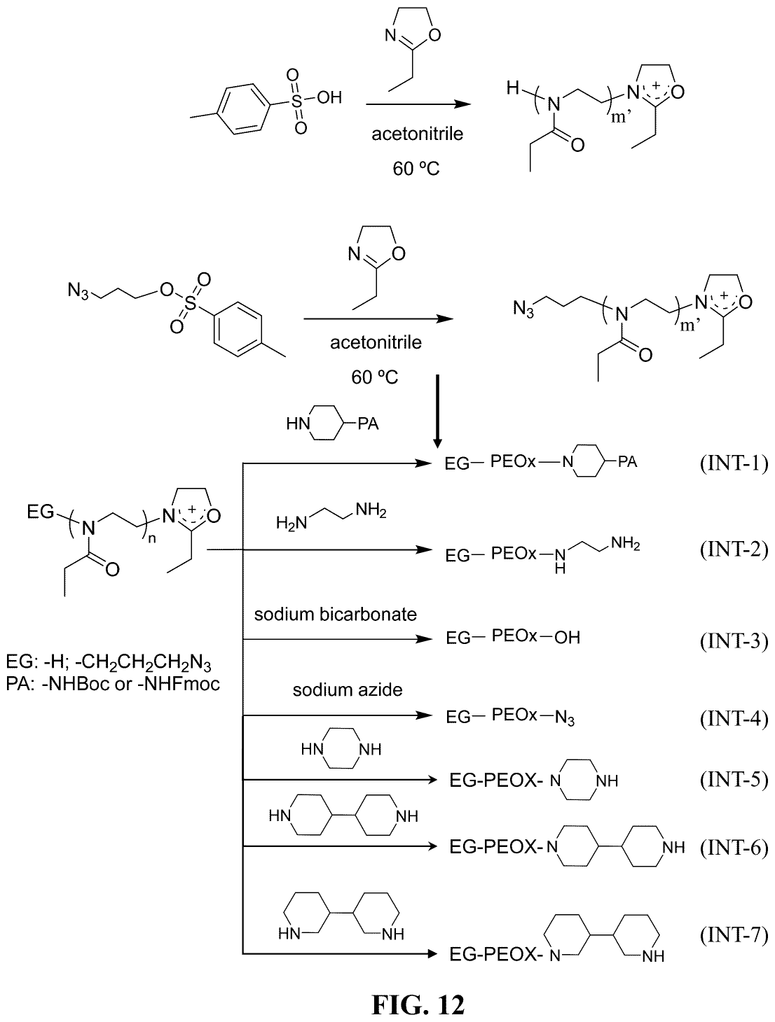

In some cases, the polymer can be covalently coupled to the TBA at the at least one reacted first terminal group via one or more coupling molecules. The term “coupled to” means covalently linked directly or via one or more coupling functional groups or molecules (collectively, “coupling molecules”). Suitable coupling molecules can include cysteine-based coupling reagents or groups, N-glycosylation based coupling reagents or groups, Boc- and Fmoc-protection and coupling reagents or groups, maleimide or SMCC based reagents or groups, and so on. In some cases, the polymer can be covalently coupled to the PBA at the at least one reacted second terminal group via one or more same or different coupling molecules. The polymer can also be functionalized with one or more functional groups. In some cases, a PEOX polymer can be functionalized to have one or more desired functional groups as the first terminal group and the second terminal group, respectively, as disclosed hereafter, such as those shown in FIG. 12 as intermediates 1-7 (herein referred to as INT-1, INT-2, INT-3, INT-4, INT-5, INT-6, and INT-7).

In some cases, the pharmaceutical composition can comprise a polymer that comprises a polyethylene glycol (PEG) covalently linked to the targeting bioactive agent (TBA) at the at least one reacted first terminal group and covalently linked to the payload bioactive agent (PBA) at the at least one reacted second terminal group, wherein the polyethylene glycol (PEG) comprises PEG monomer units in a range of from 1 to 100.

In some cases, the pharmaceutical composition can comprise a polymer that comprises a polyoxazoline (POX) covalently linked to the targeting bioactive agent (TBA) at the at least one reacted first terminal group and covalently linked to the payload bioactive agent (PBA) at the at least one reacted second terminal group, the polyoxazoline (POX) comprises a linear portion, a branched portion, or a combination thereof, and wherein the polyoxazoline (POX) comprises poly(2-methyloxazoline), poly(2-ethyloxazoline), poly(2-propyloxazoline), poly(isopropyloxazoline), or a combination thereof. The polyoxazoline (POX) can comprise oxazoline monomer units in a range of from 1 to 100.

In some cases, the payload bioactive agent (PBA) can be covalently linked to the targeting bioactive agent (TBA) indirectly via a cleavable linker comprising a cleavable bond that is cleavable in vivo or in the target cell, and wherein the cleavable linker can comprise an enzymatically-cleavable peptide linker, acid sensitive hydrazone linker, a linker comprising disulfides, a thioether liner, a glutathione-sensitive disulfide linker, a linker containing a sugar molecule or moiety, a glucuronide containing linker, a glycosidase cleavable linker, a phosphatase cleavable linker, an esterase cleavable linker, a hydrolysis cleavable linker, a monodispersed PEG linker, a PEO linker, a poly-PEG linker, dipeptides such as valine-citrulline, valine-alanine, polypeptides such as glycine-glycine-phenylalanine-glycine, Self-immolative linkers, such as p-aminobenzyloxycarbonyl (PABC), or a combination thereof. The term “cleavable bond” can comprise an enzymatically-cleavable peptide bond, acid sensitive hydrazone bond, disulfide bond, thioether bond, glutathione-sensitive disulfide bond, sugar molecule or moiety, glucuronide bond, glycosidase cleavable bond, phosphatase cleavable bond, esterase cleavable bond, hydrolysis cleavable bond, monodispersed PEG (polyethyleneglycol) (PEG linker) or PEO (polyethyleneoxide) (PEO linker), poly-PEG bond, dipeptides bond, polypeptides bonds such as glycine-glycine-phenylalanine-glycine, p-aminobenzyloxycarbonyl (PABC) bond, or a combination thereof. In some cases, the payload bioactive agent (PBA) can be covalently linked to the targeting bioactive agent (TBA) indirectly via a non-cleavable linker. Examples of non-cleavable linkers commonly used in ADC can include maleimidocaproyl or SMCC through thioether bond and amide bond.

In some cases, the pharmaceutical composition can further comprise a linker L1, a linker L2, or a combination thereof, wherein the linker L1 is covalently linked to the TBA and covalently linked to the reacted first terminal group of the polymer, the linker L2 is covalently linked to the PBA and covalently linked to the reacted second terminal group of the polymer, and at least one of the linker L1 and the linker L2 comprises a cleavable linker that is cleavable in vivo or in the target cell.

In some cases, the cleavable linker can comprise an enzymatically-cleavable peptide linker, acid sensitive hydrazone linker, a linker comprising disulfides, a disulfide linker, a thioether liner, a glutathione-sensitive disulfide linker, a linker containing a sugar molecule or moiety, a glucuronide containing linker, a glycosidase cleavable linker, a phosphatase cleavable linker, an esterase cleavable linker, a hydrolysis cleavable linker, a monodispersed PEG linker, a PEO linker, a poly-PEG linker, a GGFG linker, a VAPAB linker, a dipeptide, a valine-citrulline linker, a valine-alanine linker, a polypeptide linker, p-aminobenzyloxycarbonyl (PABC) linker, or a combination thereof.

In some cases, each of the linker L1, the linker L2, or a combination thereof, can comprise the cleavable linker disclosed herein.

In some cases, the cleavable linker can consist of an enzymatically-cleavable peptide linker, acid sensitive hydrazone linker, a linker comprising disulfides, a disulfide linker, a thioether liner, a glutathione-sensitive disulfide linker, a linker containing a sugar molecule or moiety, a glucuronide containing linker, a glycosidase cleavable linker, a phosphatase cleavable linker, an esterase cleavable linker, a hydrolysis cleavable linker, a monodispersed PEG linker, a PEO linker, a poly-PEG linker, a GGFG linker, a VAPAB linker, a dipeptide, a valine-citrulline linker, a valine-alanine linker, a polypeptide linker, p-aminobenzyloxycarbonyl (PABC) linker, and a combination thereof.

In some cases, the pharmaceutical composition can comprise an enzymatically-cleavable peptide linker that comprises the GGFG linker, the VAPAB linker, the dipeptide, the valine-citrulline linker, the valine-alanine linker, the polypeptide, p-aminobenzyloxycarbonyl (PABC) linker, or a combination thereof.

In some cases, each of the linker L1, the linker L2, or a combination thereof, can comprise the enzymatically-cleavable peptide linker comprising the GGFG linker, the VAPAB linker, the dipeptide, the valine-citrulline linker, the valine-alanine linker, the polypeptide, p-aminobenzyloxycarbonyl (PABC) linker, or a combination thereof.

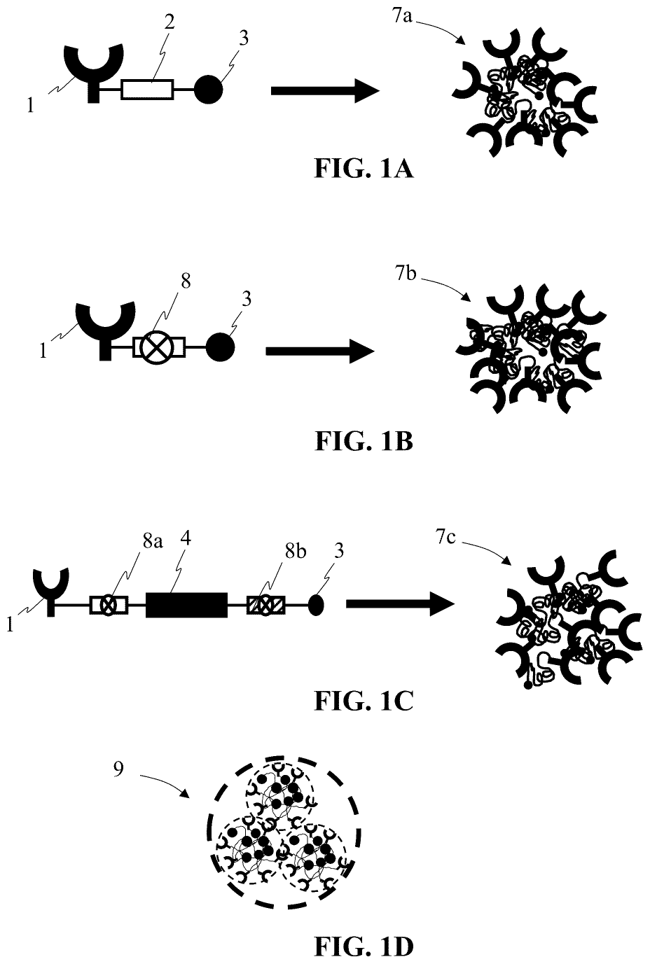

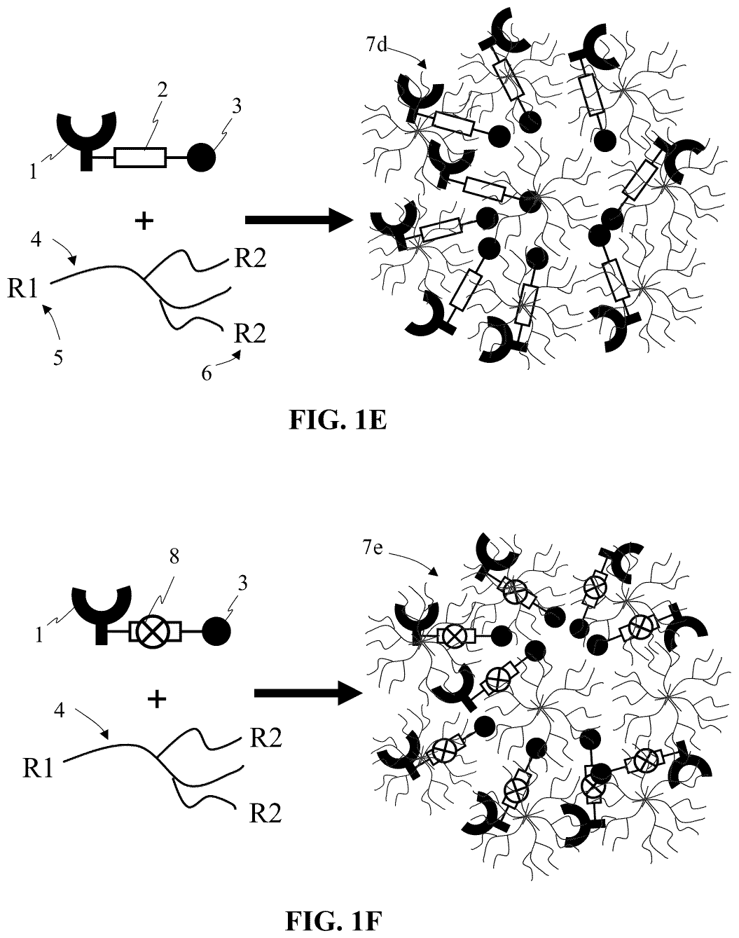

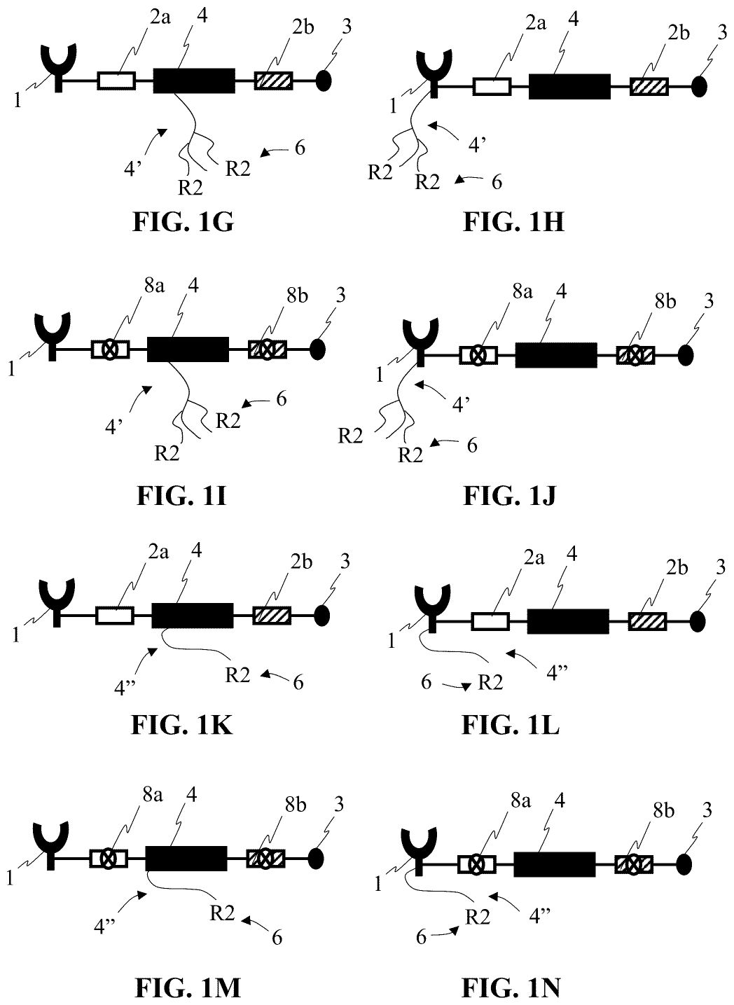

Some representative examples of the pharmaceutical compositions are shown in FIG. 1A-FIG. 1N. In some cases, the pharmaceutical composition can comprise nanoaggregates having particle sizes in a range of from 1 nm to 140 nm. The pharmaceutical composition can comprise a targeting bioactive agent (TBA) (1); a linker (2), and a payload bioactive agent (PBA) (3). The TBA (1), linker (2), and PBA (3) can form nanoaggregates (7a) upon mixing (FIG. 1A). In some cases, the pharmaceutical composition can comprise a cleavable linker (8) and form cleavable nanoaggregates (7b) (FIG. 1B). In some cases, the pharmaceutical composition can further comprise a polymer (4) covalently linked to two cleavable linkers (8a and 8b) forming nanoaggregates (7c) having the TBA (1), cleavable linkers (8a and/or 8b), polymer (4), and PBA (3) (FIG. 1C). The nanoaggregates can further form nanoaggregates complex (9) that can comprise 2 or more aggregates (FIG. 1D). The polymer can comprise one or more first terminal groups (R1) (5) and one or more second terminal groups (R2) (6). In some cases, the polymer can comprise one first terminal group (R1) and two or more second terminal groups (R2). In some cases, the pharmaceutical composition can comprise a polymer (4) non-covalently associated with a TBA (1), a linker (2) and a PBA (3) forming nanoaggregates (7d) (FIG. 1E). In some cases, the pharmaceutical composition can comprise a polymer (4) non-covalently associated with a TBA (1), a cleavable linker (8) and a PBA (3) forming nanoaggregates (7e) (FIG. 1F). R1 and R2 represent the first terminal group and the second terminal group, respectively. Polymer disclosed herein can form nanoaggregates in aqueous solutions as described at least in U.S. Pat. No. 10,688,048. In some cases, the targeting bioactive agent can be an antibody, such as a monoclonal antibody (mAb) and the payload bioactive agent can be a drug forming and an antibody-drug conjugate (ADC).

In some cases, pharmaceutical composition of this disclosure can further comprise a polymer side chain that can be covalently linked to the TBA, the polymer, or a combination thereof, wherein the polymer side chain can comprise polyoxazoline (POX) comprising a linear portion, a branched portion, or a combination thereof, and wherein the polyoxazoline (POX) comprises poly(2-methyloxazoline), poly(2-ethyloxazoline), poly(2-propyloxazoline), poly(isopropyloxazoline), or a combination thereof.

In some cases, an ADC can comprise a polymer side chain attached to the polymer that links the antibody and the PBA and the polymer side chain is free from any PBAs. In some cases, an ADC can comprise a polymer side chain attached to the antibody and the polymer side chain is free from any PBAs. In some cases, an ADC can comprise a cleavable linker and a polymer side chain attached to the polymer that links the antibody and the PBA and the polymer side chain is free from any PBAs. In some cases, an ADC can comprise a cleavable linker and a polymer side chain attached to the antibody and the polymer side chain is free from any PBAs.

In some cases, one or more polymer side chains (4′) can each be covalently linked to the polymer as schematically shown in FIG. 1G and FIG. 1I. In some cases, one or more polymer side chains (4′) can be covalently linked to the TBA, such as an antibody, as schematically shown in FIG. 1H and FIG. 1J. In some cases, one or more linear polymer side chains (4″) can each be covalently linked to the polymer as schematically shown in FIG. 1K and FIG. 1M. In some cases, one or more polymer side chains (4″) can be covalently linked to the TBA, such as an antibody, as schematically shown in FIG. 1L and FIG. 1N.

The polymer side chains can be linked to the antibody or the polymer with commonly used methods. In some cases, one or more polymer side chains can be linked to an antibody via —S bonds such as via lysine residues, —N bond such as via cysteine residues, glycol bond, modified glycol bond, GlcNAc (N-acetylglucosamine), substituted GlcNAc, GlcNAc-galactose, GlcNAc-mannose, GlcNAc-glucose, GlcNAc-glucuronic acid, GlcNAc-fucose, GlcNAc-N-acetylneuraminic acid, site specific bond, random bond, bioconjugations to other amino acid residues of the antibody, such as tyrosine (Tyr), tryptophan (Trp), and methionine (Met), conjugations via genetically engineered non-natural amino acids (NNAA), enzymatic conjugations, or a combination thereof, as disclosed herein.

In some cases, the polymer can comprise polyoxazoline (POX) having a branched portion that can comprise two or more branches, and wherein at least one of the two or more branches can be the polymer side chain and can be free from the PBA.

In some cases, the polymer can be covalently linked to the targeting bioactive agent (TBA) and the payload bioactive agent (PBA), wherein the polymer can comprise, prior to reacting to the TBA or the PBA, at least one first terminal group modified with H or a hydrophobic moiety and a second terminal group modified with a hydrophilic moiety, wherein the first terminal group comprises in a range of from 1% to 99% of H and 1% to 99% of the hydrophobic moiety that comprises saturated or unsaturated aliphatic hydrocarbon having 0 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or a combination thereof, and wherein the polymer can comprise a reacted first terminal group and a reacted second terminal group; and wherein the polymer is positioned between the targeting bioactive agent (TBA) and the payload bioactive agent (PBA) with the reacted first terminal group covalently linked to the TBA and the reacted second terminal group covalently linked to the PBA, directly or indirectly. The term “directly” used herein means that two molecules are directly reacted and forming a covalent bond. The term “indirectly” used herein means that two molecules are reacted via an additional one or more molecules and forming a covalent bond with the additional molecule(s) positioned between and linking the two molecules. The term “reacted first terminal group” refers to a functional group or a residue of a functional group that has been reacted with a reactant. For example, a reacted first terminal group can be a —CH— that is a reaction product of a first terminal group —CH₂. In another examples, a reacted first terminal group can be a hydrophobic moiety that comprises saturated or unsaturated aliphatic hydrocarbon having 0 to about 22 carbons, an aromatic hydrocarbon, or a combination thereof, with at least one of the active H reacted with a reactant. The term “reacted second terminal group” refers to functional group or a residue of a functional group that has been reacted with a reactant. For example, a reacted second terminal group can be —NH— that is a reaction product of a —NH₂ group, a —O— that is a reaction product of an —OH group, a —COO— that is a reaction product of a —COOH group, and so on. In some cases, the reacted second terminal group can be a reaction product of the group selected from an amine, amide, imine, imide, carboxyl, hydroxyl, ester, ether, acetate, phosphate, ketone, aldehyde, sulfonate, or a combination thereof; and a combination thereof. In some cases, the first terminal group comprises a hydrophobic moiety that comprises 0 carbon, i.e., the first terminal group is free from the hydrophobic moiety.

In some cases, the polymer can be non-covalently associated with the targeting bioactive agent (TBA) and the payload bioactive agent (PBA), wherein the polymer can comprise at least one first terminal group modified with H or a hydrophobic moiety and a second terminal group modified with a hydrophilic moiety, wherein the first terminal group comprises in a range of from 1% to 99% of H and 1% to 99% of the hydrophobic moiety that comprises saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or a combination thereof.

In some cases, the pharmaceutical composition can comprise a polymer that comprises polyoxazoline (POX) that is non-covalently associated with the targeting bioactive agent (TBA) and the payload bioactive agent (PBA) forming nanoaggregates, wherein the polyoxazoline (POX) comprises a linear portion, a branched portion, or a combination thereof, and wherein the polyoxazoline (POX) comprises poly(2-methyloxazoline), poly(2-ethyloxazoline), poly(2-propyloxazoline), poly(isopropyloxazoline), or a combination thereof, and wherein the polyoxazoline (POX) comprises at least one first terminal group modified with H or a hydrophobic moiety and at least one second terminal group modified with a hydrophilic moiety, wherein the first terminal group comprises in a range of from 1% to 99% of H and 1% to 99% of the hydrophobic moiety that comprises saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof; or a combination thereof.

In some cases, in a range of from 1% to 100% of the second terminal group can be free from primary amine.

In some cases, in a range of from 1% to 100% of the second terminal group can comprise hydroxyl group.

In some cases, the targeting bioactive agent (TBA), payload bioactive agent (PBA), and the polymer are aggregated to form nanoaggregates having sizes in a range of from 1 nm to 140 nm.

In some cases, the pharmaceutical composition can comprise nanoaggregate having a size less than 120 nm before lyophilization. The nanoaggregate can comprise the aforementioned targeting bioactive agent (TBA), the payload bioactive agent (PBA) covalently linked to the targeting bioactive agent (TBA) directly or indirectly, and the polymer.

In some cases, the pharmaceutical composition can comprise nanoaggregate having a size less than 140 nm before lyophilization. In particular cases, the pharmaceutical composition can comprise nanoaggregate having in a range of from 1 to 140 nm, 1 to 120 nm, 1 to 100 nm, 1 to 80 nm, 1 to 60 nm, 1 to 40 nm, 1 to 20 nm, 1 to 15 nm, 1 to 10 nm, 1 to 8 nm, 1 to 6 nm, 2 to 120 nm, 4 to 120 nm, 4 to 100 nm, 4 to 80 nm, 4 to 60 nm, 4 to 40 nm, 4 to 30 nm, 4 to 20 nm, 4 to 15 nm, before lyophilization. In some cases, the pharmaceutical composition can comprise nanoaggregate having in a range of from 15 to 120 nm, 15 to 100 nm, 15 to 80 nm, 15 to 60 nm, 15 to 40 nm, 15 to 30 nm, 15 to 20 nm, 10 to 120 nm, 10 to 100 nm, 10 to 80 nm, 10 to 60 nm, 10 to 40 nm, 10 to 30 nm, 10 to 20 nm, 5 to 120 nm, 5 to 100 nm, 5 to 80 nm, 5 to 60 nm, 5 to 40 nm, 5 to 30 nm, 5 to 20 nm, 5 to 10 nm, 2 to 120 nm, 2 to 100 nm, 2 to 80 nm, 2 to 60 nm, 2 to 40 nm, 2 to 30 nm, 2 to 20 nm, 2 to 15 nm, 2 to 10 nm, and 2 to 5 nm, before lyophilization. In some cases, the pharmaceutical composition can comprise nanoaggregates having a size less than 120 nm before lyophilization, wherein the nanoaggregates comprise the bioactive agent (TBA), the payload bioactive agent (PBA), and the polymer. The size of nanoaggregate can be measured using a dynamic light scattering (DLS) method. Other methods known to those skilled in the art can also be used.

In some cases, the pharmaceutical composition can comprise a tumor cytotoxicity to tumor cells and a non-tumor cytotoxicity to non-tumor cells, wherein the tumor cytotoxicity comprises Ag⁺ tumor cytotoxicity to tumor cells having the tumor antigen (Ag⁺ tumor cells) and Ag⁻ tumor cytotoxicity to tumor cells free from the tumor antigen (Ag⁻ tumor cells).

In some cases, the pharmaceutical composition can comprise the Ag⁻ tumor cytotoxicity to the Ag⁻ tumor cells in the absence of the Ag⁺ tumor cells.

In some cases, the Ag⁻ tumor cytotoxicity can be at least 20% higher than the non-tumor cytotoxicity.

In some cases, the Ag⁻ tumor cytotoxicity, Ag⁺ tumor cytotoxicity, and non-tumor cytotoxicity, each can be measured in a cell-based assay.

In some cases, the Ag⁻ tumor cytotoxicity can be at least 20% higher than the non-tumor cytotoxicity, i.e., at least 20% more Ag⁻ tumor cells can be killed than that of non-tumor cells. In another word, cell survival of the non-tumor cells can be at least 20% more than that of the Ag⁻ tumor cells. In some cases, Ag⁻ tumor cytotoxicity can be at least 20%, 30%, 40%, 50%, 60%, 70%, 80%, 90% or 100% higher than the non-tumor cytotoxicity.

Each of the ranges is intended as a continuous range including every value between the minimum and maximum values and including the minimum and maximum cited values.

The pharmaceutical composition can comprise the Ag⁻ tumor cytotoxicity to Ag⁻ tumor cells in the absence of any Ag⁺ tumor cells. In another word, the Ag⁻ tumor cytotoxicity of the Nano-ADC of this disclosure to Ag⁻ tumor cells can be independent of any Ag⁺ tumor cells.

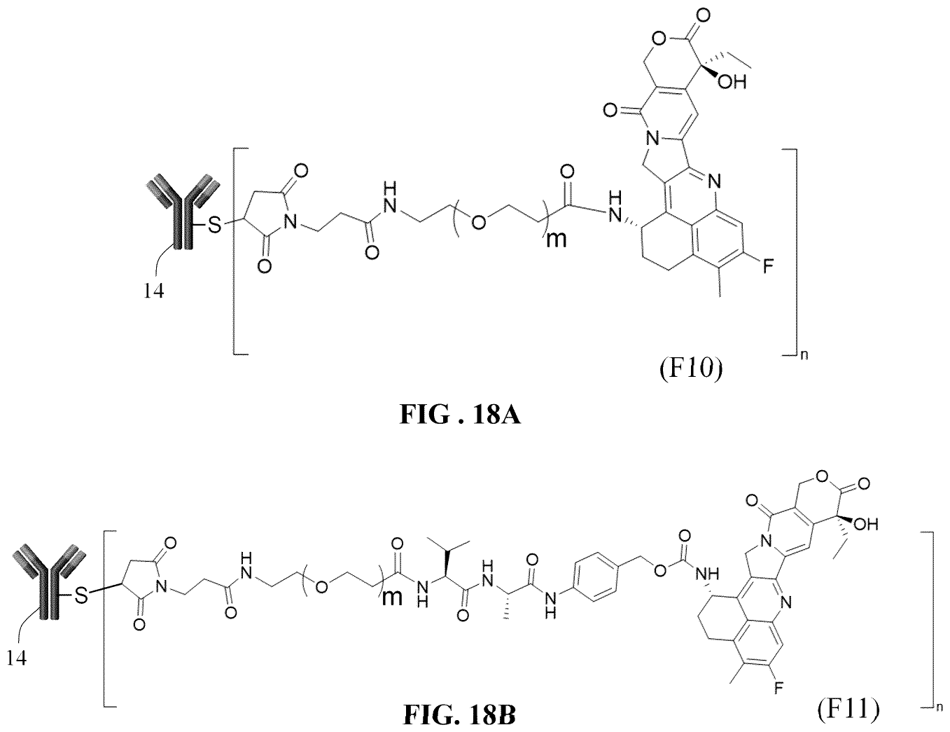

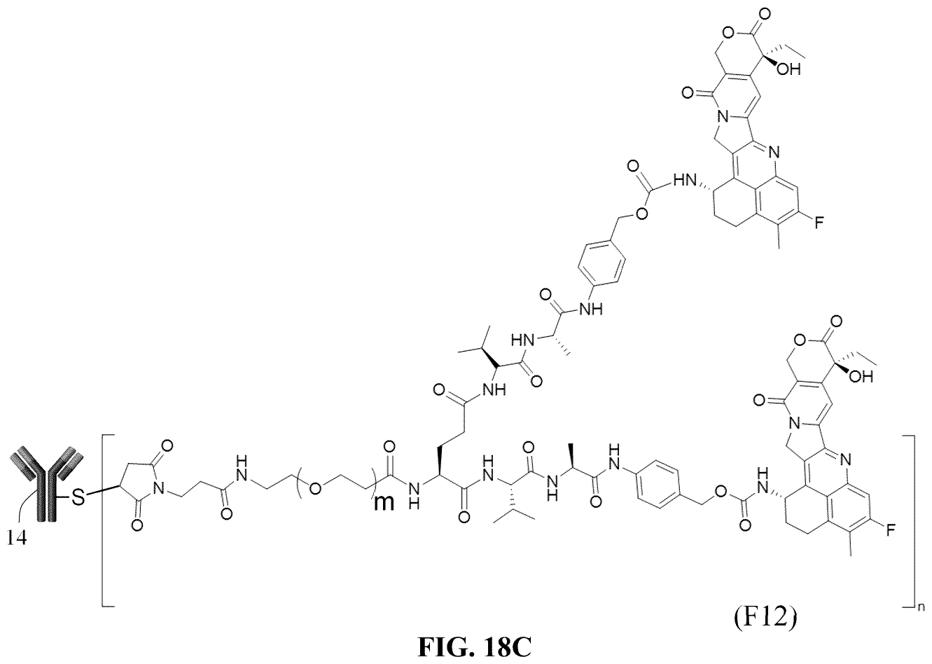

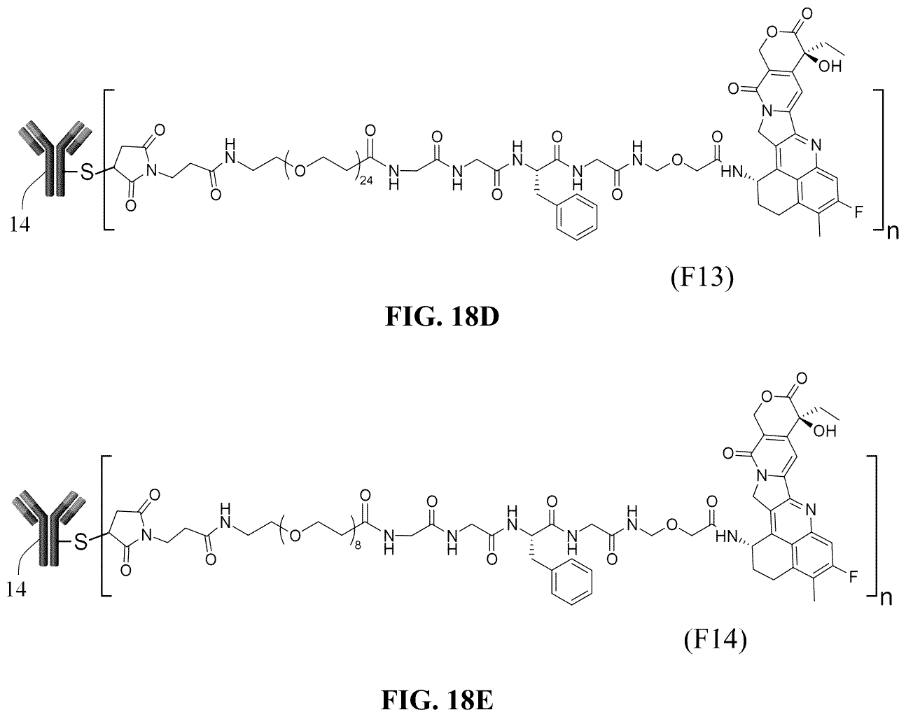

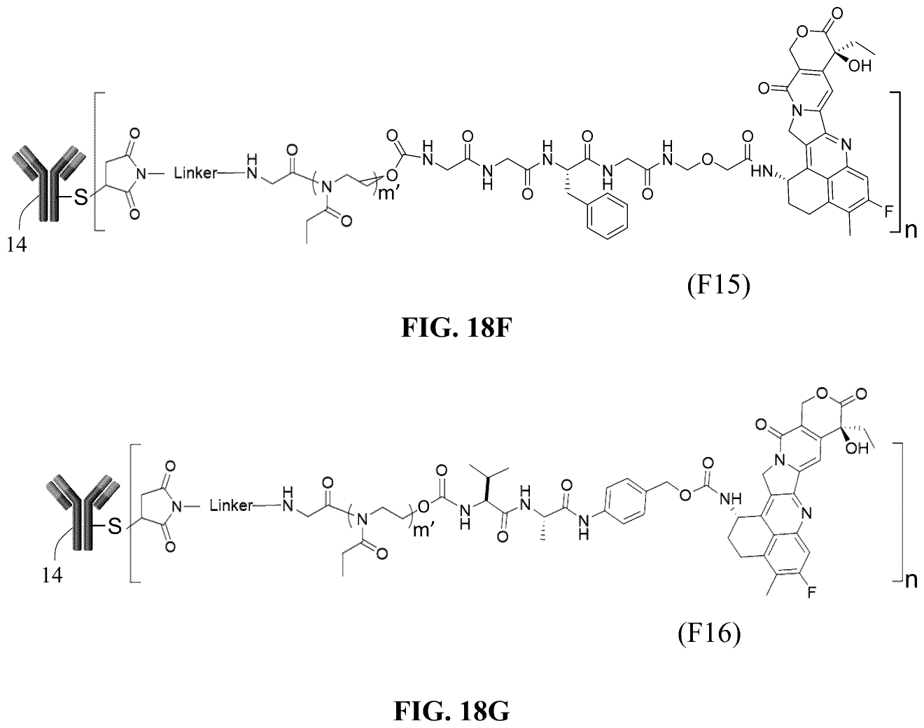

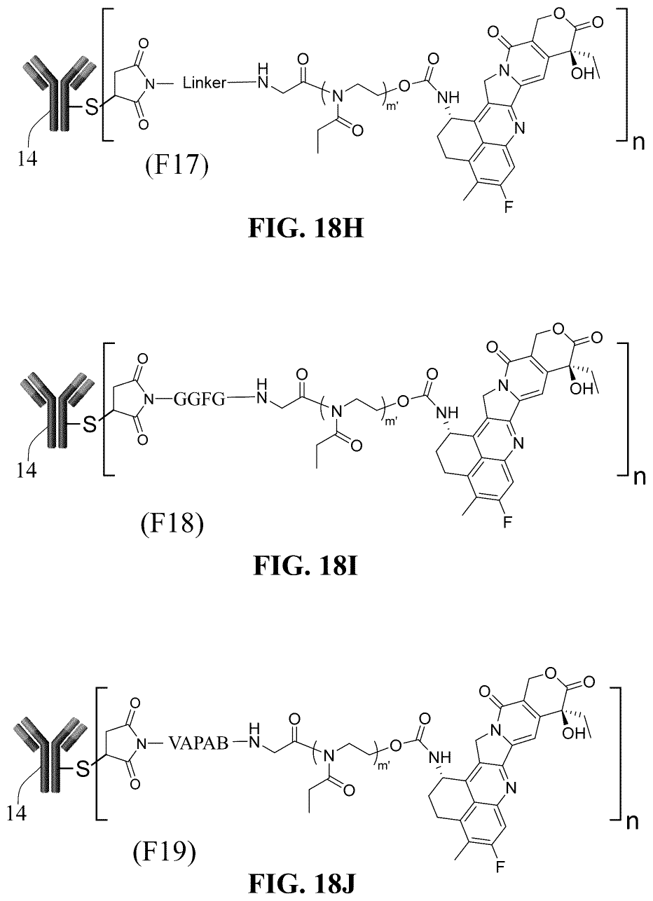

For a traditional ADC, the drug, i.e., the payload bioactive agent (PBA), from an ADC can be released either from the target cell following internalization and degradation of the ADC or in the extracellular space. The drug can then be taken up by and kills surrounding or bystander cells resulting in “bystander cytotoxicity”. These surrounding or bystander cells themselves may or may not express the ADC target antigen, i.e., can be Ag⁻ tumor cells or Ag⁻ normal cells. Traditional ADCs, such as the aforementioned ENHERTU® ADC, exhibit no Ag⁻ tumor cytotoxicity to Ag⁻ tumor cells in the absence of any Ag⁺ tumor cells. Refer to FIG. 18A and EXAMPLES for details.

The Ag⁻ tumor cytotoxicity, the Ag⁺ tumor cytotoxicity, and the non-tumor cytotoxicity, each can be measured in a cell-based assay. In one example, a cell viability assay can be used. In another example, a cell death assay can be used. Commercial assay kits, such as those available from Thermo Fisher Scientific can be suitable.

In any of suitable cell-based assays, only Ag⁻ tumor cells can be used in the absence of any Ag⁺ tumor cells. Traditional ADCs, such as Enhertu ADC having a monoclonal antibody trastuzumab and a cytotoxic drug deruxtecan, exhibit no such Ag⁻ tumor cytotoxicity in the absence of any Ag⁺ tumor cells.

Multiple methods can be employed to calculate the differences in cytotoxicity. One way to calculate a difference (in percentage number) of the Ag⁻ tumor cytotoxicity (Viability %: V_{AgNT}) v.s. the non-tumor cytotoxicity (Viability %: Vₙₒᵣₘₐₗ) can be based on cell viability using the following formula:

difference=V_{AgNT.}−Vₙₒᵣₘₐₗ.

In some cases, the Nano-ADC of this disclosure can exhibit Ag⁻ tumor cytotoxicity at least 20%, 30%, 40%, 50% higher than that of the non-tumor cytotoxicity, i.e., V_{AgNT.} is at least 20%, 30%, 40%, 50% lower than Vₙₒᵣₘₐₗ if a cell viability is used. On the other word, the Nano-ADC of this disclosure can exhibit Ag⁻ tumor cytotoxicity with a cell death rate at least 20%, 30%, 40%, 50% higher than that of the non-tumor cytotoxicity, if a cell death rate is used.

In some cases, since each of the traditional ADCs is configured to have a mAb targeting a tumor antigen (Ag) on tumor cells, a cell-based assay using Ag⁻ tumor cells can be used to measure the Ag⁻ tumor cytotoxicity. Not wishing to be bound by any particular calculation methods, in some cases, Applicants use cell viability to calculate cytotoxicity. For example, specific Ag⁻ tumor cells, such as MDA-MB231 cells of a triple-negative breast cancer (TNBC) (HER2⁻) cell line can be used to assay the Ag⁻ tumor cytotoxicity of the ADC comprising an anti-HER2 antibody. In such assays, a traditional ADC, such as ENHERTU® can be used as a control to compare with the cytotoxicity of the Nano-ADC of this disclosure and the cytotoxicity of ENHERTU® towards HER2⁻ cells, such as the MDA-MB231 cells or other non-HER2 overexpressing cells (See EXAMPLE 1).

In some cases, the pharmaceutical composition can comprise the TBA, wherein the TBA can comprise a binding affinity to a target molecule selected from a gene or a gene product of PD-1, PD-L1, 4-1BB, 5T4 (5T4 oncotrophoblast glycoprotein), ABL1, ABL2, ACVR1, AKR1C3, AKT, ALK, ASCL1, ASNS, Asparagine, ASGR1, ASGPR1, ATM, ATR, ATRX, AURKA, AURKB, AXL, B7H3 (CD276 antigen), B7H4 (V-set domain-containing T-cell activation inhibitor 1), B7H7 (HHLA2), BAK1, BAX, BCL2 family members (BCL2, BCL2L1, MCL1, BCL2A1, BAK1, BAX), BCL2A1 (BFL1), BCMA (TNFRSF17), BCOR, BET bromodomain family, BIRC5 (survivin), BMPR, BRAF, BRD1, BRD4, CCND1, CCND2, CCR8, CD19, CD22, CD25, CD274 (PD-L1), CD276 (B7-H3), CD3, CD16a, CD28, CD33, CD37, CD38, CD40, CD40L, CD47, CD52, CD7, CD70, CD73, CD79B, CD200R1 (Cell surface glycoprotein OX2 receptor 1), CDC7, CDK12, CDK2, CDK4, CDK6, CDK7, CDK9, CHEK1 (CHK1), CK1 (casein Kinase 1), CK2 (casein kinase 2), CLDN18.2, CRBN [cereblon E3 ubiquitin ligase], CREBBP/EP300, CRTAM, CSF1R, CSF2 (GM-CSF), CTAGlB (NY-ESO-1), CTLA4, CTNNB1, CXCL10, CXCR4, DDX3X, DLK1, DLL3 (Delta-like Ligand 3), DNA (alkylators), DNA-PK, DNMT (DNA methyl transferase), DOT1L, EED, EGFR, EGFRvIII (EGFR variant III), EpCAM (TROP1), EPHA2, ERBB2 (HER2), ETS gene fusions, EWSR1-FLI1, EZH2, F3 (tissue factor), FGFR, FLT3, FOLR1 (folate receptor alpha), Gamma secretase, GD2 (disialoganglioside), GFI1, GFIlB, GPC2, GPC3, GPNMB, GSK3, HAVCR2 (TIM3), TIMD4 (TIM4), HDAC, HIF1A, Hippo pathway (YAP1, WWTR1 (TAZ), TEADs), Histone H3, HSP90, IDH1, IDH2, IDO1, IFNG (interferon gamma), IGF1R, IL13RA2, IL2, IL3RA (CD123), IL6, IL23p19, Inhibitor of apoptosis (IAP) proteins, JAK1, JAK2, JAK3, KAT6A (MYST3), KDM1A (LSD1), KDM4A, KIT, KMT2A (MLL), KMT2E (MLL5), LAG3, LIFR, LIN28B, LRRC15, MAGEA3, MAP2K1 (MEK1), MAP2K2 (MEK2), MAPK3 (ERK1), MAPK1 (ERK2), MCL1, MDM2, MEN1 (menin), MET, MGMT, MS4A1 (CD20), MSLN (mesothelin), MTOR, mTORC1, mTORC2, MYC, MYCN, NAMPT, NCAM1 (CD56), MUC1, MUC16, NEDD8 activating enzyme (NAE), Neoantigens, NF-kappa-B, NKp30, NKp46, NOTCHI, NR5A1 (steroidogenic factor 1), NT5C2, NTRK, NUTM1 gene fusions, ODC1, OLIG2, PARP, PAX5, PAX-FOXO1, PDCD1 (PD-1), PD-L1, PDGFRA, PDGFRB, PDPK1 (3-phosphoinositide dependent protein kinase 1), PIK3CA (PI3K-alpha), PIK3CD (PI3K-delta), PIM1, PKA (protein kinase A), PKC (protein kinase C), PLK1, PML-RARA, PPM1D (WIP1), PRAME, PRDM1, PRDM10, PRDM8, PRMT2, PRMT5, Proteasome, PSMA, PTEN, PTK2 (FAK), PTPN (protein tyrosine phosphatase), PTPN11 (SHP2), RAS, RELA, RET, RIGI (DDX58), RNA polymerase (RNApol) I, ROR1, ROR2, ROS1, RPA3, SARS-Cov-2, SH2B3, SLC16A1 (MCT1), SMO, SMYD3, SSTR (somatostatin receptor), STAT2, STAT3, STEAP1, STING1 (STING), SUZ12, SWI/SNF, SYK, SYT-SSX, TERT, TET2, TGF-beta, TNFRSF4 (OX40), OX40L, TNFRSF8 (CD30), TNFSF10 (TRAIL), TOP1, TOP2 (DNA topoisomerase I/II), TROP2, TP53, TSLP, Tubulin, TYK2, TYMS, VEGF, VEGFR, WDR5, WEE1, WT1, XPO1 (Exportin 1), YAP1, ZBTB17 (MIZ-1), or a combination thereof. The TBA can comprise a binding affinity to a target molecule listed above in vivo, such as in cells or in a subject or in vitro, such as in cultures or in cell free assays.

In some cases, the pharmaceutical composition can comprise the TBA, wherein the TBA can be selected from a peptide, a protein, DNA, RNA, nucleic acids, oligo nucleic acids, mRNA, siRNA, sgRNA, a monoclonal antibody, a fragment of a monoclonal antibody, a polyclonal antibody, a fragment of a polyclonal antibody, a synthetic antibody, a fragment of a synthetic antibody, antigen-binding portions thereof, an agonist, an activator, an inhibitor, an antagonist, a ligand of cell surface receptor, a naked nucleic acid, a nucleic acid, a ribozyme, a virus, a virus-like particles and a combination thereof, Alemtuzumab, alemtuzumab, an antisense nucleic acid, anti-CD19 antibodies, anti-CD20 antibodies, anti-CTLA-4 (cytotoxic T lymphocyte-associated antigen) antibodies, anti-LAG3 (lymphocyte activation gene-3) antibodies, anti-PD1 antibodies KEYTRUDA® (pembrolizumab), anti-PD-L1 antibodies, anti-TIM-3 (T cell immunoglobulin and mucin domain-3) antibodies, Atezolizumab, BAVENCIO® (avelumab), Bevacizumab, bevacizumab, Blinatumomab, Brentuximab, Cetuximab, cetuximab, chimeric antigen receptor or chimeric antigen T-cell receptor (CAR-T), cytokines, Daratumumab, Denosumab, Dinutuximab, Durvalumab, Elotuzumab, gemtuzumab, granulocyte colony stimulating factor (G-CSF), Ibritumomab, IMFINZI® (durvalumab), Inotuzumab, interferon α, interferon α2a, interleukins, Ipilimumab, lectins, Necitumumab, Neupogen (Filgrastim), Obinutuzumab, Ofatumumab, Olaratumab, OPDIVO® (nivolumab), Panitumumab, Pembrolizumab, Pertuzumab, Ramucirumab, Rituximab, rituximab, Siltuximab, Tislelizumab (BGB-A317), Toripalimab, siRNAs, T-cell receptor (TCR), TECENTRIQ® (atezolizumab), tositumomab, trastuzumab, anti-EGFR, anti-PSMA, a member of a small molecule binding pair, and a combination thereof.

In some cases, the TBA can be selected from Alemtuzumab, Atezolizumab, Avelumab, Bevacizumab, Blinatumomab, Brentuximab, Cetuximab, Daratumumab, Denosumab, Dinutuximab, Durvalumab, Elotuzumab, Gemtuzumab, Ibritumomab, Inotuzumab, Ipilimumab, Necitumumab, Nivolumab, Obinutuzumab, Ofatumumab, Olaratumab, Panitumumab, Pembrolizumab, Pertuzumab, Ramucirumab, Rituximab, Siltuximab, Tislelizumab (BGB-A317), Toripalimab, Trastuzumab, and a combination thereof.

In some cases, the TBA can be selected from Pertuzumab, Trastuzumab, and a combination thereof.

In some cases, the pharmaceutical composition can comprise the Ag⁺ tumor cytotoxicity to HER2⁺ tumor cells and the Ag⁻ tumor cytotoxicity to HER2− tumor cells, wherein the Ag⁻ tumor cytotoxicity is measured in a cell-based assay with the HER2⁻ tumor cells in the absence of the HER2⁺ tumor cells.

In some cases, the PBA can comprise a natural or synthetic small molecule-based drug, inorganic-based drug, a toxin molecule having cytotoxicity, a molecule having cytotoxicity to the target cell, a derivative thereof, or a combination thereof.

In some cases, the PBA can comprise 5-Fluorouracil (5-FU), alkylators, anti-metabolites, cabazitaxel, Calicheamicin, Calicheamicin derivatives, camptothecin, camptothecin derivatives, capecitabine, cell division inhibitors or microtubule inhibitors, ciprofloxaxin, cyclophosphamide, deruxtecan, deruxtecan derivatives, DNA cleavage agent, DNA crosslinking agent, DNA replicating inhibitor, docetaxel, dolastatin analogs, doxorubicin, duocarmycin analogs, etoposide, everolimus, exatecan, exatecan derivatives, topoisomerase inhibitors, gemcitabine, irinotecan (CPT-11), larotaxel, maytansinoids (DM1, DM4), milataxel, MMAE (monomethyl auristatin E), MMAF (monomethyl auristatin F), MMAH (monomethyl auristatin H), mTOR inhibitor, one or more ATP-competitive mTORC1/2 inhibitors, one or more dual PI3K-mTOR inhibitors, ortataxel, ozogamicin, paclitaxel, pemetrexed, pyrrolobenzodiazepine dimer (PBD), rapamycin, ridaforolimus, SN-38, taxane, temsirolimus, tesetaxel, Topoisomerase 1 (Top 1) inhibitor, Topoisomerase 2 (Top 2) inhibitor, topotecan, torin-1, torin-2, vinblastine, vinca alkaloids, vincristine, vinorelbine, vistusertib, zotarolimus, biological toxins, ricin, or a combination thereof.

In some cases, the pharmaceutical composition is a drug for treating or preventing a disease selected from one or more immune disorders, infectious diseases, cancers, and a combination thereof.

In some cases, the pharmaceutical composition can comprise a molar ratio of the TBA to the PBA in a range of from about 1:0.01 to about 1:100.

In some cases, the pharmaceutical composition can comprise a molar ratio of the TBA to the PBA in a range of from about 1:0.01 to about 1:100, 1:0.1 to about 1:100, 1:1 to about 1:100. In particular cases, the pharmaceutical composition can comprise a molar ratio of the TBA to the PBA in a range of from 1:1 to about 1:100, 1:1 to about 1:80, 1:1 to about 1:70, 1:1 to about 1:60, 1:1 to about 1:50, 1:1 to about 1:40, 1:1 to about 1:30, 1:1 to about 1:20, 1:1 to about 1:10, 1:1 to about 1:5, 1:1 to about 1:4, 1:1 to 1:3, 1:1 to 1:2.

In some cases, the pharmaceutical composition can comprise a molar ratio of the TBA to the PBA in a range of 1:2 to 1:100. In particular cases, the pharmaceutical composition can comprise a molar ratio of the TBA to the PBA in a range of from 1:2 to about 1:100, 1:2 to about 1:80, 1:2 to about 1:70, 1:2 to about 1:60, 1:2 to about 1:50, 1:2 to about 1:40, 1:2 to about 1:30, 1:2 to about 1:20, 1:2 to about 1:10, 1:2 to about 1:5, 1:2 to about 1:4, and 1:2 to 1:3.

In some cases, the pharmaceutical composition can comprise a mixture of TBA, wherein some of the TBA can be covalently linked to the PBA and some of the TBA can be non-covalently mixed with the PBA leading to more TBAs than the PBAs. In some cases, some antibody molecules can be free from bound drugs, i.e., free antibody without conjugated to a drug molecule. In some cases, the molar ratio of TBA:PBA can be 100:1, 20:1, 10:1, 5:1, 4;1, 3;1, or 2:1 (on a reverse ratio of 1:0.01, 1:0.1, 1:0.2, 1:0.25, 1:0.3, and 1:0.5). In some cases, the molar ratio of antibody to a drug can be 100:1, 10:1, 5:1, 4;1, 3;1, or 2:1 (on a reverse ratio of 1:0.01, 1:0.1, 1:0.2, 1:0.25, 1:0.3, and 1:0.5).

In some cases, the pharmaceutical composition can comprise one molecule of TBA and at least 2 molecules of the PBA. In some cases, the pharmaceutical composition can comprise one molecule of TBA and at least 3 molecules of the PBA. In some cases, the pharmaceutical composition can comprise one molecule of TBA and at least 4 molecules of the PBA. In some cases, the pharmaceutical composition can comprise one molecule of TBA and at least 5 molecules of the PBA. In some cases, the pharmaceutical composition can comprise one molecule of TBA and at least 6 molecules of the PBA. In some cases, the pharmaceutical composition can comprise one molecule of TBA and at least 8 molecules of the PBA.

In some cases, the nanoaggregate can comprise the bioactive agent (TBA), the payload bioactive agent (PBA) covalently linked to the targeting bioactive agent (TBA) directly or indirectly, and the polymer.

In some cases, the polymer can comprise a polyethylene glycol (PEG), polyoxazoline (POX), or a combination thereof, wherein the polyoxazoline (POX) comprises a linear portion, a branched portion, or a combination thereof, and wherein the polyoxazoline (POX) comprises poly(2-methyloxazoline), poly(2-ethyloxazoline), poly(2-propyloxazoline), poly(2-isopropyloxazoline), poly(2-substituted oxazoline), or a combination thereof. In some cases, the polymer can be selected from a polyethylene glycol (PEG), polyoxazoline (POX), or a combination thereof, wherein the polyoxazoline (POX) comprises a linear portion, a branched portion, or a combination thereof, and wherein the polyoxazoline (POX) comprises poly(2-methyloxazoline), poly(2-ethyloxazoline), poly(2-propyloxazoline), poly(2-isopropyloxazoline), poly(2-substituted oxazoline), and a combination thereof.

In some cases, the polymer can comprise polyethylene glycol (PEG) having monomer units in a range of from 2 to 100, i.e., m=1-100 in formulas disclosed herein. In some cases, the polymer can comprise polyethylene glycol (PEG) having monomer units in a range of from 2 to 36, 2 to 30, 2 to 24, 2 to 20, 2 to 18, 2 to 16, 2 to 14, 2 to 12, 2 to 10, 2 to 8, 2 to 6 and 2 to 4. In some cases, the polymer can comprise polyethylene glycol (PEG) having monomer units in a range of from 12 to 36, 12 to 30, 12 to 24, 12 to 20, 12 to 18, 12 to 16, and 12 to 14. In some cases, the polymer can comprise polyethylene glycol (PEG) having monomer units in a range of from 20 to 36, 20 to 30, 20 to 26 and 20 to 24. In some cases, the polymer can comprise polyethylene glycol (PEG) having 6 monomer units. In some cases, the polymer can comprise polyethylene glycol (PEG) having 8 monomer units. In some cases, the polymer can comprise polyethylene glycol (PEG) having 10 monomer units. In some cases, the polymer can comprise polyethylene glycol (PEG) having 12 monomer units. In some cases, the polymer can comprise polyethylene glycol (PEG) having 14 monomer units. In some cases, the polymer can comprise polyethylene glycol (PEG) having 16 monomer units. In some cases, the polymer can comprise polyethylene glycol (PEG) having 18 monomer units. In some cases, the polymer can comprise polyethylene glycol (PEG) having 20 monomer units. In some cases, the polymer can comprise polyethylene glycol (PEG) having 24 monomer units.

In some cases, the pharmaceutical composition can comprise at least two PBAs that can be the same or different. In some cases, a pharmaceutical composition can comprise an ADC having at least two different PBAs covalently linked to each polymer, such as each PEOX polymer or each PEG polymer, wherein each of the at least two different PBAs can be selected from at least two different natural or synthetic small molecule-based drugs, inorganic-based drugs, toxin molecules having cytotoxicity, molecules having cytotoxicity to target cells, derivatives thereof, or a combination thereof.

In some cases, the pharmaceutical composition can comprise an ADC (i.e., a Nano-ADC disclosed herein) that can comprise at least a first PBA covalently linked to a first polymer and a second PBA covalently linked to a second polymer, wherein the first polymer and the second polymer each is covalently linked to a TBA, wherein the first polymer and the second polymer can be the same or different, wherein the first PBA and the second PAB can be the same or different and each of the at least two different PBAs can be selected from same or different natural or synthetic small molecule-based drugs, inorganic-based drugs, toxin molecules having cytotoxicity, molecules having cytotoxicity to target cells, derivatives thereof, or a combination thereof.

Some examples of the pharmaceutical compositions can be schematically shown in FIG. 1A-FIG. 4D.

In some cases, the pharmaceutical composition can comprise an antibody-drug conjugate (ADC) having a formula selected from Formula F2 to Formula F27:

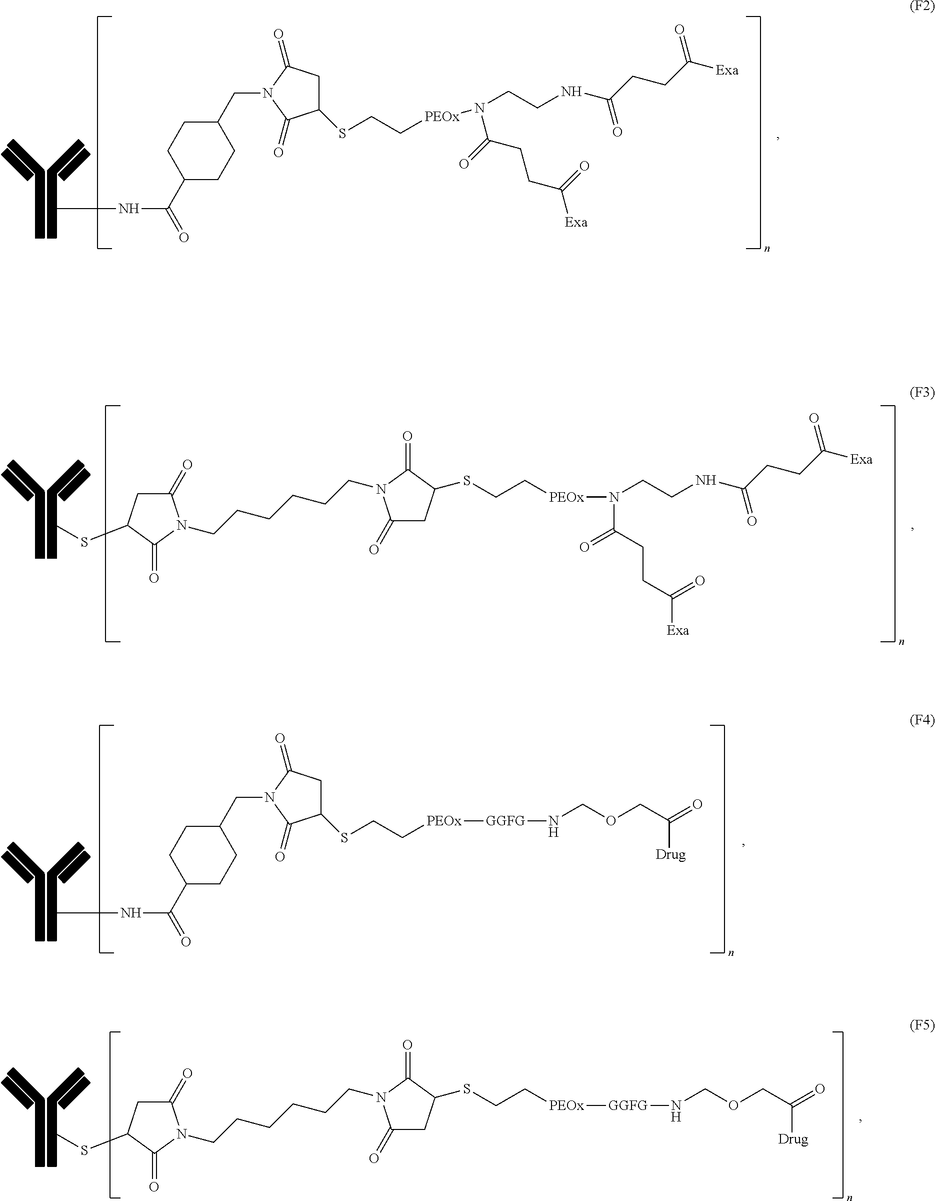

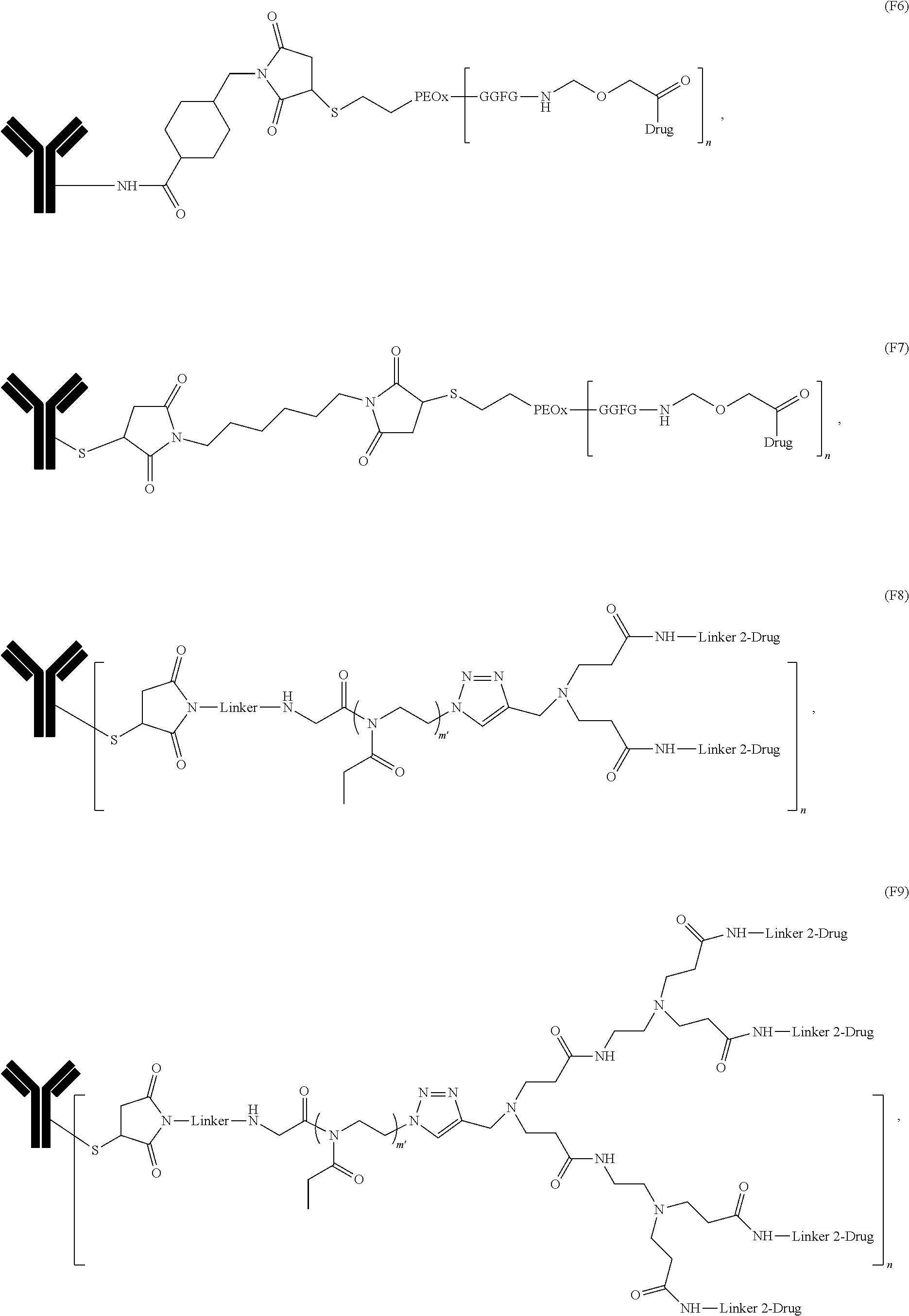

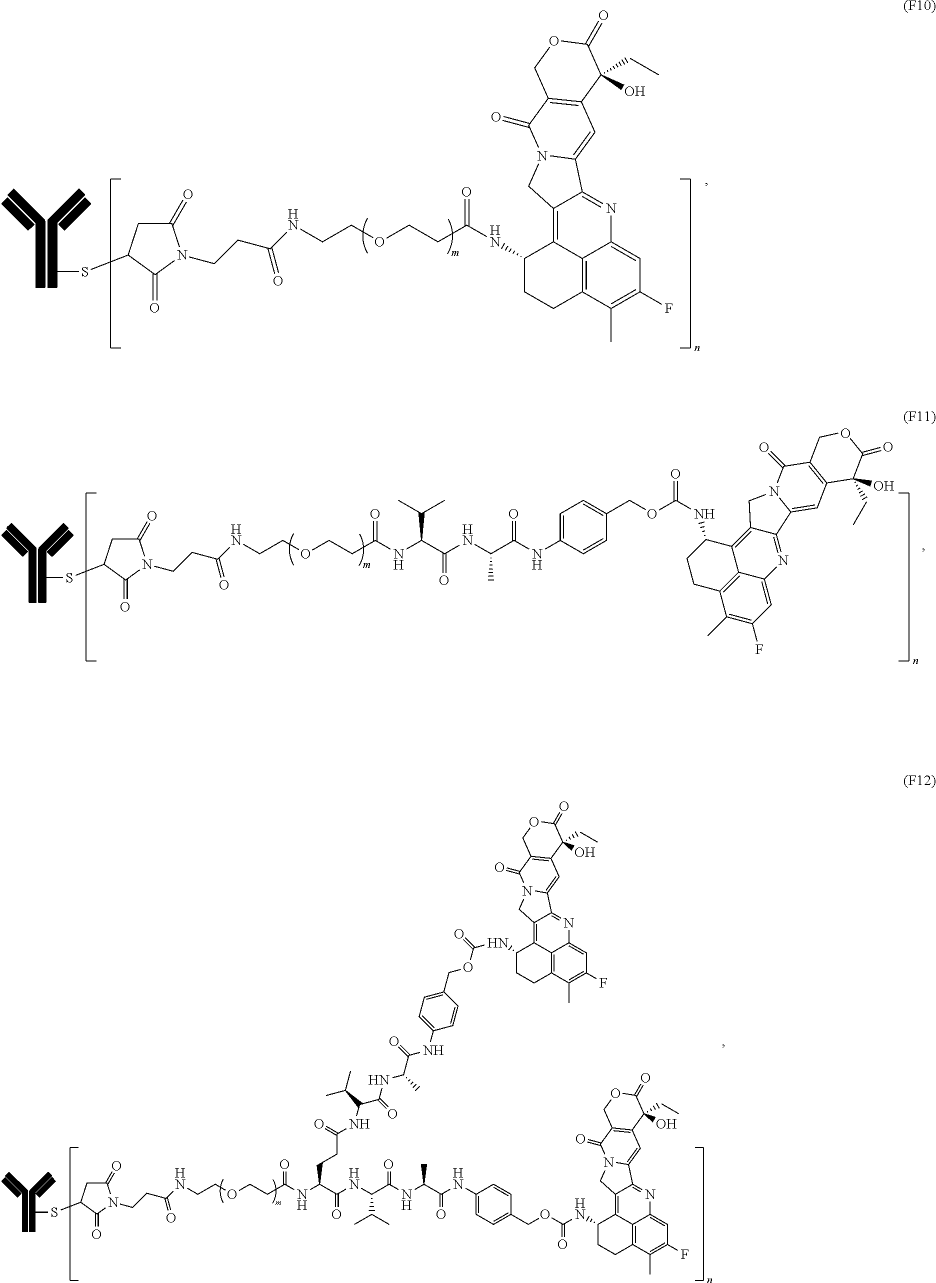

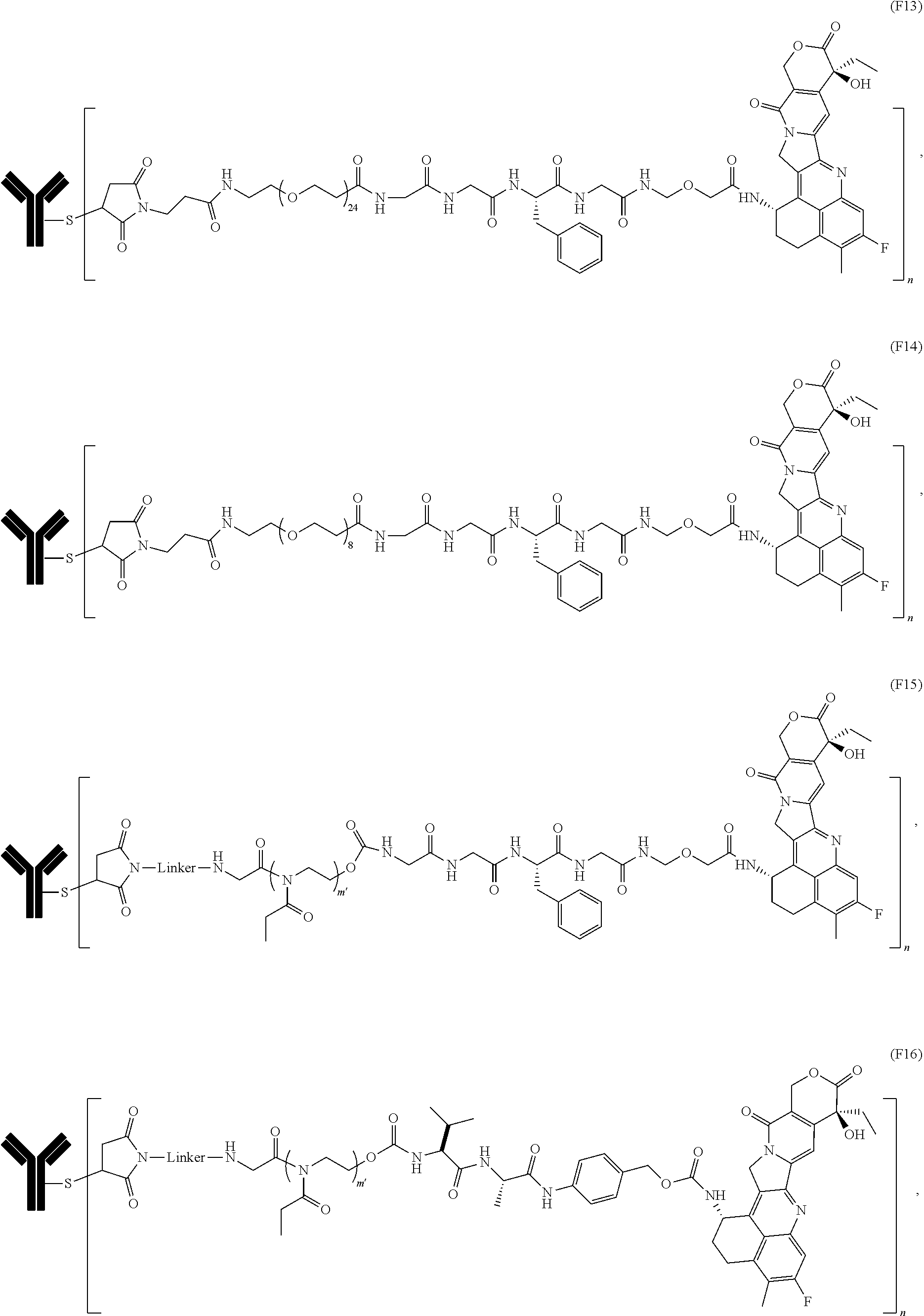

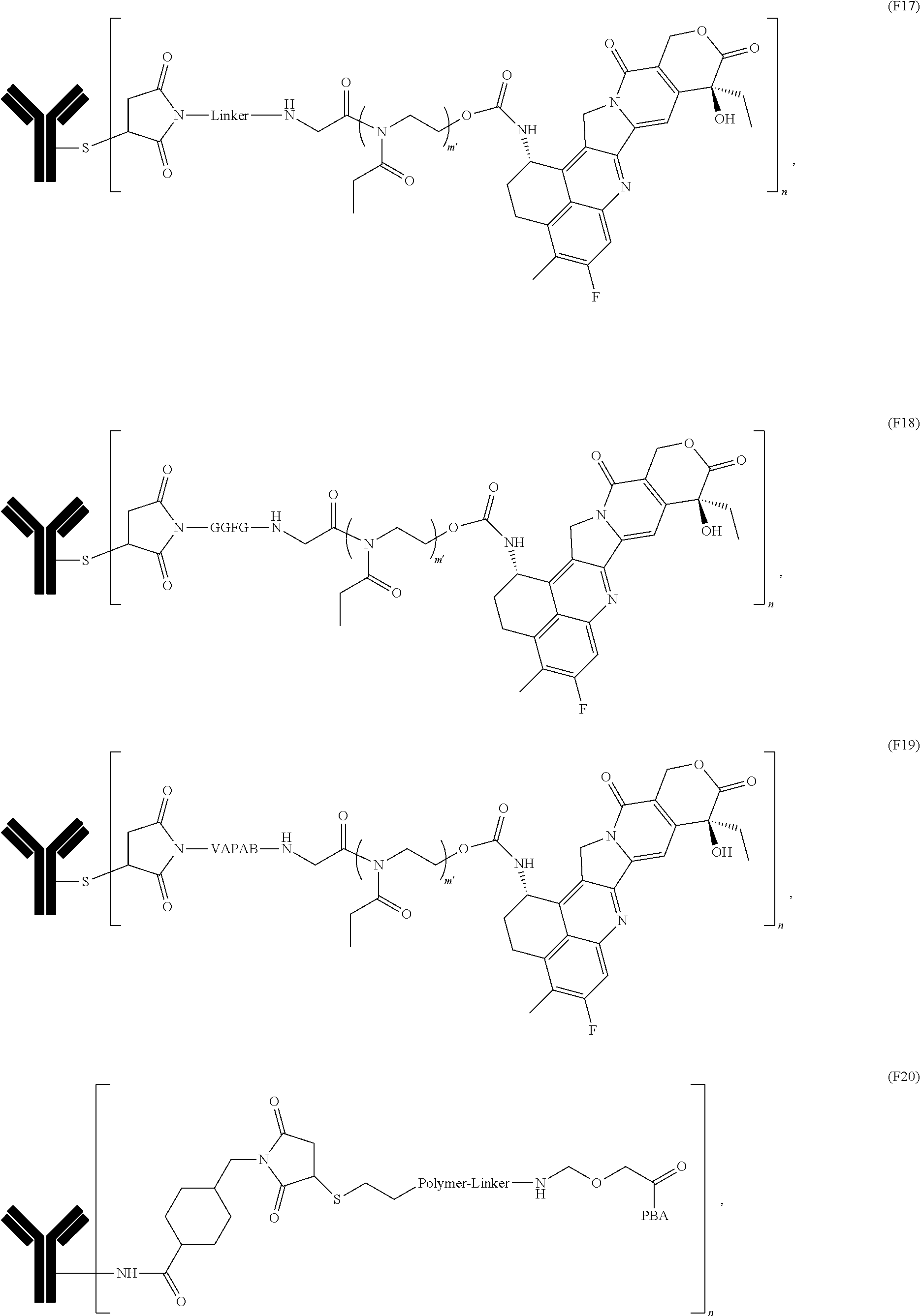

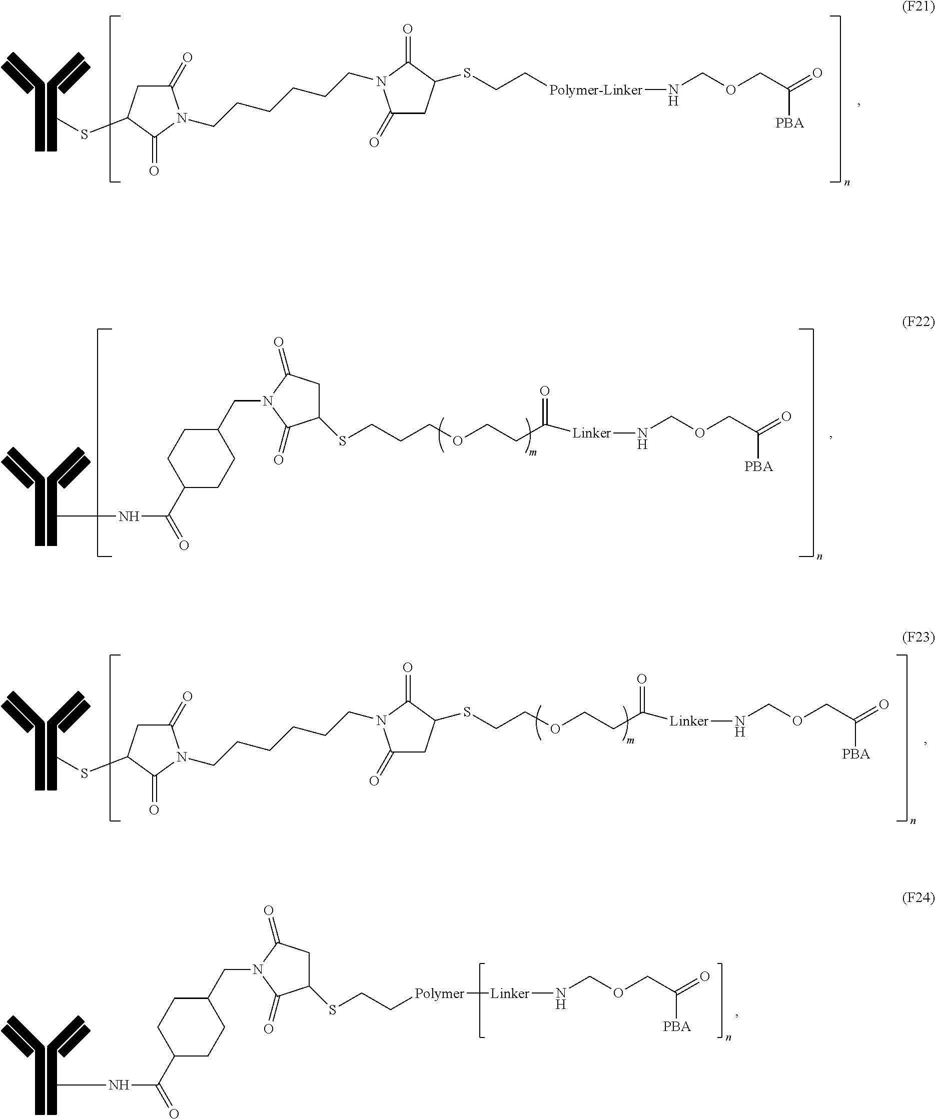

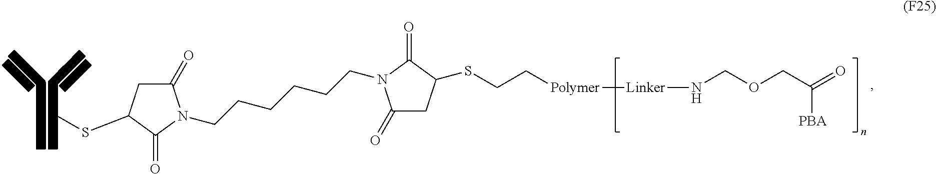

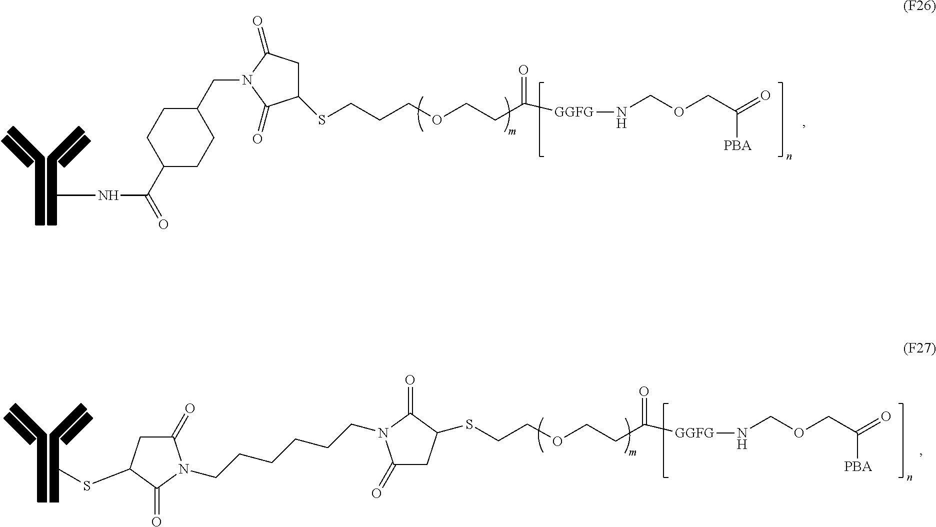

an isomer thereof, an isotope derivative thereof, and a combination thereof,
wherein, m is an integer in a range of from 1-100, m′ is an integer in a range of from 1-100, and n is an integer in a range of from 1 to 100.

The term “PBA” or “Drug” refers to any of the PBA disclosed herein. The term “Exa” refers to exatecan. The term “linker”, “Linker” or “Linker 2” refers to any of the linkers including any of the cleavable linkers disclosed herein. The term “Linker 2-Drug” refers to a linker linked to a drug. The term “GGFG” refers to the GGFG linker. The term “polymer” refers to any of the polymers disclosed herein. The term “PEOx” refers to PEOX polymer. A symbol typically depicting an antibody is used in the formulas throughout this disclosure. Although a typical two-stranded antibody symbol is used in the drawings, the antibody can be an immunoglobin (Ig), IgA, IgD, IgE, IgG, IgM, a part thereof, bispecific antibody, single-chain antibody, a single-chain fragment variables (scFv), any specific antibody disclosed herein, or a combination thereof.

In some cases, n can be an integer in a range of from 1 to 100. In some cases, the pharmaceutical composition disclosed above can comprise a molar ratio of antibody to the drug, i.e., TBA:PBA, in a range of 1:1 to 1:100, 1:2 to 1:100, 1:4 to 1:100, 1:8 to 1:100, 1:12 to 1:100, 1:20 to 1:100, 1:24 to 1:100, 1:48 to 1:100 and 1:64 to 1:100. In some cases, the pharmaceutical composition disclosed above can comprise a molar ratio of antibody to the drug, i.e., TBA:PBA, in a range of from 1:2 to 1:24. In some cases, the pharmaceutical composition disclosed above can comprise a molar ratio of antibody to the drug, i.e., TBA:PBA, in a range of from 1:4 to 1:24. In some cases, the pharmaceutical composition disclosed above can comprise a molar ratio of antibody to the drug, i.e., TBA:PBA, in a range of from 1:8 to 1:24. In some cases, the pharmaceutical composition disclosed above can comprise a molar ratio of antibody to the drug, i.e., TBA:PBA, in a range of from 1:16 to 1:24. One advantage of the ADC disclosed herein is that the number of the drug attached to each antibody can be more than two in one example, more than four in another example, more than 8 in yet another example, more than 10 in yet another example, more than 12 in yet another example, more than 16 in yet another example, and more than 24 in yet another example. It is understood by those skilled in the art that although n is an integer in a range of from 1 to 100, actual measurements of a TBA:PBA ratio can be a number in any of the aforementioned ranges including a fraction due to practical measurement limitations and the potential presence of some un-conjugated TBA, such as un-conjugated antibody.

In some cases, the pharmaceutical composition can comprise one or more PBA, wherein the PBA can comprise at least a compound having a formula selected from (Formula F30 to Formula F42):

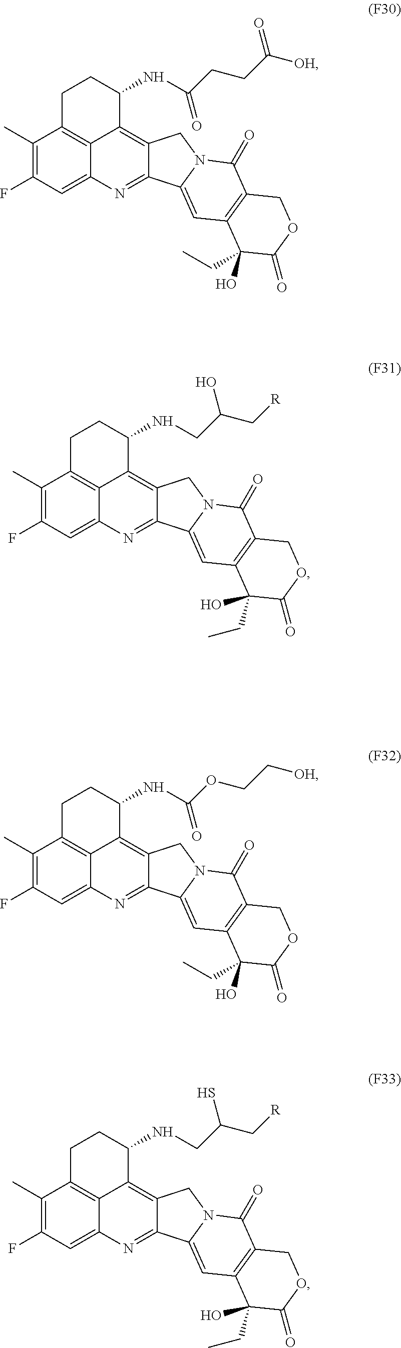

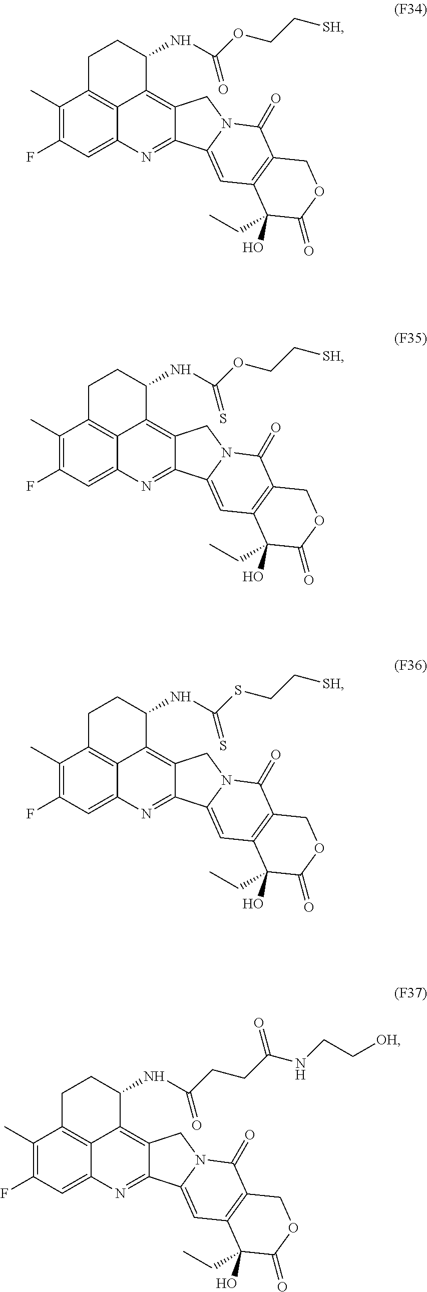 [SMILES: CC[C@@]1(O)C(=O)OCc2c1cc1n(c2=O)Cc2c-1nc1cc(F)c(C)c3c1c2[C@@H](NC(=O)OCCS)CC3 ; CC[C@@]1(O)C(=O)OCc2c1cc1n(c2=O)Cc2c-1nc1cc(F)c(C)c3c1c2[C@@H](NC(=S)OCCS)CC3 ; CC[C@@]1(O)C(=O)OCc2c1cc1n(c2=O)Cc2c-1nc1cc(F)c(C)c3c1c2[C@@H](NC(=S)SCCS)CC3 ; CC[C@@]1(O)C(=O)OCc2c1cc1n(c2=O)Cc2c-1nc1cc(F)c(C)c3c1c2[C@@H](NC(=O)CCC(=O)NCCO)CC3]

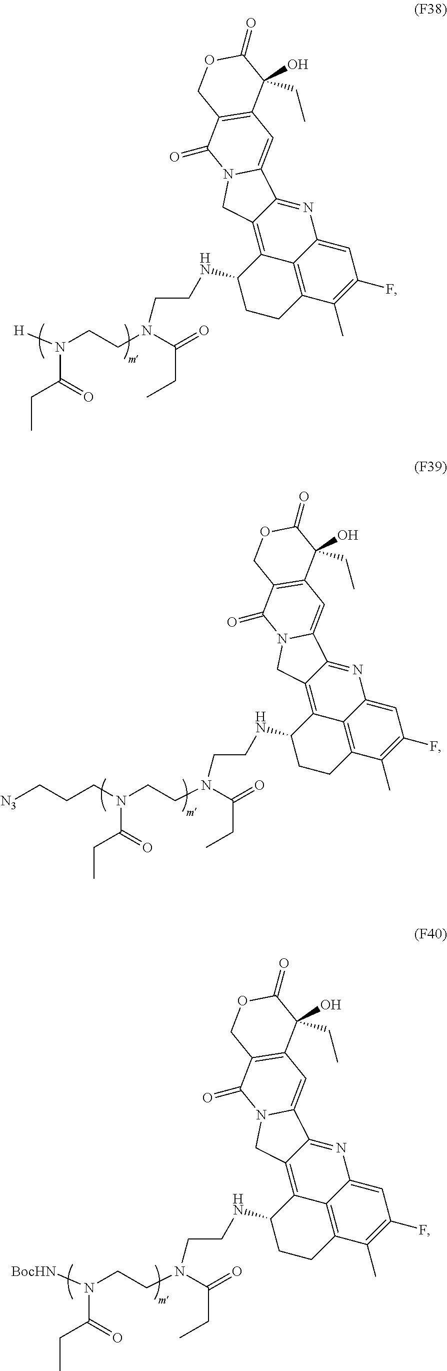

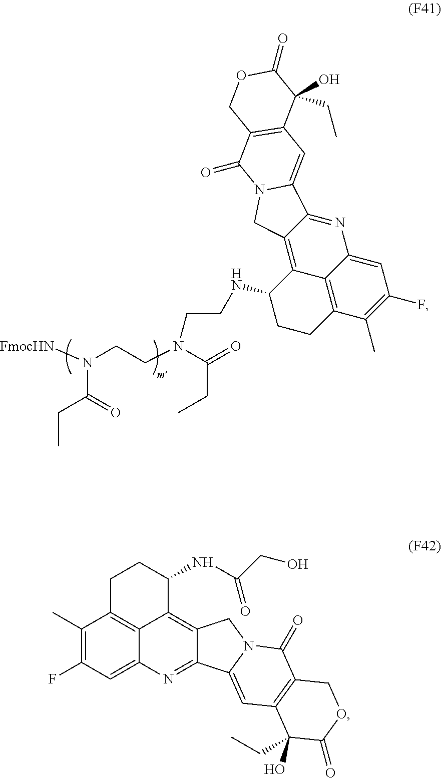

an isomer thereof, an isotope derivative thereof, and a combination thereof, and wherein each the PBA is in a reacted form covalently linked to the TBA, the polymer, or the linker, m′ is an integer in a range of from 1 to 100.

As disclosed herein, the term reacted form refers to a PBA reacted to a polymer, a linker, a coupling molecule, or a combination thereof and covalently linked to a polymer, a linker, a coupling molecule, or a combination thereof.

In some cases, the pharmaceutical composition can comprise a molar ratio of the TBA to the PBA in a range of 1:2 to 1:100.

In some cases, the pharmaceutical composition can comprise one or more of the ADCs disclosed herein and an additional polymer non-covalently associated with the ADC, wherein the additional polymer can comprise a polyoxazoline (POX) that comprises a linear portion, a branched portion, or a combination thereof, and wherein said polyoxazoline (POX) comprises poly(2-methyloxazoline), poly(2-ethyloxazoline), poly(2-propyloxazoline), poly(isopropyloxazoline), or a combination thereof, and wherein the polyoxazoline (POX) can comprise at least one first terminal group modified with H or a hydrophobic moiety and at least one second terminal group modified with a hydrophilic moiety, wherein said first terminal group comprises in a range of from 1% to 99% of H and 1% to 99% of said hydrophobic moiety that comprises saturated or unsaturated aliphatic hydrocarbon having 1 to about 22 carbons, an aromatic hydrocarbon, or a combination thereof, and said second terminal group comprises a group modified by an amine, amide, imine, imide, carboxyl, hydroxyl, ester, ether, acetate, phosphate, ketone, aldehyde, sulfonate, or a combination thereof; or a combination thereof. The polyoxazoline (POX) can comprise oxazoline monomer units in a range of from 1 to 100.

In some cases, the pharmaceutical composition comprises one or more ADCs selected from Formula F2 to Formula F27, or a combination thereof and the aforementioned polyoxazoline (POX).

In some cases, the pharmaceutical composition comprises one or more ADCs comprising the PBA selected from Formula F30 to Formula F42, or a combination thereof and the aforementioned additional polymer comprising polyoxazoline (POX).

In some cases, this disclosure is directed to an antibody-drug conjugate (ADC) comprising:
a targeting bioactive agent (TBA) comprising an antibody or a part thereof having binding affinity to a target molecule that comprises a tumor antigen (Ag);
a payload bioactive agent (PBA) covalently linked to the targeting bioactive agent (TBA) directly or indirectly;
a polymer; and
the ADC having a formula selected from Formula F2 to Formula F27, an isomer thereof, an isotope derivative thereof, and a combination thereof,
wherein, m is an integer in a range of from 1-100, m′ is an integer in a range of from 1-100, and n is an integer in a range of from 1 to 100.

In some cases, the PBA can comprise at least a compound having a formula selected from Formula F30 to Formula F42, an isomer thereof, an isotope derivative thereof, and a combination thereof, and wherein each PBA is in a reacted form covalently linked to the TBA, the polymer, or the linker, m is an integer in a range of from 1 to 100, m′ is an integer in a range of from 1-100.

In some cases, the ADC can comprise a molar ratio of the TBA to the PBA in a range of from about 1:0.01 to about 1:100.

In some cases, the ADC can comprise a molar ratio of the TBA to the PBA in a range of 1:2 to 1:100.

In some cases, this disclosure is further directed to a method for treating or preventing a disease of a subject in need thereof, the method comprising administering the subject with an effective dose of at least a pharmaceutical composition disclosed herein.

In some cases, the pharmaceutical composition can be administered to the subject via intravenous (IV) injection, subcutaneous (SC) injection, intramuscular (IM) injection, intradermal (ID) injection, oral administration, aerosol administration, or a combination thereof.

In some cases, the method disclosed herein can further comprise the step of administering the subject with one or more subsequent bioactive agents selected from a protein, a peptide, an antibody, a fragment of an antibody, a chemical compound, a small molecule drug, one or more chemotherapy drugs, a vaccine, and a combination thereof, wherein each of the one or more subsequent bioactive agents is administered to the subject, prior to, at the same time as, or after administering the pharmaceutical composition.

In some cases, the subsequent bioactive agents can include chemotherapy drugs, paclitaxel, checkpoint inhibitors, Alemtuzumab, alemtuzumab, an antisense nucleic acid, anti-CD19 antibodies, anti-CD20 antibodies, anti-CTLA-4 antibodies, anti-LAG3 antibodies, anti-PD1 antibodies KEYTRUDA® (pembrolizumab), anti-PD-L1 antibodies, anti-TIM-3 antibodies, or a combination thereof.

In some cases, the disease is selected from one or more immune disorders, infectious diseases, cancers, and a combination thereof.

In some cases, the subject is a human patient having a heterogenous tumor.

In some cases, the subject can comprise Ag⁻ tumor cells that are adjacent to the Ag⁺ tumor cells, distal to any Ag⁺ tumor cells, free from contact with any the Ag⁺ tumor cells, or a combination thereof.

In some cases, the pharmaceutical composition can comprise a targeting bioactive agent (TBA) comprising an antibody or a part thereof having binding affinity to Her2, Ag⁺ tumor cytotoxicity to HER2⁺ tumor cells, and Ag⁻ tumor cytotoxicity to HER2⁻ tumor cells.

In some cases, the HER2⁻ tumor cells are adjacent to HER2⁺ tumor cells, distal to HER2⁺ tumor cells, free from contact with any HER2⁺ tumor cells, or a combination thereof.

In some cases, the subject is a human patient having heterogenous HER2⁺ and HER2⁻ breast cancer, metastatic breast cancer, triple-negative breast cancer (TNBC), gastric cancer, colon cancer, or a combination thereof.

In some cases, the method can further comprise the step of administering the subject with one or more immune modulators, wherein each of said one or more immune modulators is administered to the subject simultaneously or sequentially with said pharmaceutical composition.

In some cases, any of the immune modulators disclosed can be suitable. In some cases, the method can comprise administering the subject with one or more immune modulators selected from mTOR, mTOR inhibitor, one or more ATP-competitive mTORC1/2 inhibitors, one or more dual PI3K-mTOR inhibitors, rapamycin, STING (stimulator of interferon genes), STING inhibitors such as C-176, C-170 and C-171; STING activators, such as 3′,3′-cGAMP (3′,3′-cyclic GMP-AMP, Cyclic GMP-AMP, cGAMP); STING agonists such as SR-717 lithium, Alpha-mangostin or diABZI STING agonist (diABZI STING agonist-1, Compound 3), STING agonist-1 (G10), CF501, CF502, CF504, CF505, CF508, CF509, CF510, CF511, or MSA-2; STING antagonist such as SN-011 (GUN35901) or H-151, or a combination thereof.

In some cases, this disclosure is further directed to nanoaggregates comprising a therapeutic bioactive agent having cytotoxicity and a polymer that comprises a polyoxazoline (POX), polyethylene glycol (PEG), or a combination thereof, wherein the polyoxazoline (POX) comprises a linear portion, a branched portion, or a combination thereof, and wherein the polyoxazoline (POX) comprises poly(2-methyloxazoline), poly(2-ethyloxazoline), poly(2-propyloxazoline), poly(2-isopropyloxazoline), poly(2-substituted oxazoline), or a combination thereof, wherein the nanoaggregates have particle sizes in a range of from 1 nm to 140 nm, and the nanoaggregate comprises a tumor cytotoxicity to tumor cells and a non-tumor cytotoxicity to non-tumor cells, wherein the tumor cytotoxicity comprises Ag⁺ tumor cytotoxicity to tumor cells having a tumor antigen (Ag⁺ tumor cells) and Ag⁻ tumor cytotoxicity to tumor cells free from the tumor antigen (Ag⁻ tumor cells), and the nanoaggregates comprise the Ag⁻ tumor cytotoxicity to the Ag⁻ tumor cells in the absence of the Ag⁺ tumor cells.

In some cases, in a range of from 20% to 100% of the nanoaggregates have particle sizes in a range of from 2 nm to 100 nm.

In some cases, in a range of from 40% to 100% of the nanoaggregates have particle sizes in a range of from 2 nm to 80 nm.

In some cases, the therapeutic bioactive agent can comprise a natural or synthetic small molecule-based drug, inorganic-based drug, a toxin molecule having cytotoxicity, a molecule having cytotoxicity to the target cell, a derivative thereof, or a combination thereof.

In some cases, the nanoaggregates disclosed herein can comprise a polymer, wherein the polymer can comprise polyoxazoline (POX) that is non-covalently associated with the payload bioactive agent (PBA) forming nanoaggregates, wherein the polyoxazoline (POX) comprises a linear portion, a branched portion, or a combination thereof, and wherein the polyoxazoline (POX) comprises poly(2-methyloxazoline), poly(2-ethyloxazoline), poly(2-propyloxazoline), poly(isopropyloxazoline), or a combination thereof, and wherein the polyoxazoline (POX) comprises at least one first terminal group modified with H or a hydrophobic moiety and at least one second terminal group modified with a hydrophilic moiety, wherein the first terminal group comprises in a range of from 1% to 99% of H and 1% to 99% of the hydrophobic moiety that comprises saturated or unsaturated aliphatic hydrocarbon having 1 to about 22 carbons, an aromatic hydrocarbon, or a combination thereof, and the second terminal group comprises a group modified by an amine, amide, imine, imide, carboxyl, hydroxyl, ester, ether, acetate, phosphate, ketone, aldehyde, sulfonate, or a combination thereof, or a combination thereof.

In some cases, in a range of from 1% to 100% of the second terminal group is free from primary amine.

In some cases, in a range of from 1% to 100% of the second terminal group comprises hydroxyl group.

In some cases, the nanoaggregate can have a size less than 140 nm before lyophilization. In particular cases, the pharmaceutical composition can comprise nanoaggregate having in a range of from 1 to 140 nm, 1 to 120 nm, 1 to 100 nm, 1 to 80 nm, 1 to 60 nm, 1 to 40 nm, 1 to 20 nm, 1 to 15 nm, 1 to 10 nm, 1 to 8 nm, 1 to 6 nm, 2 to 120 nm, 4 to 120 nm, 4 to 100 nm, 4 to 80 nm, 4 to 60 nm, 4 to 40 nm, 4 to 30 nm, 4 to 20 nm, 4 to 15 nm, before lyophilization. In some cases, the pharmaceutical composition can comprise nanoaggregate having in a range of from 15 to 120 nm, 15 to 100 nm, 15 to 80 nm, 15 to 60 nm, 15 to 40 nm, 15 to 30 nm, 15 to 20 nm, 10 to 120 nm, 10 to 100 nm, 10 to 80 nm, 10 to 60 nm, 10 to 40 nm, 10 to 30 nm, 10 to 20 nm, 5 to 120 nm, 5 to 100 nm, 5 to 80 nm, 5 to 60 nm, 5 to 40 nm, 5 to 30 nm, 5 to 20 nm, 5 to 10 nm, 2 to 120 nm, 2 to 100 nm, 2 to 80 nm, 2 to 60 nm, 2 to 40 nm, 2 to 30 nm, 2 to 20 nm, 2 to 15 nm, 2 to 10 nm, and 2 to 5 nm, before lyophilization. The size of nanoaggregate can be measured using a dynamic light scattering (DLS) method. Other methods known to those skilled in the art can also be used.

In some cases, the nanoaggregates disclosed herein can further comprise a targeting bioactive agent (TBA), wherein the targeting bioactive agent (TBA) comprises a peptide, a protein, DNA, RNA, nucleic acids, oligo nucleic acids, mRNA, siRNA, sgRNA, a monoclonal antibody, a fragment of a monoclonal antibody, a polyclonal antibody, a fragment of a polyclonal antibody, a synthetic antibody, a fragment of a synthetic antibody, antigen-binding portions thereof, a ligand of cell surface receptor, an agonist, an activator, an inhibitor, an antagonist, a naked nucleic acid, a nucleic acid, a ribozyme, a virus, a virus-like particles and a combination thereof, Alemtuzumab, alemtuzumab, an antisense nucleic acid, anti-CD19 antibodies, anti-CD20 antibodies, anti-CTLA-4 (cytotoxic T lymphocyte-associated antigen) antibodies, anti-LAG3 (lymphocyte activation gene-3) antibodies, anti-PD1 antibodies KEYTRUDA® (pembrolizumab), anti-PD-L1 antibodies, anti-TIM-3 (T cell immunoglobulin and mucin domain-3) antibodies, Atezolizumab, BAVENCIO® (avelumab), Bevacizumab, bevacizumab, Blinatumomab, Brentuximab, Cetuximab, cetuximab, chimeric antigen receptor or chimeric antigen T-cell receptor (CAR-T), cytokines, Daratumumab, Denosumab, Dinutuximab, Durvalumab, Elotuzumab, gemtuzumab, granulocyte colony stimulating factor (G-CSF), Ibritumomab, IMFINZI® (durvalumab), Inotuzumab, interferon α, interferon α2a, interleukins, Ipilimumab, lectins, Necitumumab, Neupogen (Filgrastim), Obinutuzumab, Ofatumumab, Olaratumab, OPDIVO® (nivolumab), Panitumumab, Pembrolizumab, Pertuzumab (anti-HER2), Ramucirumab, Rituximab, rituximab, Siltuximab, Tislelizumab (BGB-A317), Toripalimab, siRNAs, T-cell receptor (TCR), TECENTRIQ® (atezolizumab), tositumomab, trastuzumab (anti-HER2), or a combination thereof.

In some cases, the nanoaggregates disclosed herein can comprise one or more PBAs disclosed herein. In some cases, the nanoaggregates disclosed herein can comprise one or more PBAs selected from the Formula F30 to Formula F42. In some cases, the nanoaggregates disclosed herein can comprise one or more ADCs selected from Formula F2 to Formula F27.

In some cases, this disclosure is further directed to a use of aNano-ADC for manufacturing of a medicament for treating or preventing a disease, wherein the Nano-ADC comprises:
a targeting bioactive agent (TBA) comprising an antibody or a part thereof having binding affinity to a target molecule that comprises a tumor antigen (Ag);
a payload bioactive agent (PBA) covalently linked to the targeting bioactive agent (TBA) directly or indirectly;
optionally, a polymer; and
optionally, a pharmaceutical suitable carrier;
wherein the polymer, when present, is covalently or non-covalently bond to the targeting bioactive agent (TBA) and the payload bioactive agent (PBA);
wherein the polymer comprises polyoxazoline (POX), a polyethylene glycol (PEG), or a combination thereof, wherein the polyoxazoline (POX) comprises a linear portion, a branched portion, or a combination thereof, and wherein the polyoxazoline (POX) comprises poly(2-methyloxazoline), poly(2-ethyloxazoline), poly(2-propyloxazoline), poly(2-isopropyloxazoline), poly(2-substituted oxazoline), or a combination thereof.
wherein the targeting bioactive agent (TBA), payload bioactive agent (PBA), and the polymer are aggregated to form nanoaggregates having sizes in a range of from 1 nm to 140 nm; and
wherein the pharmaceutical composition comprises a tumor cytotoxicity to tumor cells and a non-tumor cytotoxicity to non-tumor cells, wherein the tumor cytotoxicity comprises Ag⁺ tumor cytotoxicity to tumor cells having the tumor antigen (Ag⁺ tumor cells) and Ag⁻ tumor cytotoxicity to tumor cells free from the tumor antigen (Ag⁻ tumor cells).

In some cases, the disease can be selected from one or more immune disorders, infectious diseases, cancers, and a combination thereof.

In some cases, the pharmaceutical composition or the nanoaggregates can comprise a targeting bioactive agent (TBA) that comprises a member of a small molecule binding pair, wherein the small molecule binding pair can comprise a member being a small molecule and a member of either a small or a large molecule. Members of such a binding pair can bind to each other under proper conditions. Examples of small molecule binding pairs can include inhibitor-target binding pair, stimulator-effector binding pair, biotin-streptavidin binding pair, avidin-streptavidin/neutravidin binding pair, dinitrophenol (DNP) and an anti-DNP antibody binding pair, a digoxin and an anti-digoxin antibody binding pair, a digoxigenin and an anti-digoxigenin antibody binding pair, a hapten and an anti-hapten antibody binding pair, a polysaccharide and a polysaccharide binding moiety, such as a lectin, or a combination thereof. With such a member of a small molecule binding pair as a TBA, a payload bioactive agent (PBA) can be delivered to a specific target, such as a set of target cells or tissues.

It is understood by those skilled in the art that although n is an integer in a range of from 1 to 100, actual measurements of a TBA:PBA ratio can be a number in any of the aforementioned ranges including a fraction due to practical measurement limitations and/or the potential presence of some un-conjugated TBA, such as un-conjugated antibody. As disclosed above, the pharmaceutical composition can comprise a measured TBA:PBA in a range of from 1:0.01 to 1:100. In some cases, the pharmaceutical composition of this disclosure can comprise free antibody that is not conjugated with a payload bioactive agent (PBA), as disclosure in detail hereafter. In some cases, the pharmaceutical composition of this disclosure can a Drug-to-Antibody Ratio (DAR), i.e., PBA:TBA molar ratio, in a range of 1:100 to 1:0.01.

A symbol typically depicting an antibody is used in the formulas throughout this disclosure. Although a typical two-stranded antibody symbol is used in the drawings, the antibody can be an immunoglobin (Ig), IgA, IgD, IgE, IgG, IgM, a part thereof, or a combination thereof. The antibody can be a typical two chain antibody having heavy chains and light chains arranged in three portions (or regions) in a Y shape, a two heavy chain antibody that lacks light chains, a single chain antibody, a multi-valent antibody that can comprise binding sites having affinities to 2 or more targeting sites or multiple antigen binding affinities, a part thereof (i.e., a fragment thereof), or a combination thereof.

The polymer, the PBA, or the linker can be attached to the antibody, also referred to as conjugated to the antibody, via S— bond (such as thiol-bond or thiol conjugation, via cysteine conjugation), N— bond (such as via lysine conjugation), glycol bond, modified glycol bond, GlcNAc (N-acetylglucosamine), substituted GlcNAc, GlcNAc-galactose, GlcNAc-mannose, GlcNAc-glucose, GlcNAc-glucuronic acid, GlcNAc-fucose, GlcNAc-N-acetylneuraminic acid, site specific bond, random bond, or any other bonds or conjugations known to or developed by those skilled in the art. Bioconjugation to other amino acid residues of the antibody, such as tyrosine (Tyr), tryptophan (Trp), and methionine (Met) can also be suitable. The S— bond conjugation can include cysteine conjugations, such as reactions via maleimide, for example, with a MC linker, a MC-VC-PABC linker, MC-VC-PAB-NH₂ linker, a MC-GGFG-AM, a CL2A linker, and maleimide-VA-PABC linker; alkynyl carboxylic acid derivatives; 5-methylene pyrrolone (5MP); 5,5′-dithiobis-(2-nitrobenzoic acid) (DTNB); phenyloxadiazole sulfone (PODS); cyclooctyne, an alkyne moiety in aza-dibenzocyclooctyne (DBCO) reagent that is shown to react with a cysteine residue as a side reaction; via 3-arylpropionitrile (APN); perfluoroarene; ethynylbenziodoxolone (EBX); bicyclo[1.1.0]butane (BCB) carboxylic amide; allenamide; and disulfide functionalization such as with a bis-reactive linker that undergoes reactions with both thiol residues (also known as disulfide rebridging) to produce an ADC having a controlled DAR when modifying antibodies with four reducible interchain disulfides. Lysine conjugation can include N-hydroxysuccinimide (NHS) ester, isocyanates and isothiocyanates, 4-azidobenzoyl fluoride (ABF), β-lactams, Phospha-Mannich, α,β-unsaturated sulfonamide, sulfonyl acrylate, and others. Tyrosine conjugation can include Mannich-type three component reactions, diazonium salt reactions, 4-phenyl-3H-1,2,4-triazole-3,5(4H)-dione (PTAD), phenothiazine, and other reactions. Tryptophan conjugation can include 9-azabicyclo [3.3.1]nonane-3-one-N-oxyl (keto-ABNO), N-substituted pyridinium salts, and other reactions. Methionine conjugation can include oxaziridine, hypervalent iodine, lumiflavin-catalysis, and others. Reactions described by Kang et al., in Chemical Science (12, 13613-13647, October 2021, DOI: 10.1039/D1SC02973H) and U.S. Pat. No. 9,504,758, granted on Nov. 29, 2016, can be suitable. In some cases, antibodies having genetically engineered non-natural amino acids (NNAA), such as p-acetylphenylalanine and p-azidomethylphenyl alanine, can also be suitable for producing antibody-drug conjugates. In some cases, the pharmaceutical composition can comprise a Nano-ADC that can comprise an antibody having genetically engineered non-natural amino acids (NNAA), such as p-acetylphenylalanine and p-azidomethylphenyl alanine, enzymatic conjugations such as those produced with bacterial transglutaminase (TGase), Sortase, Glycosyl transferase, Formylglycine-generating enzyme (FGE), or Isoprenoid transferase, or a combination thereof.

The linkers L1 and L2 can be the same or different. The linkers L1 and L2 can comprise the same or different cleavable linkers.

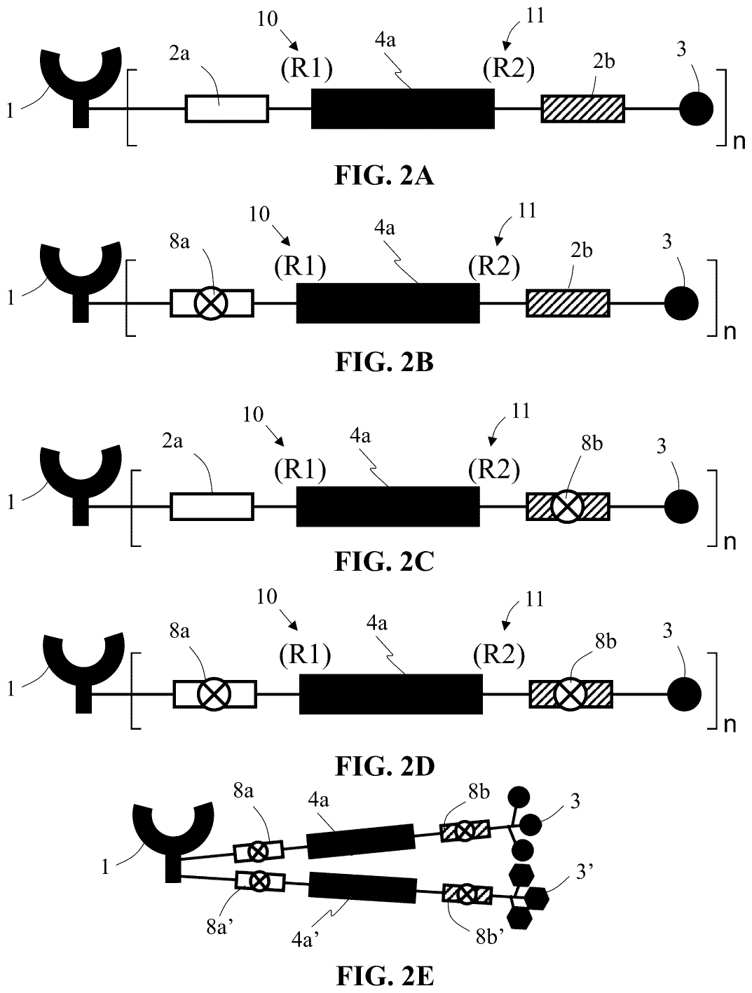

Further schematic illustrations of some representative examples of the pharmaceutical composition having a polymer (4a) covalently linked to a targeting bioactive agent (TBA) (1) and a payload bioactive agent (PBA) (3) are shown in FIG. 2A-FIG. 2E. In some cases, the pharmaceutical composition can comprise linker L1 (2a) and L2 (2b) without any cleavable linkers (FIG. 2A). In some cases, the pharmaceutical composition can comprise at least one L1 cleavable linker (8a) in the linker L1 (2a) position between the TBA (1) and the polymer (4a) (FIG. 2B). In some cases, the pharmaceutical composition can comprise at least one L2 cleavable linker (8b) in the linker L2 position between the PBA (3) and the polymer (4a) (FIG. 2C). In some cases, the pharmaceutical composition can comprise at least one L1 cleavable linker (8a) in the linker L1 position between the TBA (1) and the polymer (4a) and at least one L2 cleavable linker (8b) in the linker L2 position between the PBA (3) and the polymer (4a) (FIG. 2D). In some cases, the pharmaceutical composition can comprise at least two same or different PBAs, such a first PAB and a second PBA (FIG. 2E). In some cases, the pharmaceutical composition can comprise an ADC (i.e., a Nano-ADC disclosed herein) that can comprise at least a first PBA, such as the PBA (3) disclosed earlier covalently linked to a first polymer, such the polymer (4a) and a second PBA (3′) covalently linked to a second polymer (4a′), wherein the first polymer and the second polymer each is covalently linked to a TBA (1), such as an antibody, wherein the first polymer and the second polymer can be the same or different, the first PBA and the second PAB can be the same or different and each of the at least two different PBAs can be selected from same or different natural or synthetic small molecule-based drugs, inorganic-based drugs, toxin molecules having cytotoxicity, molecules having cytotoxicity to target cells, derivatives thereof, or a combination thereof. The ADC can comprise one or more cleavable linkers, such as a first cleavable linker that can be the L1 cleavable linker (8a), a second cleavable linker (8a′), a third linker that can be the L2 cleavable linker (8b) and a 4^{th} cleavable linker (8b′) as schematically shown in FIG. 2E. The first cleavable linker, the L2 cleavable linker, the L2 cleavable linker and the 4^{th} linker can be the same or different. Although examples in FIG. 2D-FIG. 2E illustrate ADCs having linkers in the L1 and L2 positions, an ADC can have only one of the L1 and L2 linkers. In some cases, the ADC can comprise only one linker in the L1 position, such as the L1 cleavable linker (8a) or the second cleavable linker (8a′). In some cases, an ADC can have only one of the L1 and L2 linkers. In some cases, the ADC can comprise only one linker in the L2 position, such as the L2 cleavable linker (8b) or the 4th cleavable linker (8a′). The linkers in the L1 and the L2 positions can also be non-cleavable.

R1 (10) and R2 (11) represent the reacted first terminal group and the reacted second terminal group, respectively. The polymers (4a, 4b, 4c) can be a linear polymer, asymmetric branched polymer, symmetric branched polymer, or a combination thereof. Legends: Open rectangle, linker L1; Hatched rectangle, linker L2; Solid rectangle, the polymer of this disclosure that can be a linear polymer, asymmetric branched polymer, symmetric branched polymer, or a combination thereof; Crossed circle, cleavable bond; Solid circle, payload bioactive agent (PBA).

Throughout this disclosure, each TBA can be covalently linked to one or more PBAs, n is an integer and can be in a range of from 1 to 100.

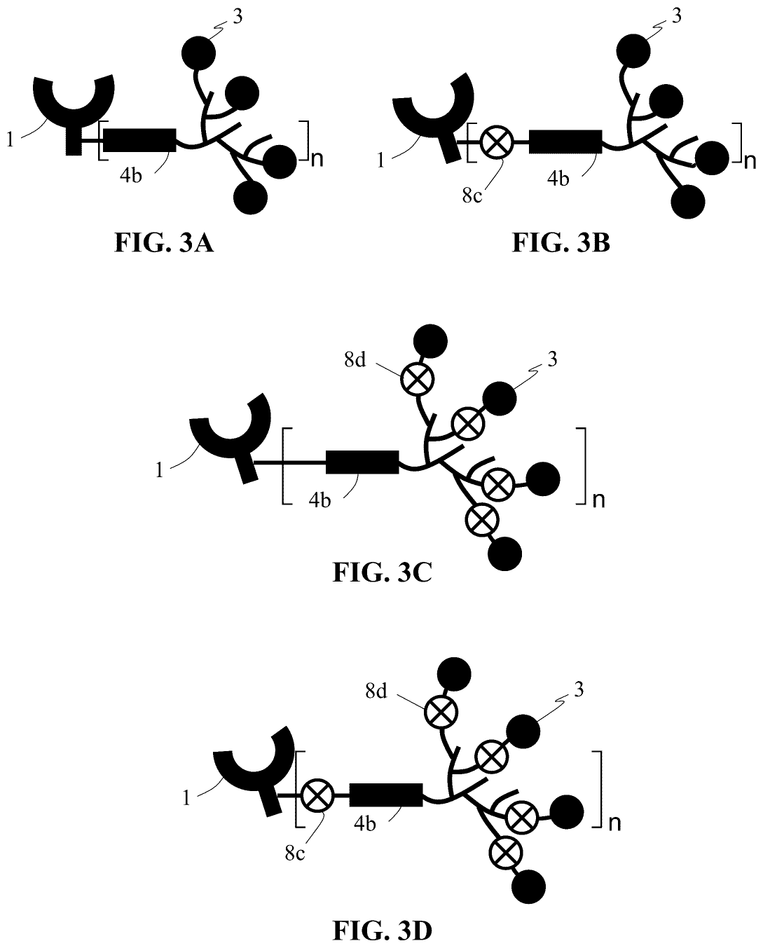

Some examples of the pharmaceutical composition comprising a polymer having asymmetrical branches are shown in FIG. 3A-FIG. 3D. In some cases, a pharmaceutical composition can comprise a targeting bioactive agent (TBA) (1) covalently linked to a polymer having asymmetrical branches (4b) that is covalently linked to at least one payload bioactive agent (PBA) (3) (FIG. 3A) without any cleavable linker. In some cases, a pharmaceutical composition can comprise a targeting bioactive agent (TBA) (1) covalently linked to a polymer having asymmetrical branches (4b) that is covalently linked to at least one payload bioactive agent (PBA) (3) and at least one cleavable linker (8c) between the TBA (1) and polymer (4b) (FIG. 3B). In some cases, a pharmaceutical composition can comprise a targeting bioactive agent (TBA) (1) covalently linked to a polymer having asymmetrical branches (4b) that is covalently linked to at least one payload bioactive agent (PBA) (3) and at least one cleavable linker (8d) between the polymer (4b) and at least one of the payload bioactive agents (PBA) (3) (FIG. 3C). In some cases, a pharmaceutical composition can comprise a targeting bioactive agent (TBA) (1) covalently linked to a polymer having asymmetrical branches (4b) that is covalently linked to at least one payload bioactive agent (PBA) (3), at least one cleavable linker (8c) between the TBA (1) and polymer (4b) and at least one cleavable linker (8d) between the polymer (4b) and at least one of the payload bioactive agent (PBA) (3) (FIG. 3D).

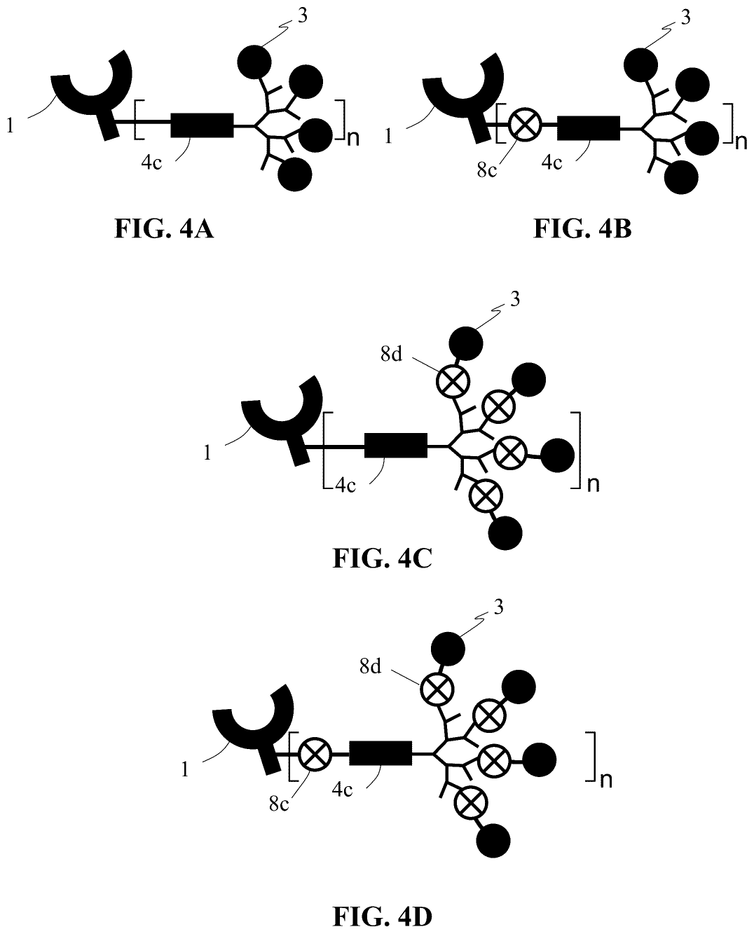

Some examples of the pharmaceutical composition comprising a polymer having symmetrical branches are shown in FIG. 4A-FIG. 4D. In some cases, the pharmaceutical composition can comprise a targeting bioactive agent (TBA) (1) covalently linked to a polymer having symmetrical branches (4c) that is covalently linked to at least one payload bioactive agent (PBA) (3) (FIG. 4A) without any cleavable linker. In some cases, the pharmaceutical composition can comprise a targeting bioactive agent (TBA) (1) covalently linked to a polymer having symmetrical branches (4c) that is covalently linked to at least one payload bioactive agent (PBA) (3) and at least one cleavable linker (8c) between the TBA (1) and polymer (4c) (FIG. 4B). In some cases, the pharmaceutical composition can comprise a targeting bioactive agent (TBA) (1) covalently linked to a polymer having symmetrical branches (4c) that is covalently linked to at least one payload bioactive agent (PBA) (3) and at least one cleavable linker (8d) between the polymer (4c) and at least one of the payload bioactive agents (PBA) (3) (FIG. 4C). In some cases, the pharmaceutical composition can comprise a targeting bioactive agent (TBA) (1) covalently linked to a polymer having symmetrical branches (4c) that is covalently linked to at least one payload bioactive agent (PBA) (3), at least one cleavable linker (8c) between the TBA (1) and polymer (4c) and at least one cleavable linker (8d) between the polymer (4c) and at least one of the payload bioactive agent (PBA) (3) (FIG. 4D).

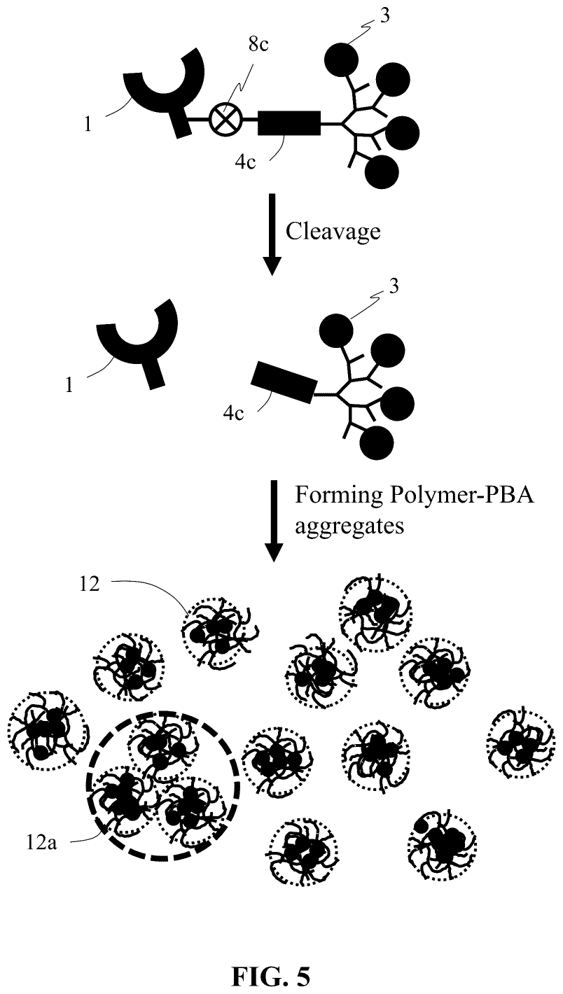

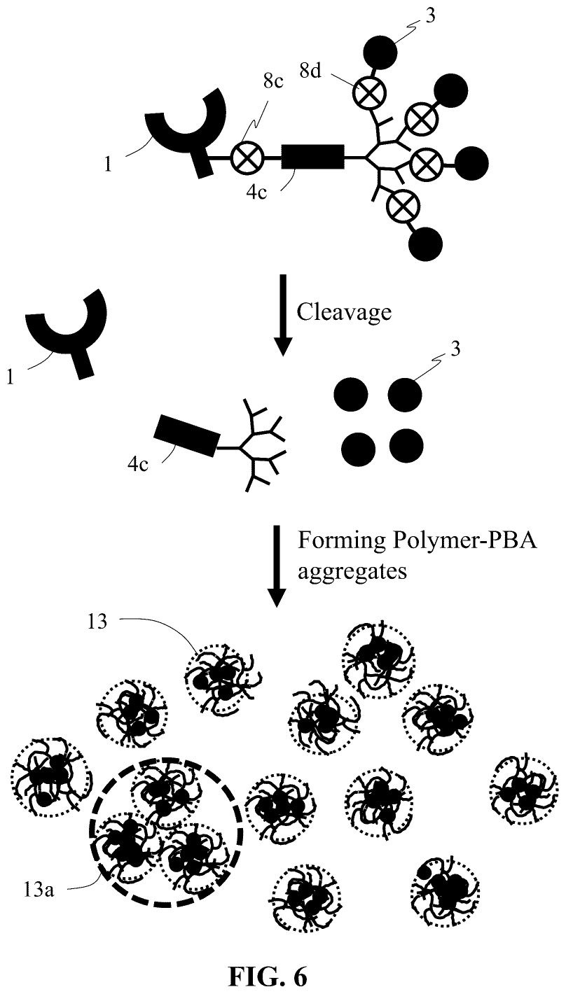

In some cases, the pharmaceutical composition can comprise one cleavable linker between the targeting bioactive agent (TBA) and the polymer. Once the pharmaceutical composition is subject to a proper cleavage condition, such as reducing agent for a disulfide bond linker, acidic condition in lysosome for an acid sensitive linker, an enzyme for a peptide linker, or other conditions, the cleavable linker can be cleaved resulting in the dissociation of the TBA from the polymer-PBA complex. The polymer-PBA can form polymer-PBA aggregates in aqueous environments such as in blood stream, in plasma, or in cytosol (FIG. 5). In some cases, a payload bioactive agent (PBA) can form nanoaggregates that comprise the polymer and the PBA, wherein the polymer and the PBA are linked covalently forming individual nanoaggregates (12) or nanoaggregates complex (12a). In some cases, the pharmaceutical composition can comprise one cleavable linker between the targeting bioactive agent (TBA) and the polymer and at least one cleavable linker between the polymer and the PBA. Once the pharmaceutical composition is subject to a proper cleavage condition in vivo or in vitro, the cleavable linker can be cleaved resulting in the dissociation of the TBA from the pharmaceutical composition, polymer and PBA. The dissociated polymer and PBA can form polymer-PBA aggregates in aqueous environments such as in blood stream, in plasma, or in cytosol (FIG. 6). In some cases, a payload bioactive agent (PBA) can form nanoaggregates that comprise the polymer and the PBA, wherein the polymer and the PBA are aggregated non-covalently forming individual nanoaggregates (13) or nanoaggregates complex (13a). The dissociated polymer and PBA can form polymer-PBA aggregates that are free from the TBA.

In some cases, this disclosure is further directed to a polymer-PBA aggregate comprising the polymer and the PBA disclosed herein and wherein the polymer-PBA aggregate is free from the TBA.

In some cases, the polymer-PBA aggregate can be produced by dissociating the TBA from the pharmaceutical composition in vivo or in vitro. In some cases, the polymer-PBA aggregate can be produced by cleaving one or more of the cleavable linkers in vivo or in vitro.

In some cases, functionalized PEOX polymer can facilitate the formation of polymer-PBA-albumin aggregates in vitro, in blood stream, in plasma, or in cytosol. In some cases, the polymer can comprise polyoxazoline (POX) that comprises a linear portion, a branched portion, or a combination thereof, and wherein said polyoxazoline (POX) comprises poly(2-methyloxazoline), poly(2-ethyloxazoline), poly(2-propyloxazoline), poly(isopropyloxazoline), or a combination thereof, and wherein the polyoxazoline (POX) comprises at least one first terminal group modified with H or a hydrophobic moiety and at least one second terminal group modified with a hydrophilic moiety, wherein said first terminal group comprises in a range of from 1% to 99% of H and 1% to 99% of said hydrophobic moiety that comprises saturated or unsaturated aliphatic hydrocarbon having 1 to about 22 carbons, an aromatic hydrocarbon, or a combination thereof, and said second terminal group comprises a group modified by an amine, amide, imine, imide, carboxyl, hydroxyl, ester, ether, acetate, phosphate, ketone, aldehyde, sulfonate, or a combination thereof; or a combination thereof.

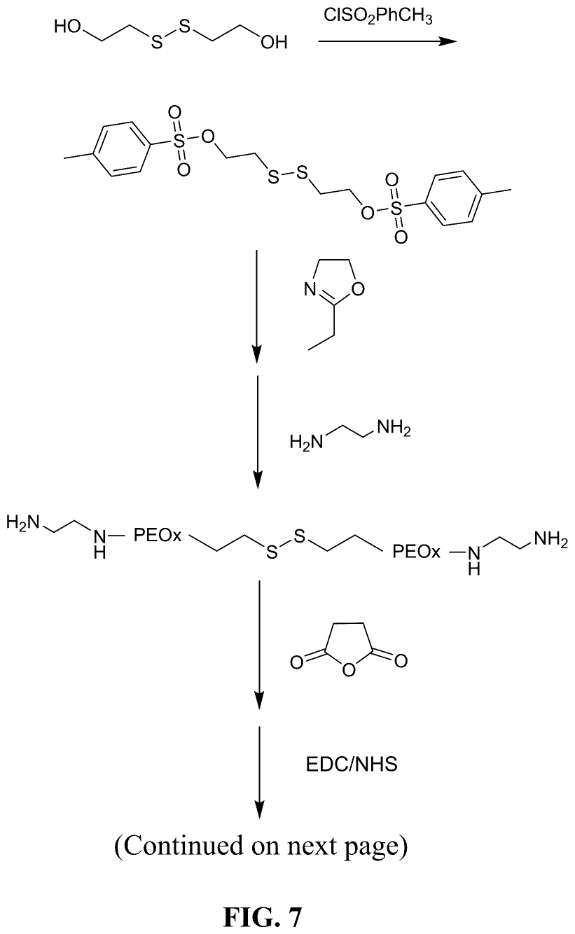

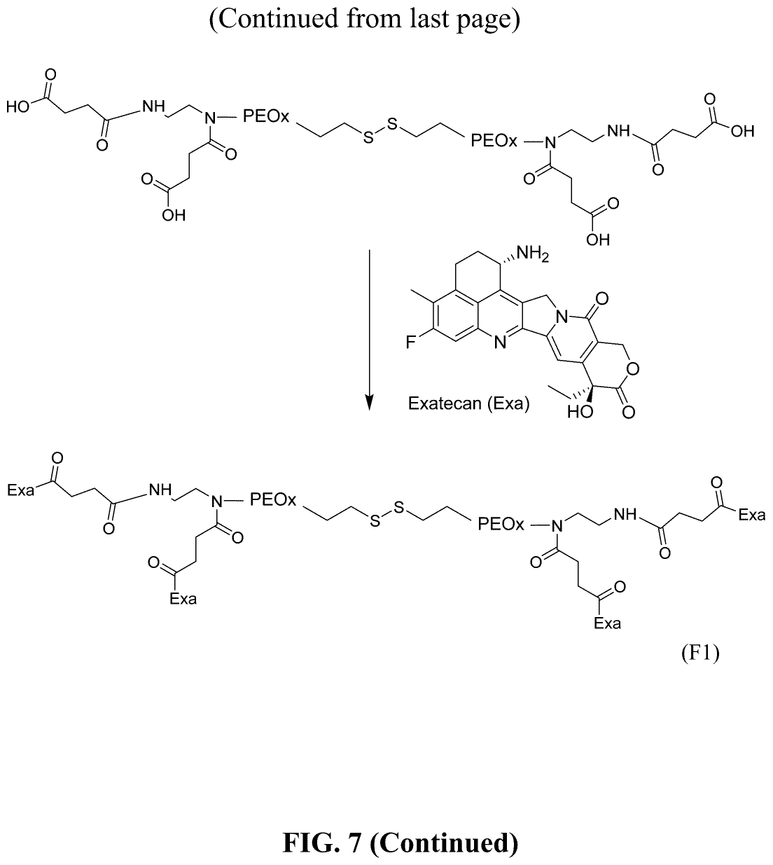

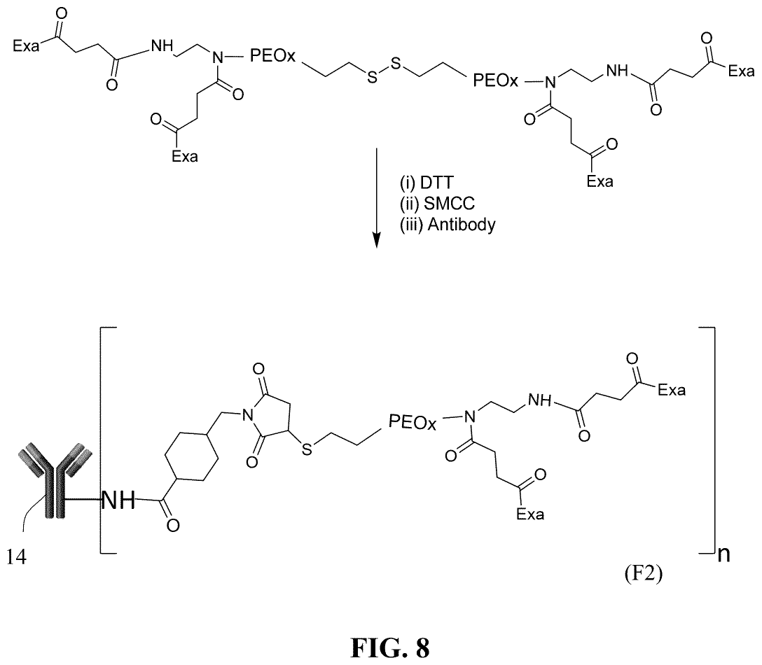

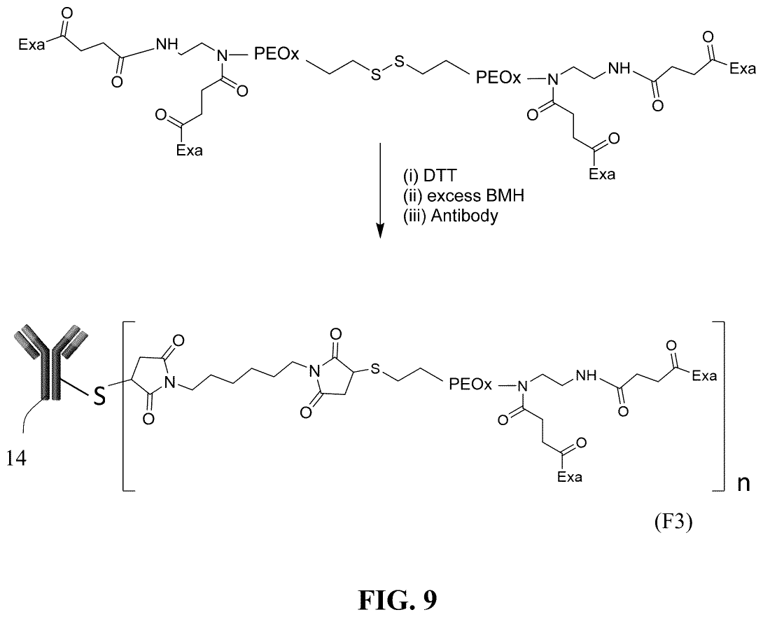

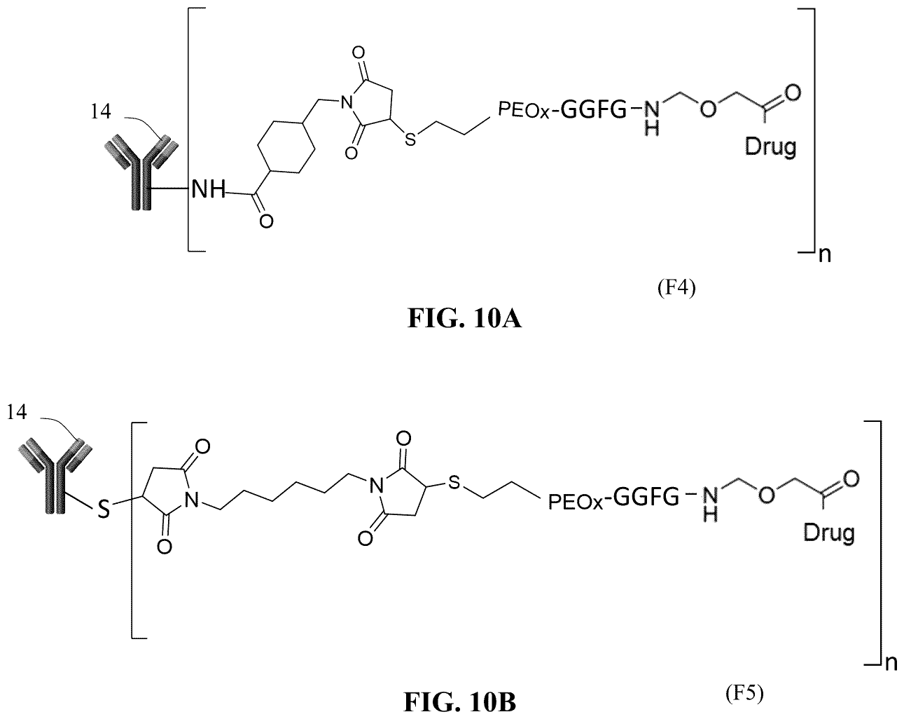

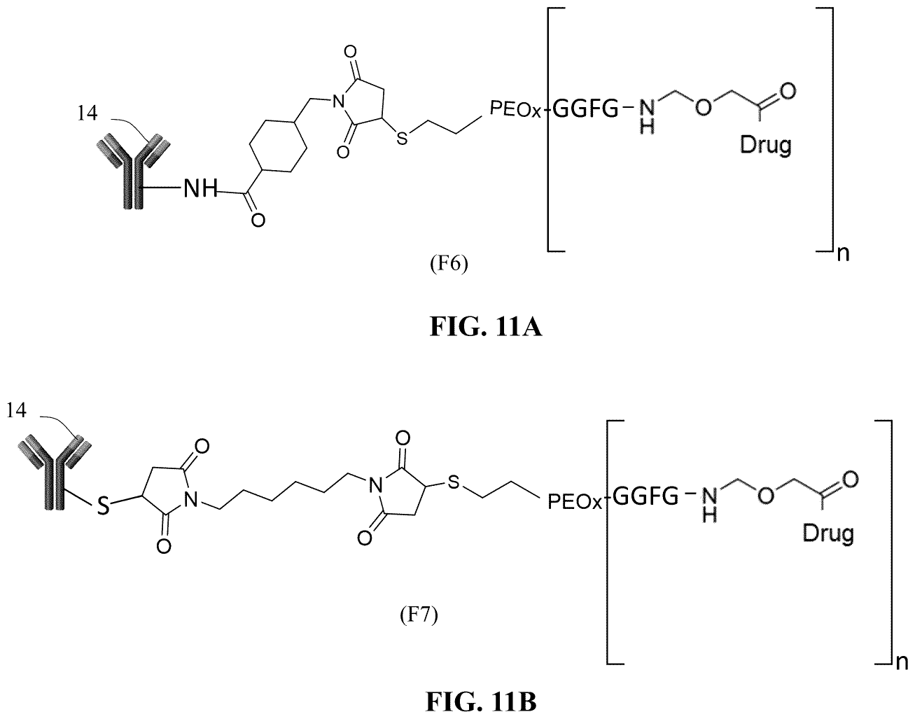

In some cases, the PEOX polymer can be functionalized to comprise disulfide bonds and covalent linkage of multiple drug molecules, such as two or more exatecan (Exa) to the polymer (FIG. 7). The functionalized PEOX polymer can be linked to a TBA, such as an antibody (14) to form an ADC, such as shown with Formula F2 with an —N— linkage, such as via a lysine (FIG. 8) or with an —S— linkage, such as via a cysteine (FIG. 9). Reaction schemes are schematically shown in FIG. 7, FIG. 8 and FIG. 9. Further examples of ADCs formed with PEOX polymers can include Formula F4 (FIG. 10A), Formula F5 (FIG. 10B), Formula F6 (FIG. 11A), Formula F7 (FIG. 11B).

In some cases, the PEOX polymer can be functionalized to have one or more desired functional groups as the first terminal group and the second terminal group, respectively. In some cases, a functionalized PEOX polymer can comprise one or more end groups (EG) (INT-1-INT-7) (FIG. 12). In some cases, a functionalized PEOX polymer can comprise one or more end groups (EG) —H, —NH₂, —CH₂N₃, —CH₂CH₂N₃, —CH₂CH₂CH₂N₃, —NHBoc, —NHFmoc, or a combination thereof. In some cases, a functionalized PEOX polymer can comprise one or more end groups (EG) selected from the group consistent of —H, —NH₂, —NHCH₂NH₃, —NHCH₂CH₂NH₃, —NHCH₂CH₂CH₂NH₃, —CH₂N₃, —CH₂CH₂N₃, —CH₂CH₂CH₂N₃, —NHBoc, —NHFmoc, —OH, —N₃, and a combination thereof. In some cases, a functionalized PEOX polymer can comprise a first terminal group comprising one or more end groups (EG) selected from —H, —NH₂, —NHCH₂NH₃, —NHCH₂CH₂NH₃, —NHCH₂CH₂CH₂NH₃, —CH₂N₃, —CH₂CH₂N₃, —CH₂CH₂CH₂N₃ and a combination thereof. In some cases, a functionalized PEOX polymer can comprise a second terminal group selected from —NHBoc, —NHFmoc, —OH, —N₃, and a combination thereof. In some cases, a functionalized PEOX polymer can comprise one or more end groups (EG) having a formula

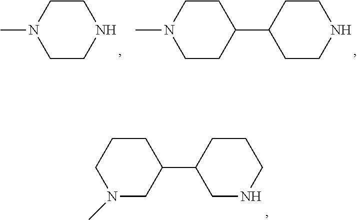

or a combination thereof. Some examples are schematically shown as INT-5, INT-6 and INT-7 (FIG. 12). As disclosed herein, isomers and an isotope derivative thereof, or a combination thereof, can also be suitable.

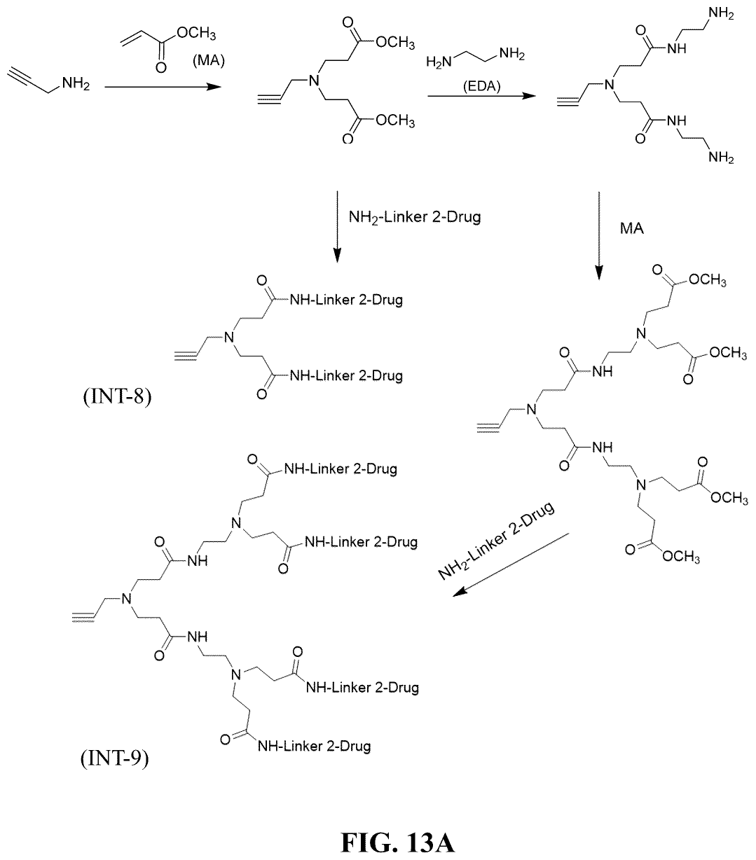

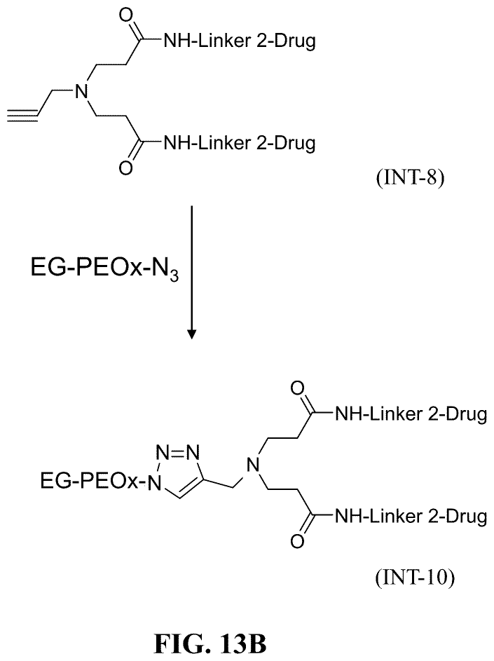

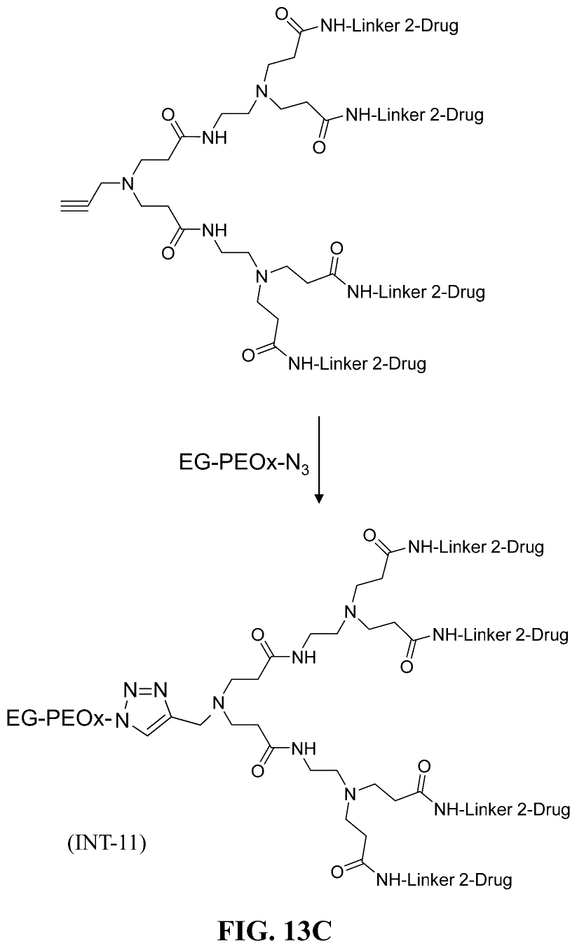

In some cases, multiple PBAs, such as drug molecules, can be linked to linkers, such as one or more linkers L2 and then linked to a functionalized PEOX polymer via reaction scheme disclosed in FIG. 13A-FIG. 13C. In some cases, a functionalized PEOX can comprise two PBAs, such as Exatecan, linked to one PEOX polymer at one or more of the second terminals (INT-8) (FIG. 13A) and INT-10 (FIG. 13B). In some cases, a functionalized PEOX can comprise four PBAs, such as Exatecan, linked to one PEOX polymer at one or more of the second terminals (INT-9) (FIG. 13A) and INT-11 (FIG. 13C). Any of the end groups (EG) disclosed herein can be suitable.

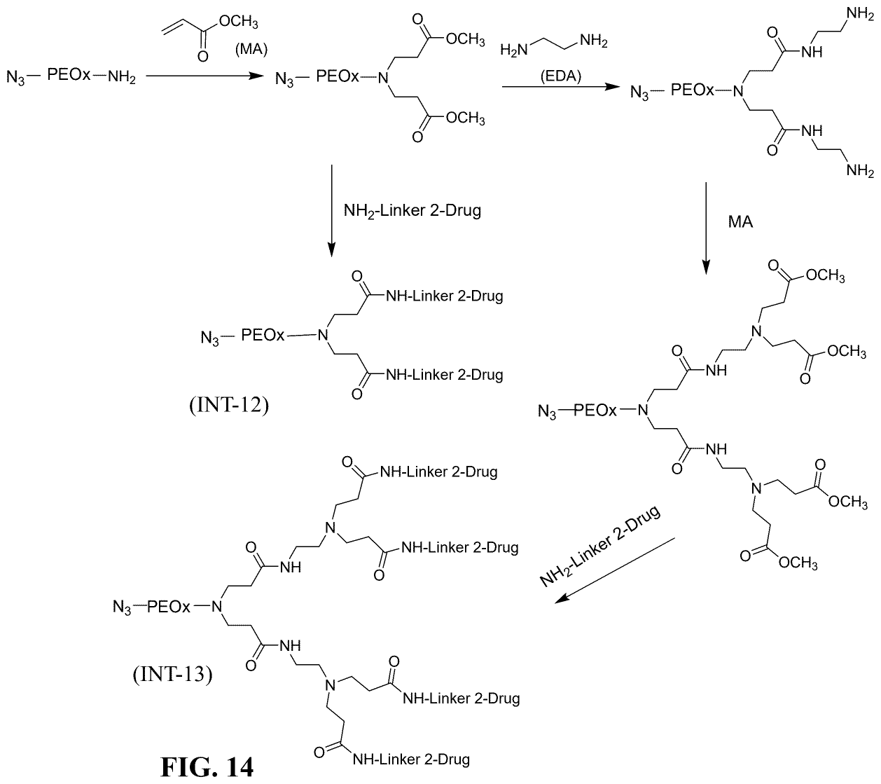

In some cases, a PEOX polymer can be functionalized and can have multiple PBAs, such as drug molecules, linked thereon at one or more of the second terminals of the PEOX polymer via one or more linkers L2 (FIG. 14). In some cases, a functionalized PEOX can comprise two PBAs, such as Exatecan, linked to one PEOX polymer at one or more of the second terminals via linkers L2 (INT-12) (FIG. 14). In some cases, a functionalized PEOX can comprise four PBAs, such as Exatecan, linked to one PEOX polymer at one or more of the second terminals via linkers L2 (INT-13) (FIG. 14). Any of the end groups (EG) disclosed herein can be suitable.

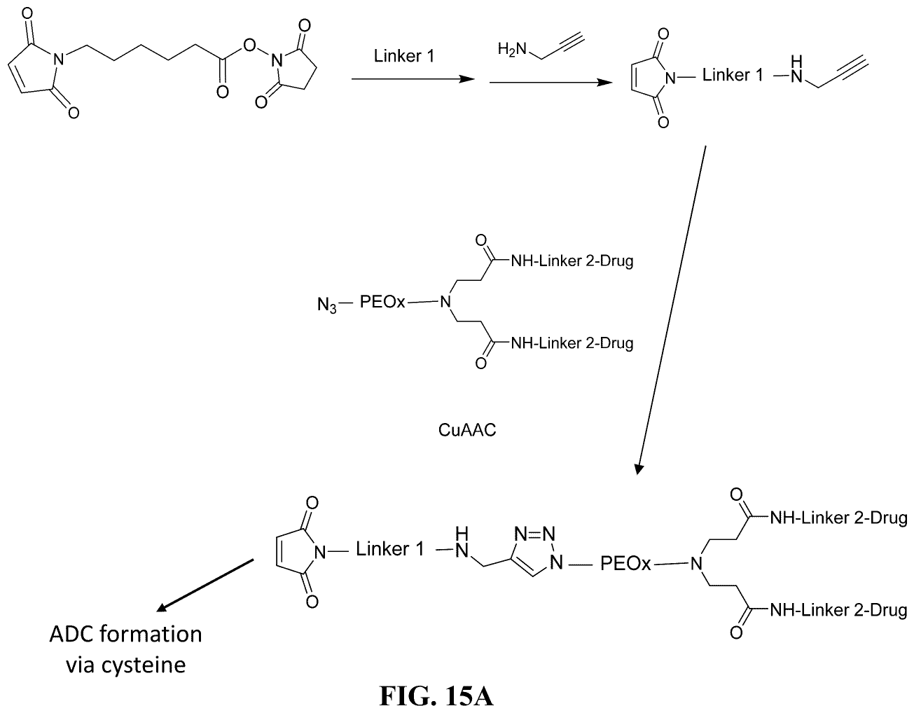

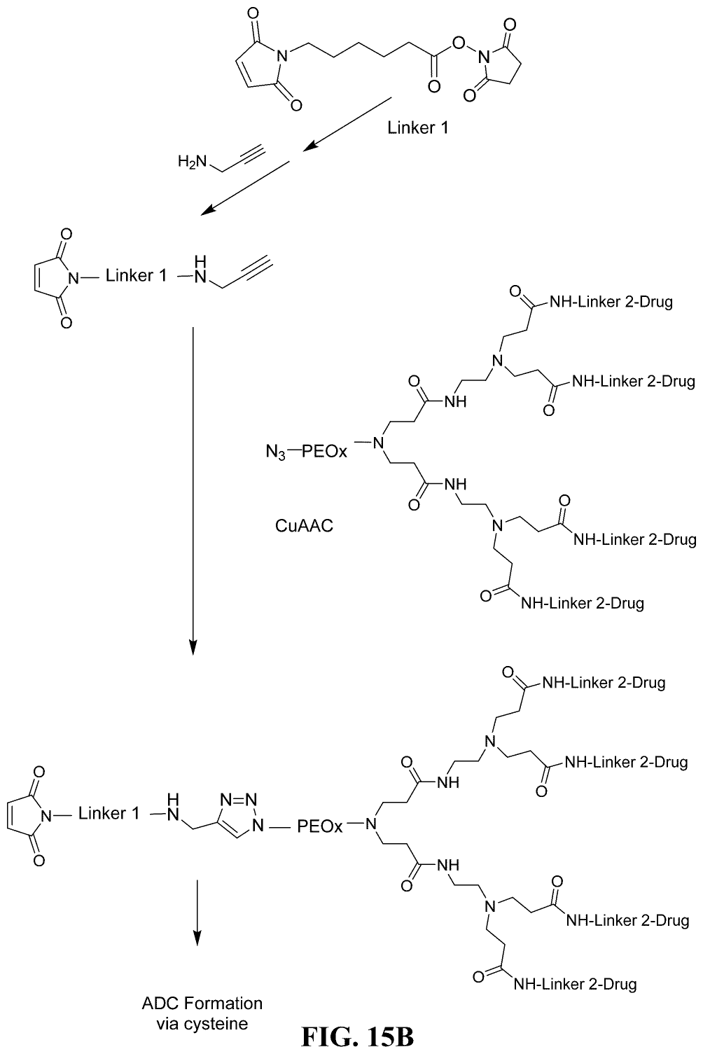

In some cases, a PEOX polymer can be linked to a linker L1 that can be reacted to link to a TBA, such as an antibody. Some non-limiting examples are schematically illustrated in FIG. 15A-FIG. 15B. In some cases, a functionalized PEOX can comprise a linker L1 and two PBAs, such as Exatecan, linked to one PEOX polymer at one or more of the second terminals via linkers L2 (FIG. 15A). In some cases, a functionalized PEOX can comprise a linker L1 and four PBAs, such as Exatecan, linked to one PEOX polymer at one or more of the second terminals via linkers L2 (FIG. 15B). The complexes shown in FIG. 15A-FIG. 15B can further react via cysteine residues of an antibody to form an ADC. With a proper reaction scheme disclosed herein, a functionalized PEOX comprising a linker L1, linkers L2 and two-four PBAs, can further react via lysine residues of an antibody to form an ADC (refer to at least FIG. 8, FIG. 10A, FIG. 11A, and the full disclosure herein).

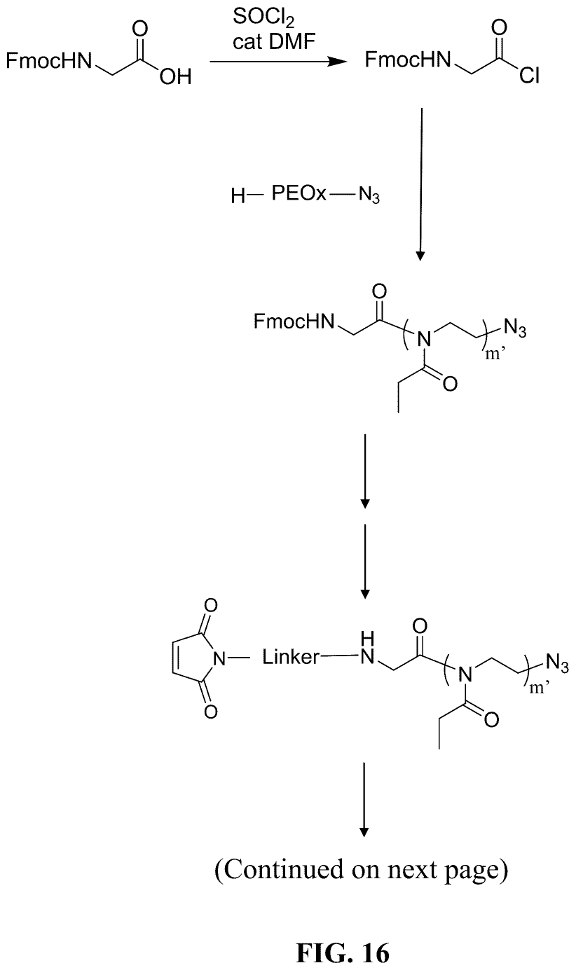

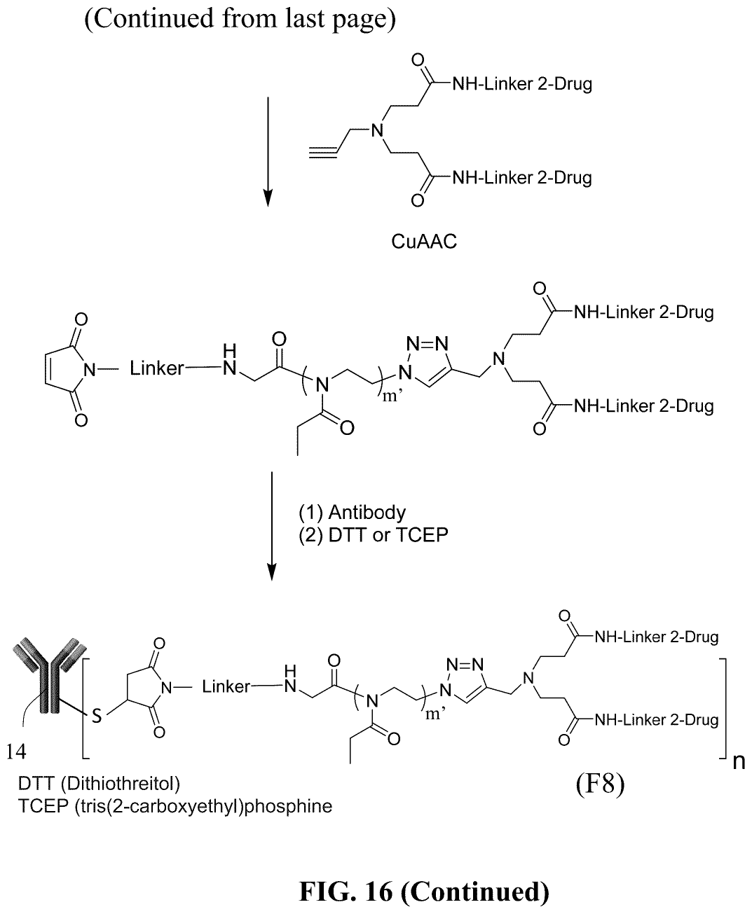

In some cases, a pharmaceutical composition can comprise an ADC having at least two PBAs covalently linked to each PEOX polymer. In some cases, a pharmaceutical composition can comprise an antibody (14) linked to a first terminal of a PEOX polymer via a linker L1, and at least two PBAs covalently linked to at least a second terminal of the PEOX polymer via one or more linkers L2. In some cases, a pharmaceutical composition can comprise an ADC having Formula F8 (FIG. 16). Representative examples are shown in FIG. 16 for the preparation of linker-PEOX-PBAs for the formation of an ADC comprising at least two PBAs covalently linked to each PEOX polymer, wherein at least a linker L1 is positioned between the TBA and the PEOX polymer and at least a linkers L2 is positioned between a second terminal of the PEOX polymer and a PBA. One or more linker-PEOX-PBAs can be attached to each antibody. The at least two PBAs can be the same or different. In some cases, a pharmaceutical composition can comprise an ADC having at least two different PBAs covalently linked to each PEOX polymer or each PEG polymer, wherein each of the at least two different PBAs selected from at least two different natural or synthetic small molecule-based drugs, inorganic-based drugs, toxin molecules having cytotoxicity, molecules having cytotoxicity to target cells, derivatives thereof, or a combination thereof.

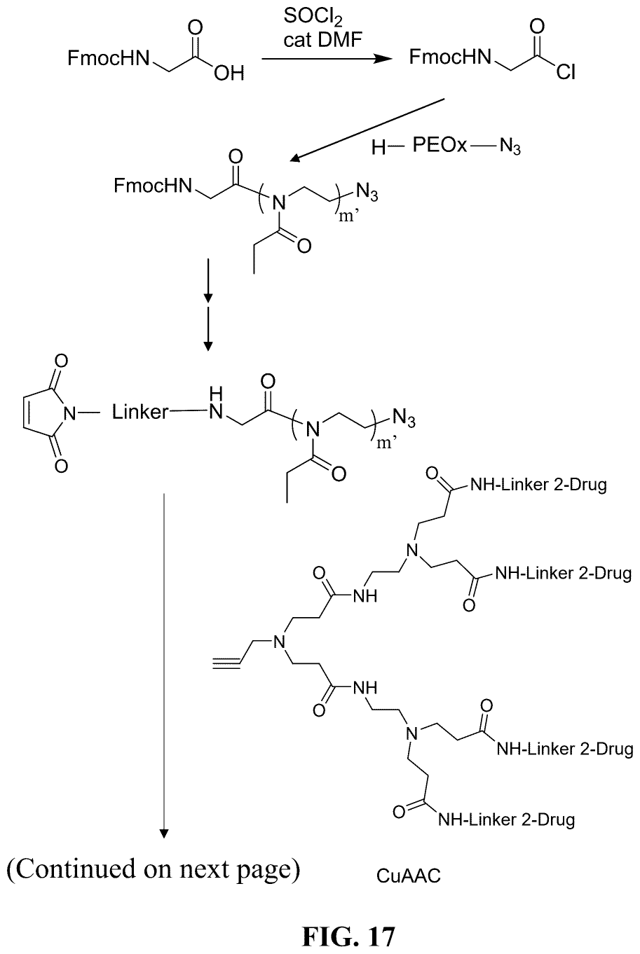

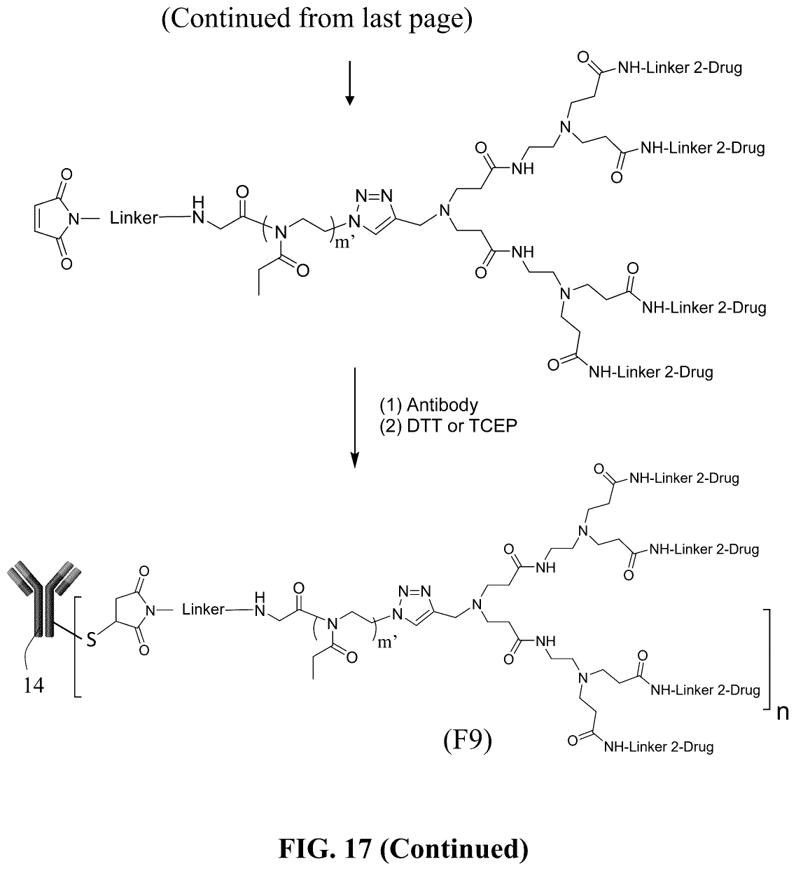

In some cases, a pharmaceutical composition can comprise an ADC having at least four PBAs covalently linked to each PEOX polymer. In some cases, the pharmaceutical composition can comprise an ADC having Formula F9 (FIG. 17). In some cases, a pharmaceutical composition can comprise an antibody (14) linked to a first terminal of a PEOX polymer via a linker L1, and at least four PBAs covalently linked to at least a second terminal of the PEOX polymer via one or more linkers L2. In some cases, a pharmaceutical composition can comprise an ADC having Formula F9 (FIG. 17). Representative examples are shown in FIG. 17 for the preparation of linker-PEOX-PBAs for the formation of an ADC comprising at least four PBAs covalently linked to each PEOX polymer, wherein at least a linker L1 is positioned between the TBA and the PEOX polymer and at least a linker L2 is positioned between a second terminal of the PEOX polymer and a PBA. One or more linker-PEOX-PBAs can be attached to each antibody.

The pharmaceutical composition of this disclosure, can comprise at least a payload bioactive agent (PBA) that is covalently linked to the targeting bioactive agent (TBA) indirectly via a cleavable linker that is cleavable in vivo or in a target cell, wherein the cleavable linker can be positioned between the TBA and the PEOX polymer, such as linker L1, positioned between the PEOX polymer and the PBA, such as linker L2, or a combination thereof. In some cases, a pharmaceutical composition can comprise a cleavable linker L1 positioned between the TBA and the PEOX polymer. In some cases, a pharmaceutical composition can comprise a cleavable linker L2 positioned between the PEOX polymer and the PBA. In some cases, a pharmaceutical composition can comprise at least a cleavable linker L1 positioned between the TBA and the PEOX polymer and at least a linker L2 positioned between the PEOX polymer and the PBA.

In some cases, the pharmaceutical composition can comprise a compound having Formula F10 (FIG. 18A), Formula F11 (FIG. 18B), Formula F12 (FIG. 18C), Formula F13 (FIG. 18D), Formula F14 (FIG. 18E) that each can comprise polyethelenglyco (PEG) with 1-100 monomer units (m=1-100), or a combination thereof. In some cases, the pharmaceutical composition can comprise a compound having Formula F13 (FIG. 18D) comprising polyethelenglyco (PEG) with 24 monomer units. In some cases, the pharmaceutical composition can comprise a compound having Formula F14 (FIG. 18E) comprising polyethelenglyco (PEG) with 8 monomer units.

In some cases, a pharmaceutical composition can comprise an antibody-drug conjugate (ADC), wherein the TBA can be an antibody (14), such as an anti-HER2 antibody. In some cases, an antibody-drug conjugate of this disclosure (herein “Nano-ADC”) can comprise an antibody, a polyethelenglyco (PEG) with 1-100 monomer units (m=1-100), a cleavable linker, such as a linker GGFG, a linker VAPAB, or a combination thereof, and a PBA. Each Nano-ADC can comprise in a range of from 1-100 of the PBA.

In some cases, the pharmaceutical composition can comprise a compound comprising a PEOX polymer. In some cases, the pharmaceutical composition can comprise a compound comprising a PEOX polymer having Formula F15 (FIG. 18F), Formula F16 (FIG. 18G), Formula F17 (FIG. 18H), Formula F18 (FIG. 18I) or Formula F19 (FIG. 18J).

Although specific —S— bond and —N— bonds are depicted in figures and descriptions, all aforementioned conjugations and or bonds can be suitable. In some case, the pharmaceutical composition can comprise a Nano-ADC conjugated via any of the aforementioned bonds or conjugates, such as S— bond, N— bond, glycol bond, modified glycol bond, GlcNAc (N-acetylglucosamine), substituted GlcNAc, GlcNAc-galactose, GlcNAc-mannose, GlcNAc-glucose, GlcNAc-glucuronic acid, GlcNAc-fucose, GlcNAc-N-acetylneuraminic acid, site specific bond, random bond, bioconjugations to other amino acid residues of the antibody, such as tyrosine (Tyr), tryptophan (Trp), and methionine (Met), conjugations via genetically engineered non-natural amino acids (NNAA), enzymatic conjugations, or a combination thereof.

Although the specific locations of linkers, cleavable linkers or specific peptide linkers are illustrated in figures and descriptions as examples, any of the linkers disclosed herein can be positioned at L1, L2, or a combination thereof. In some cases, the Nano-ADC can comprise a linker positioned between the antibody and the polymer. In some cases, the Nano-ADC can comprise a linker positioned between the polymer and the PBA. In some cases, the Nano-ADC can comprise a linker positioned between the antibody and the polymer and a linker positioned between the polymer and the PBA. The linkers can be the same or different. The term “Linker”, “Linker L1”, “L1 Linker”, “L1”, “Linker L2”, “L2 Linker”, “L2”, “cleavable Linker” or a gametic variation, regardless of whether capitalized or not, refers to a linker either in its un-reacted form or a reacted form, i.e., incorporated in an ADC.

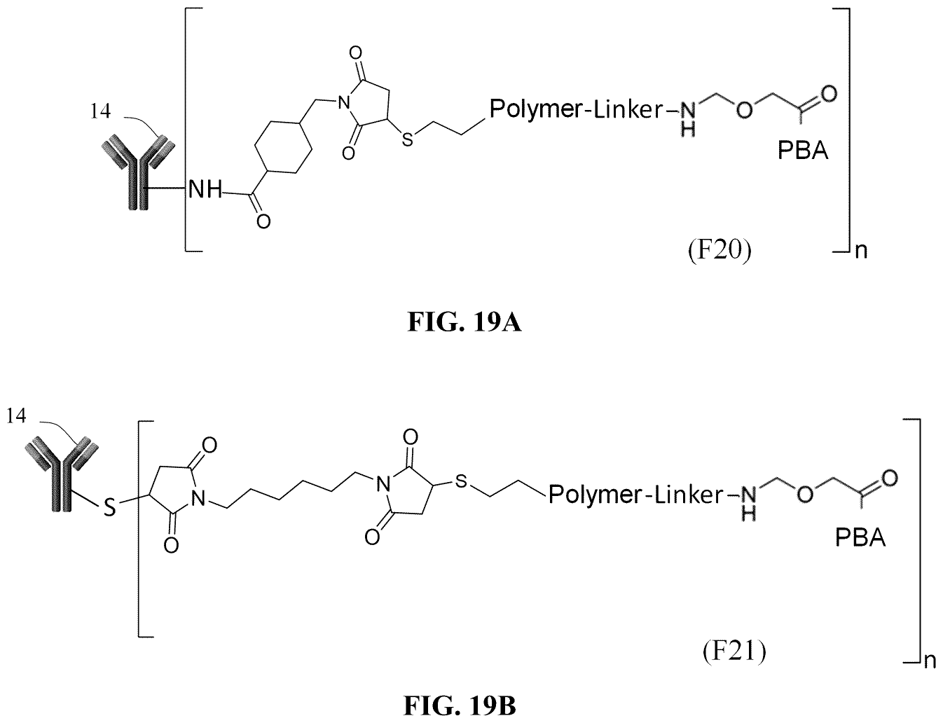

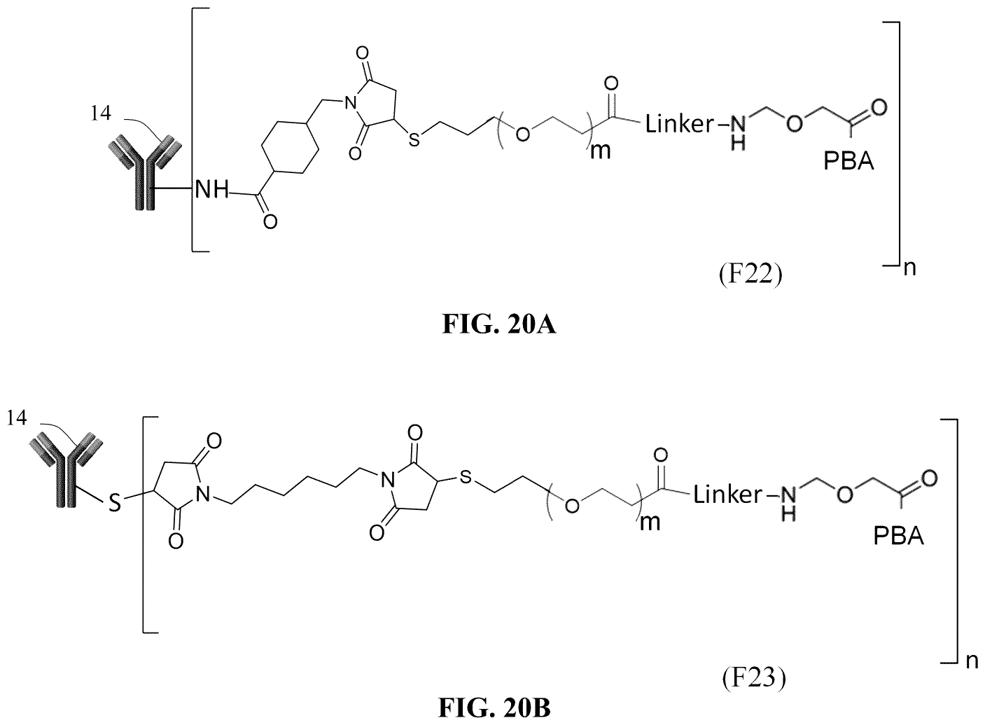

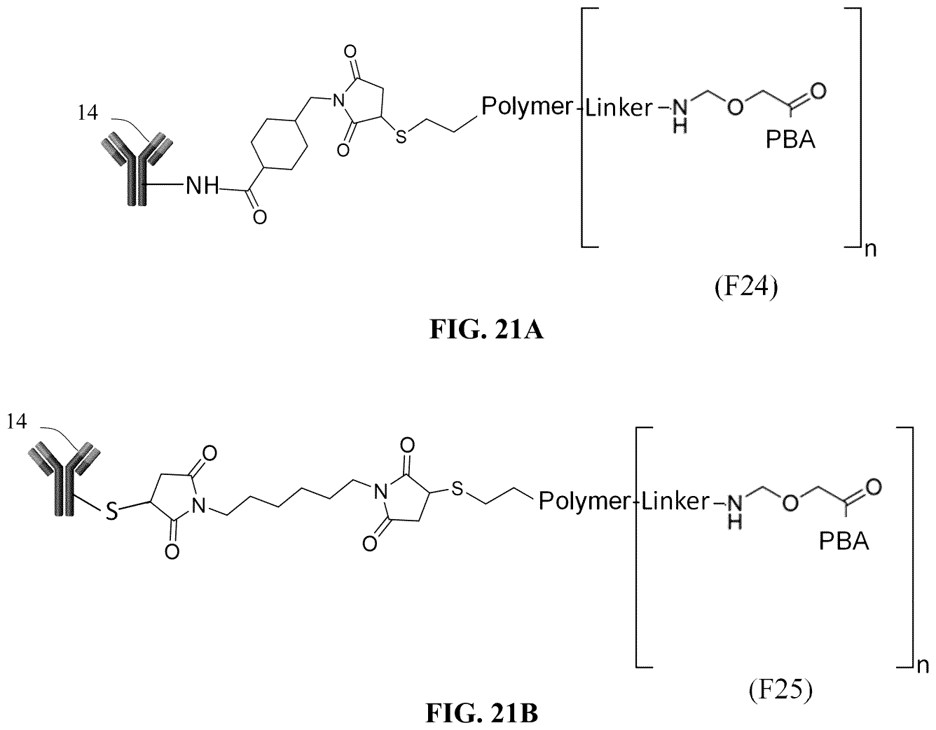

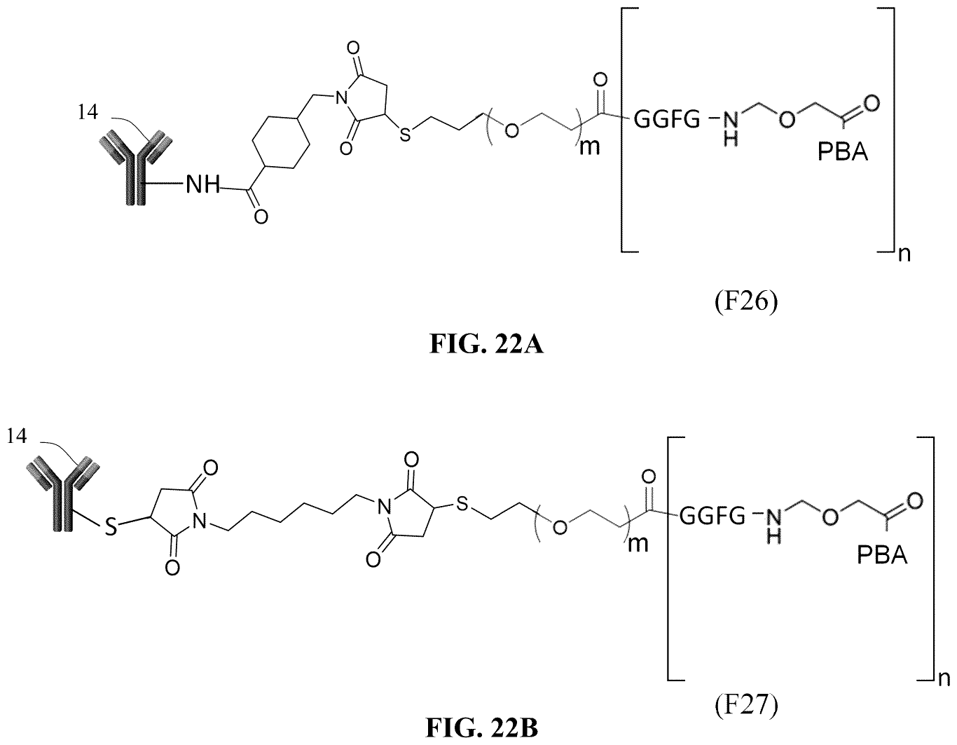

In some cases, a pharmaceutical composition can comprise an antibody-drug conjugate (ADC), wherein the TBA can be an antibody (14), such as an anti-HER2 antibody. In some cases, an antibody-drug conjugate (Nano-ADC) can comprise an antibody, a polymer, and at least a linker. In some cases, the pharmaceutical composition can comprise a compound having Formula F20-Formula F21 (FIG. 19A-FIG. 19B), Formula F22-Formula F23 (FIG. 20A-FIG. 20B), Formula F24-Formula F25 (FIG. 21A-FIG. 21B), Formula F26-Formula F27 (FIG. 22A-FIG. 22B), or a combination thereof. The linker can be a cleavable linker disclosed herein.

The PBA can be attached to the antibody via —S bond, —N bond, glycol bond, or a combination thereof. Some unlimiting examples are disclosed in FIG. 18A-FIG. 22B. Although specific cleavable linkers, such as the GGFG linker, are shown in unlimited examples in this disclosure, any cleavable linkers disclosed herein can be suitable.

In some cases, the pharmaceutical composition can comprise an antibody-drug conjugate (Nano-ADC) that can comprise an antibody, a polymer, a cleavable linker, such as a linker GGFG, a VAPAB, or a combination thereof, wherein, n can be an integer in a range of from 1-100. As mentioned above, the pharmaceutical composition can comprise a measured TBA:PBA in a range of from 1:0.01 to 1:100. In some cases, the pharmaceutical composition of this disclosure can comprise free antibody that is not conjugated with a payload bioactive agent (PBA).

The polymer can comprise polyethelenglyco (PEG), poly(2-oxazoline), poly(2-substituted oxazoline) that comprises poly(2-methyloxazoline), poly(2-ethyloxazoline), poly(2-propyloxazoline), poly(2-isopropyloxazoline) (PiPOX), poly(2-substituted oxazoline), or a combination thereof. The POX can comprise poly(2-methyloxazoline) (PMOX) in one example, poly(2-ethyloxazoline) (PEOX) in another example, poly(2-propyloxazoline) (PPOX) in yet another example, poly(2-isopropyloxazoline) (PiPOX) in yet another example, or a combination of two or more of the poly(2-substituted oxazoline)s in yet a further example, wherein the two or more of the poly(2-substituted oxazoline)s can be a repeating unit, also referred to as complex monomer, in the polyoxazoline polymer. The polyoxazoline (POX) is hydrophilic. The polyoxazoline (POX) can be free from monomers, either simple or complex monomers, having hydrophobic side chains, such as those having 4 or more carbons (C4 and above).

Some of examples of symmetrically or asymmetrically branched polymers (SBP) are described in detail in U.S. Pat. Nos. 8,591,904, 8,597,653, 10,688,048, and 9,944,755, hereby incorporated by reference.

The modified SBPs can be obtained, for example, through chemically linking functional groups on, for example, symmetrically branched PAMAM or PPI dendrimers, commercially available from Aldrich, polyether dendrimers, polyester dendrimers, comb-branched/star-branched polymers, such as, those containing PEO, PEG, PMOX or PEOX, polystyrene, and comb-branched dendrigrafts, such as, those containing PEOX, PMOX or PEI. The synthetic procedures for making such SBPs/dendrimers are known and described above and hereafter.

In some cases, the higher branching densities of SBP's can render the polymers molecularly compact with a well-defined interior void space, which makes such molecules suitable as a carrier for water insoluble or poorly water soluble drugs.

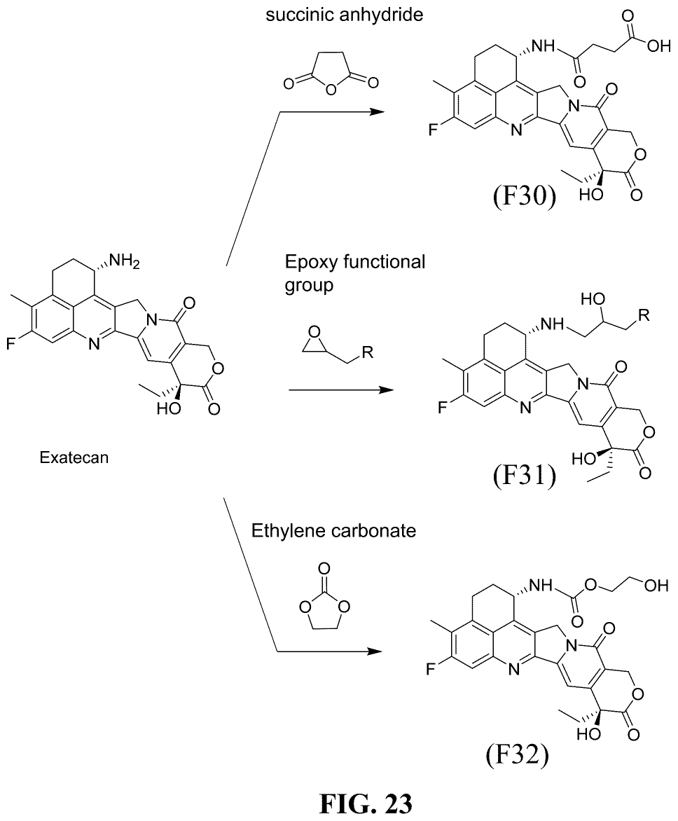

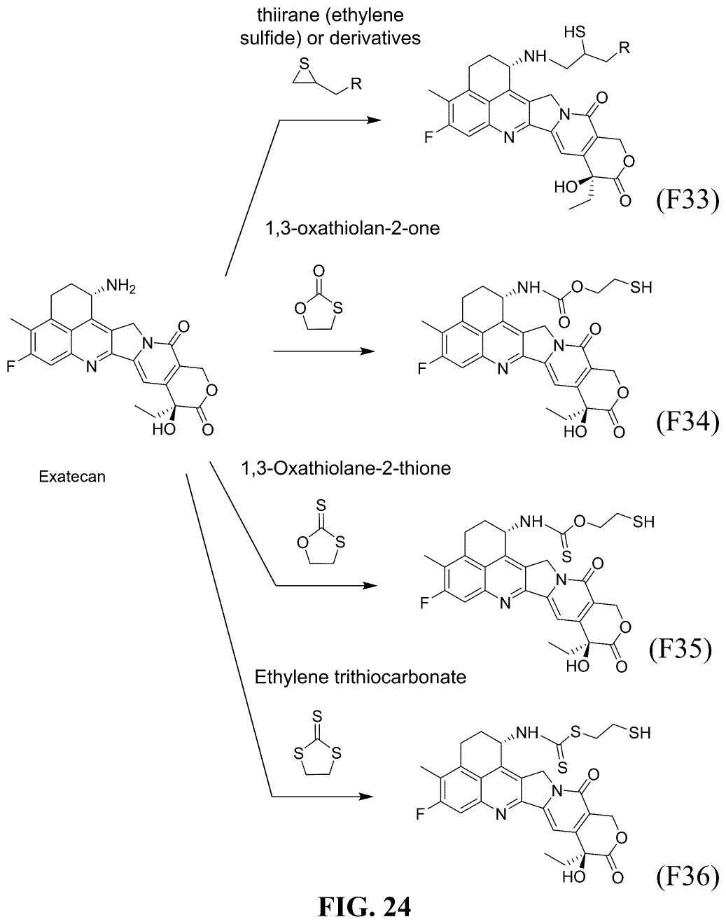

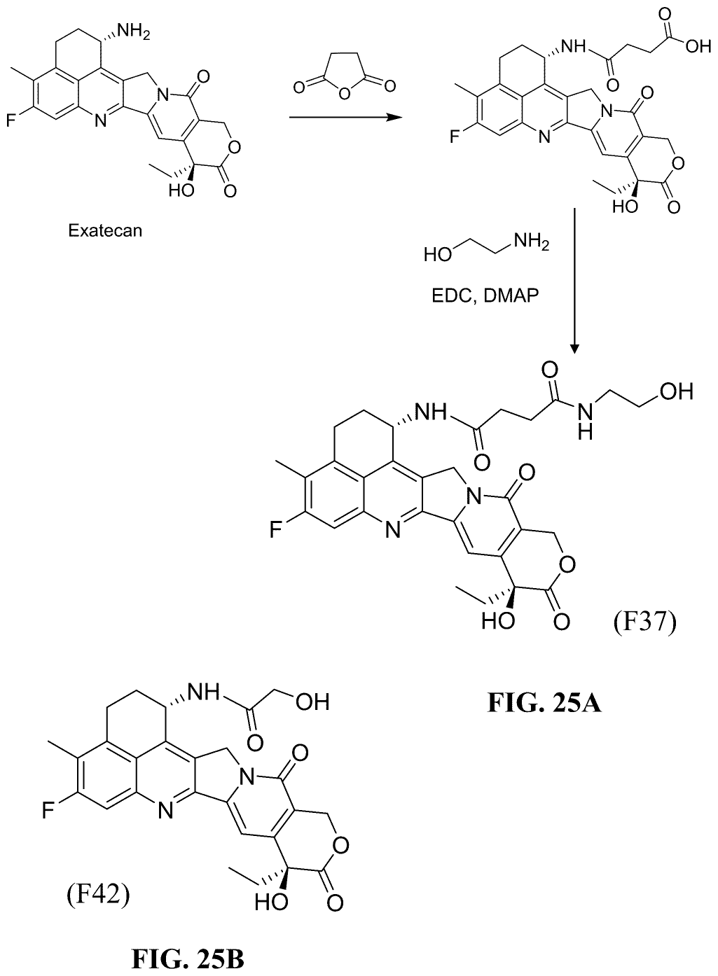

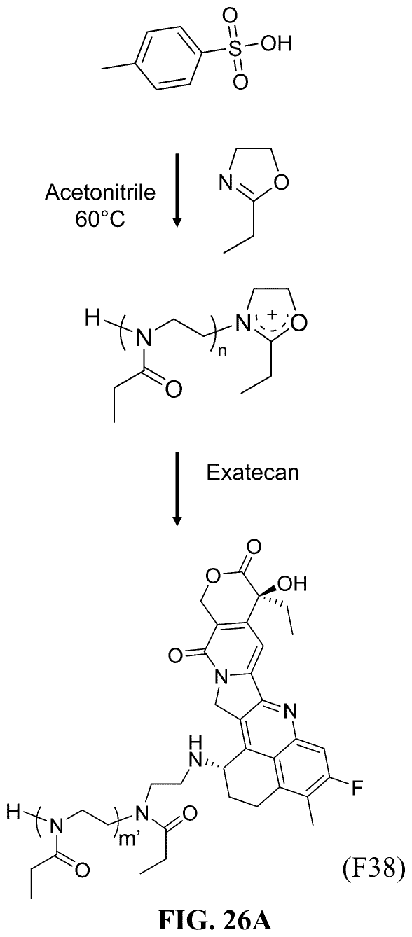

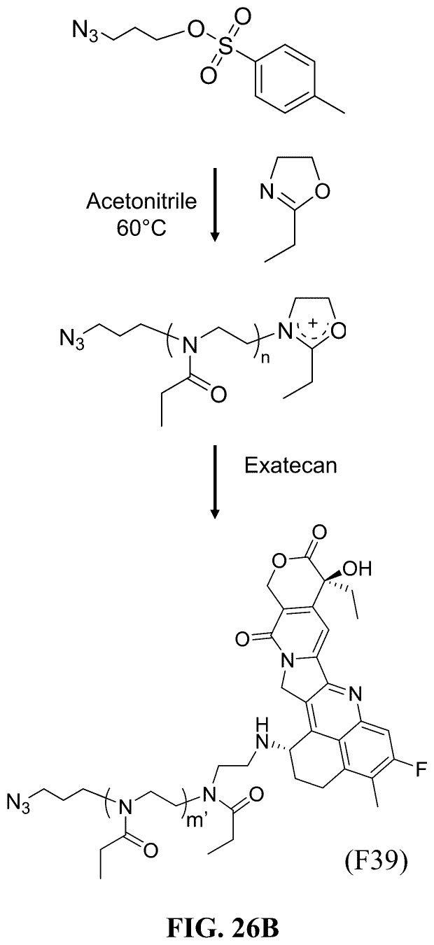

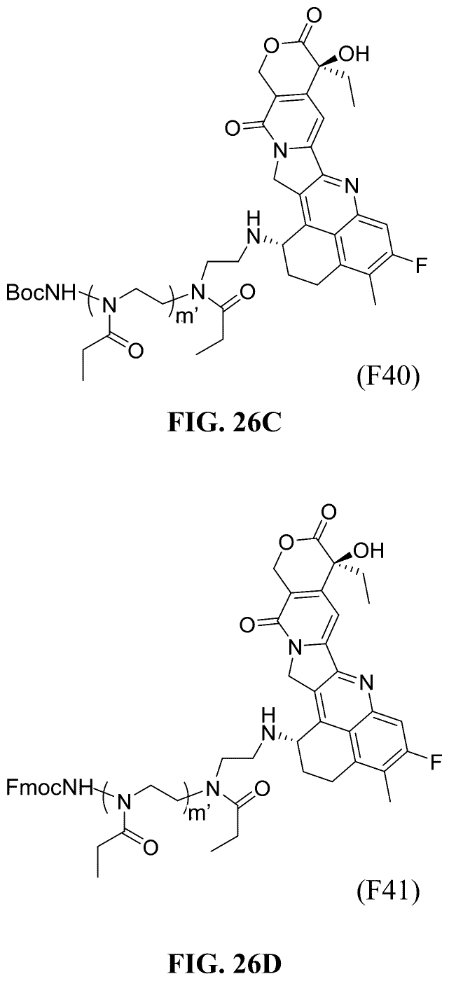

In some cases, a pharmaceutical composition can comprise payload bioactive agent (PBA) that can comprise modified exatecans (mExa) or exatecan derivatives. In some cases, a pharmaceutical composition can comprise at least a compound having a formula selected from Formula F30 to Formula F41 (FIG. 23, FIG. 24, FIG. 25A-FIG. 25B, FIG. 26A-FIG. 26D), as disclosed above and hereafter, and a combination thereof. For comparison, a modified exatecan known as deruxtecan (Dxd) is also shown (F42, FIG. 25B).

In some cases, a pharmaceutical composition can comprise a succinic anhydride modified exatecan having Formula F30 that can be produced via a reaction scheme disclosed in FIG. 23. In some cases, a pharmaceutical composition can comprise an epoxy modified exatecan having Formula F31 that can be produced via a reaction scheme disclosed in FIG. 23. The epoxy can comprise an R group that can be —OH or glycidol derivatives. In some cases, a pharmaceutical composition can comprise an ethylene carbonate modified exatecan having Formula F32 that can be produced via a reaction scheme disclosed in FIG. 23.

In some cases, a pharmaceutical composition can comprise a 2-thiirane methanol or a derivative modified exatecan having Formula F33 that can be produced via a reaction scheme disclosed in FIG. 24, wherein R group can comprise —OH or thiirane, such as ethylene sulfide derivatives. In some cases, a pharmaceutical composition can comprise a 1,3-oxathiolan-2-one modified exatecan having Formula F34 that can be produced via a reaction scheme disclosed in FIG. 24. In some cases, a pharmaceutical composition can comprise a 1,3-Oxathiolane-2-thione modified exatecan having Formula F35 that can be produced via a reaction scheme disclosed in FIG. 24. In some cases, a pharmaceutical composition can comprise an Ethylene trithiocarbonate modified exatecan having Formula F36 that can be produced via a reaction scheme disclosed in FIG. 24.

In some cases, a pharmaceutical composition can comprise a modified exatecan having Formula F37 that can be produced via a reaction scheme disclosed in FIG. 25A. In some cases, a pharmaceutical composition can comprise a modified exatecan having Formula F42 disclosed in FIG. 25B. Formula F42 is also known as deruxtecan.

In some cases, a pharmaceutical composition can comprise a modified exatecan having Formula F38 that can be produced via a reaction scheme disclosed in FIG. 26A. In some cases, a pharmaceutical composition can comprise a modified exatecan having Formula F39 that can be produced via a reaction scheme disclosed in FIG. 26B. In some cases, a pharmaceutical composition can comprise a modified exatecan having Formula F40 that can comprise a Boc functional group as disclosed in FIG. 26C. In some cases, a pharmaceutical composition can comprise a modified exatecan having Formula F41 that comprise a Fmoc functional group as disclosed in FIG. 26D.

In some cases, the enzymatically-cleavable peptide linker can comprise a GGFG linker, a linker having Gly-Gly-Phe-Gly short peptide that can comprise peptide bonds that can be cleaved by enzyme in vivo or in vitro, an aforementioned VAPAB linker, or a combination thereof.

In some cases, the linker L1 can comprise at least one of the cleavable linker comprising at least a cleavable bond. In some cases, the linker L1 can comprise at least one of the cleavable linker comprising at least a cleavable bond. In some cases, the linker L1 can comprise at least one of the cleavable polypeptide linker comprising at least a cleavable peptide bond. In some cases, the linker L1 can comprise at least a polypeptide linker GGFG. In some cases, the linker L2 can comprise at least one of the cleavable linker comprising at least a cleavable bond. In some cases, the linker L2 can comprise at least one of the cleavable polypeptide linker comprising at least a cleavable peptide bond. In some cases, the linker L2 can comprise at least a polypeptide linker GGFG.

In some cases, the targeting bioactive agent (TBA) can be selected from a peptide, a protein, DNA, RNA, nucleic acids, oligo nucleic acids, mRNA, siRNA, sgRNA, a monoclonal antibody, a fragment of a monoclonal antibody, a polyclonal antibody, a fragment of a polyclonal antibody, a synthetic antibody, a fragment of a synthetic antibody, antigen-binding portions thereof, a ligand of cell surface receptor, an agonist, an activator, an inhibitor, an antagonist, a naked nucleic acid, a nucleic acid, a ribozyme, a virus, a virus-like particles and a combination thereof, Alemtuzumab, alemtuzumab, an antisense nucleic acid, anti-CD19 antibodies, anti-CD20 antibodies, anti-CTLA-4 (cytotoxic T lymphocyte-associated antigen) antibodies, anti-LAG3 (lymphocyte activation gene-3) antibodies, anti-PD1 antibodies KEYTRUDA® (pembrolizumab), anti-PD-L1 antibodies, anti-TIM-3 (T cell immunoglobulin and mucin domain-3) antibodies, Atezolizumab, BAVENCIO® (avelumab), Bevacizumab, bevacizumab, Blinatumomab, Brentuximab, Cetuximab, cetuximab, chimeric antigen receptor or chimeric antigen T-cell receptor (CAR-T), cytokines, Daratumumab, Denosumab, Dinutuximab, Durvalumab, Elotuzumab, gemtuzumab, granulocyte colony stimulating factor (G-CSF), Ibritumomab, IMFINZI® (durvalumab), Inotuzumab, interferon α, interferon α2a, interleukins, Ipilimumab, lectins, Necitumumab, Neupogen (Filgrastim), Obinutuzumab, Ofatumumab, Olaratumab, OPDIVO® (nivolumab), Panitumumab, Pembrolizumab, Pertuzumab, Ramucirumab, Rituximab, rituximab, Siltuximab, Tislelizumab (BGB-A317), Toripalimab, siRNAs, T-cell receptor (TCR), TECENTRIQ® (atezolizumab), tositumomab, trastuzumab, and a combination thereof.

In some cases, the TBA can be selected from Alemtuzumab, Atezolizumab, Avelumab, Bevacizumab, Blinatumomab, Brentuximab, Cetuximab, Daratumumab, Denosumab, Dinutuximab, Durvalumab, Elotuzumab, GEMTUZUMAB, Ibritumomab, Inotuzumab, Ipilimumab, Necitumumab, Nivolumab, Obinutuzumab, Ofatumumab, Olaratumab, Panitumumab, Pembrolizumab, Pertuzumab, Ramucirumab, Rituximab, Siltuximab, Tislelizumab (BGB-A317), Toripalimab, Trastuzumab, and a combination thereof. In some cases, the TBA can be selected from one or more cancer treatment monoclonal antibodies.

In some cases, the TBA can be selected from ABT-700, ABT-806, Anti-AGS16 (Hu IgG2a), Anti-CD19, Anti-CD22, Anti-CD22 (Hz IgG1), Anti-CD79b (Hz IgG1), Anti-DLL3 (Rovalpituzumab), Anti-LIV1 (Hz IgG1), Antimesothelin (IgG1), Anti-PSMA (Hu IgG1), cAC10 (SGN-30, Ch-IgG1), CR-011 (Hu IgG2), DS6 (Hu IgG1), Enfortumab, G5/44 (Hz IgG4), Hp67.6 (Hz IgG4), hRS7 IgGk, huB4 (Hz IgG1), huN901 (Hz IgG1), IgG1, K7153A humanized IgG1, M9346A, Nbt062, Anti-CD138 (Ch IgG4), Tisotumab, Trastuzumab (Herceptin), Trastuzumab (Hz IgG1), and a combination thereof.

In some cases, the targeting bioactive agent can have binding affinity to DLL3, gpNMB, PSMA, CD22, ABT-700, LIV-1, FOLR1, CD56, CD19, CD138, Mesothelin, CA6, EGFR, CD37, ENPP3, CD33, CD30, HER2, CD79b, Nectin4, Trop-2, BCMA, Tissue factor, or a combination thereof.

In some cases, the payload bioactive agent (PBA) can comprise a natural or synthetic small molecule-based drug, inorganic-based drug, a toxin molecule having cytotoxicity, a molecule having cytotoxicity to the target cell, a derivative thereof, or a combination thereof. In some cases, the PBA can comprise 5-Fluorouracil (5-FU), alkylators, anti-metabolites, cabazitaxel, Calicheamicin, Calicheamicin derivatives, camptothecin, camptothecin derivatives, capecitabine, cell division inhibitors or microtubule inhibitors, ciprofloxaxin, cyclophosphamide, deruxtecan, deruxtecan derivatives, DNA cleavage agent, DNA crosslinking agent, DNA replicating inhibitor, docetaxel, dolastatin analogs, doxorubicin, duocarmycin analogs, etoposide, everolimus, exatecan, exatecan derivatives, topoisomerase inhibitors, gemcitabine, irinotecan (CPT-11), larotaxel, maytansinoids (DM1, DM4), milataxel, MMAE (monomethyl auristatin E), MMAF (monomethyl auristatin F), MMAH (monomethyl auristatin H), mTOR inhibitor, one or more ATP-competitive mTORC1/2 inhibitors, one or more dual PI3K-mTOR inhibitors, ortataxel, ozogamicin, paclitaxel, pemetrexed, pyrrolobenzodiazepine dimer (PBD), rapamycin, ridaforolimus, SN-38, taxane, temsirolimus, tesetaxel, Topoisomerase 1 (Top 1) inhibitor, Topoisomerase 2 (Top 2) inhibitor, topotecan, torin-1, torin-2, vinblastine, vinca alkaloids, vincristine, vinorelbine, vistusertib, zotarolimus, biological toxins, ricin, or a combination thereof.

The term “Drug” or “Exa” used in the formulas represents any PBAs disclosed herein and is not limited to a particular drug, or PBA. The term “Linker”, “Linker 1” or “Linker 2” represents any linkers disclosed herein and is used for illustration purposes only. The Linker, Linker 1 (also shown as L1) and Linker 2 (also shown as L2) can be the same or different or in a combination thereof.

In some cases, the pharmaceutical composition can comprise a mixture of TBA, wherein some of the TBA can be covalently linked to the PBA and some of the TBA can be non-covalently mixed with the PBA leading to more TBAs than the PBAs. In some cases, some antibody molecules can be free from bound drugs, i.e., free antibody without conjugated to a drug molecule. In some cases, the molar ratio of TBA:PBA can be 100:1, 20:1, 10:1, 5:1, 4;1, 3;1, or 2:1 (on a reverse ratio of 1:0.01, 1:0.1, 1:0.2, 1:0.25, 1:0.3, and 1:0.5). In some cases, the molar ratio of antibody to a drug can be 100:1, 10:1, 5:1, 4;1, 3;1, or 2:1 (on a reverse ratio of 1:0.01, 1:0.1, 1:0.2, 1:0.25, 1:0.3, and 1:0.5).

In some cases, the pharmaceutical composition disclosed herein can comprise a molar ratio of the TBA to the PBA in a range of 1:0.1 to 1:100, 1:1 to 1:100. In some cases, the pharmaceutical composition disclosed above can comprise a molar ratio of the TBA to the PBA, in a range of 1:0.1 to 1:100, 1:1 to 1:100, 1:2 to 1:100, 1:4 to 1:100, 1:8 to 1:100, 1:12 to 1:100, 1:20 to 1:100, 1:24 to 1:100, 1:48 to 1:100 and 1:64 to 1:100. In some cases, the pharmaceutical composition disclosed above can comprise a molar ratio of the TBA to the PBA, in a range of from 1:2 to 1:24. In some cases, the pharmaceutical composition disclosed above can comprise a molar ratio of the TBA to the PBA, in a range of from 1:4 to 1:24. In some cases, the pharmaceutical composition disclosed above can comprise a molar ratio of the TBA to the PBA, in a range of from 1:8 to 1:24. In some cases, the pharmaceutical composition disclosed above can comprise a molar ratio of the TBA to the PBA, in a range of from 1:16 to 1:24. One advantage of the ADC disclosed herein is that the number of the drug attached to each TBA can be more than two in one example, more than four in another example, more than 8 in yet another example, more than 10 in yet another example, more than 12 in yet another example, more than 16 in yet another example, and more than 24 in yet another example.

The polymer disclosed above and hereafter can be suitable and can comprise a linear polymer, a branched polymer, a symmetrically branched polymer, an asymmetrically branched polymer, a dendrimer, a dendrigraft polymer, a comb-branched polymer, a star-branched polymer, or a combination thereof. The polymer is water soluble. In examples, the polymer can be dissolved in water to produce a 12% weight percent or higher water solution.

In any of pharmaceutical compositions disclosed above and hereafter, the polymer can comprise a polyoxazoline (POX) that comprises a linear portion, a branched portion, or a combination thereof. The polymer can comprise a plurality of linear portions joined together in one example, one or more linear portions joined with one or more branched portions in another example, one or more branched portions joined together in yet another example. Each of the linear portions can be independently of various lengths, modifications, or a combination thereof. Each of the branched portions can be independently of various lengths, number of branches, modifications, or a combination thereof.

Suitable to the pharmaceutical composition, process, method and use disclosed herein throughout this disclosure, the term “bioactive agent” refers to a substance that can be a natural or synthetic small molecule-based drug, inorganic-based drug, biological drug, natural or synthetic large molecule-based drug, modifications and/or derivatives thereof, or a combination thereof, as disclosed herein. The bioactive agent can include a natural or synthetic small molecule-based drug, inorganic-based drug, biological drug, natural or synthetic large molecule-based drug, modifications and/or derivatives thereof, or a combination thereof, wherein at least one drug is poorly water soluble or water insoluble. A drug of interest can be a small molecule, a salt thereof in which the molecule is modified to be water insoluble or poorly water soluble or can be a biological molecule which is modified to be water insoluble or poorly water soluble, particularly when a drug has improved properties, such as improved bioavailability, less toxicity, better pharmacokinetics, or a combination thereof, in a water insoluble or poorly water soluble form. Suitable examples can include drugs which are poorly water soluble or water insoluble or can be modified to be water insoluble or poorly water soluble for an improved property. The bioactive agent can include growth agents; AIDS adjunct agents; alcohol abuse preparations, such as, agents for treating dependence or withdrawal; Alzheimer's Disease treatment agents; Amyotrophic Lateral Sclerosis treatment agents; analgesics; anesthetics; anticonvulsants; antidiabetic agents; antidotes; antifibrosis therapy agents; antihistamines; anti-infective agents, such as, antibiotics, antivirals, antifungals, amebicides, antihelmintics, antimalarials, leprostatics and so on; antineoplastic agents; antiparkinsonian agents; antirheumatic agents; appetite stimulants; biological response modifiers; biologicals; blood modifiers, such as, anticoagulants, colony stimulating factors, hemostatics, plasma extenders, thrombin inhibitors and so on; bone metabolism regulators; cardioprotective agents; cardiovascular agents, such as, adrenergic blockers, adrenergic stimulators, ACE inhibitors, antiarrhythmics, antilipemic agents, calcium channel blockers, diuretics, vasopressors and so on; CNS stimulants; cholinesterase inhibitors; contraceptives; fertility treatment agents; ovulation stimulators; cystic fibrosis managements agents; detoxifying agents; diagnostics; dietary supplements; dopamine receptor agonists; endometriosis management agents; enzymes; erectile dysfunction treatment agents; foot care products; gastrointestinal (GI) treatment agents, such as antacids, antidiarrheals, antiemetics, antiflatulants, bowel evacuants, digestive enzymes, histamine receptor agonists, laxatives, proton pump inhibitors, prostaglandins and so on; Gaucher's Disease treatment agents; gout treatment agents; homeopathic remedies; skin treatments; vitamins; nutrients; hormones; hypercalcemia management treatment agents; hypocalcemia management treatment agents; immunomodulators; immunosuppressants; levocarnitine deficiency treatment agents; mast cell stabilizers; migraine treatment agents, motion sickness treatment products, such as, Benadryl and Phenergan; decongestants; antihistamines; cough suppressants; multiple sclerosis treatment agents; muscle relaxants; nasal treatment agents, such as, anti-inflammatories; smoking cessation aids; appetite suppressants; nucleoside analogs; obesity management agents; ophthalmic preparations, such as, antibiotics, antiglaucoma agents, artificial tears, lubricants and so on; sexual aids; osteoporosis treatment agents; otic preparations, such as, antiinfectives and cerumenolytics; minerals; oxytocics; parasympatholytics; parasympathomimetics; patent ductus arteriosus agents; phosphate binders; porphyria agents; prostaglandins; psychotherapeutic agents; radiopaque agents; respiratory agents, such as, antiinflammatories, antitussives, bronchodilators, decongestants, expectorants, leukotrienes antagonists, surfactants and so on; salt substitutes; sedatives; hypnotics; skin and mucous membrane treatment agents, such as, acne treatments; anorectal treatment agents, such as, hemorrhoid treatments and enemas; antiperspirants; antipruritics; antipsoriatic agents; antiseborrheic agents; burn treatment agents; cleansing agents; depigmenting agents; emollients; hair growth retardants; hair growth stimulators; keratolytics; hair problem treatment agents; mouth and throat problem treatment agents; photosensitizing agents; wart treatment agent; wound care treatment agents, or a combination thereof. The bioactive agent can also include over the counter pharmaceutics and products, such as, deodorants; Tourette's Syndrome agents; tremor treatments; urinary tract agents, such as, acidifiers, alkalinizers; antispasmodics; benign prostatic hyperplasia treatment agents; calcium oxalate stone preventors; enuresis management agents; vaginal preparations, such as, antiinfectives, hormones and so on; vasodilators; vertigo treatment agents, Wilson's Disease treatments and so on.

Further examples of bioactive agent can include forms of drugs which may be modified, for example, as salts, ionized or hydrophilic forms that can be modified to remove such functional groups, modifications and the like to yield non-modified or other forms of bioactive agents which are poorly water soluble or water insoluble. If two or more bioactive agents are comprised in the pharmaceutical composition, at least one of the bioactive agents can be or has been modified to be water insoluble or poorly water soluble. Examples of such bioactive agents can include, analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate and the like); anesthetics (e.g., cyclopropane, enflurane, halothane, isoflurane, methoxyflurane, nitrous oxide, propofol and the like); antiasthmatics (e.g., azelastine, ketotifen, traxanox, amlexanox, cromolyn, ibudilast, montelukast, nedocromil, oxatomide, pranlukast, seratrodast, suplatast tosylate, tiaramide, zafirlukast, zileuton, beclomethasone, budesonide, dexamethasone, flunisolide, triamcinolone acetonide and the like); antibiotics (e.g., neomycin, streptomycin, chloramphenicol, cephalosporin, ampicillin, penicillin, tetracycline and the like); quinolone, fluoroquinolone, antidepressants (e.g., nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, trimipramine maleate, protriptyline hydrochloride and the like); antidiabetics (e.g., biguanides, hormones, sulfonylurea derivatives, and the like); antifungal agents (e.g., griseofulvin, ketoconazole, amphotericin B, nystatin, candicidin and the like); antihypertensive agents (e.g., propanolol, propafenone, oxyprenolol, nifedipine, reserpine, trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine and the like); anti-inflammatories (e.g., non-steroidal compounds, such as, indomethacin, naproxen, ibuprofen, ramifenazone, piroxicam and so on, and steroidal compounds, such as, cortisone, dexamethasone, fluazacort, hydrocortisone, prednisolone, prednisone and the like); antineoplastics (e.g., adriamycin, cyclophosphamide, actinomycin, bleomycin, daunorubicin, doxorubicin, epirubicin, gemcitabine, mitomycin, methotrexate, fluorouracil, carboplatin, carmustine (BCNU), methyl-CCNU, cisplatin, etoposide, interferons, camptothecin and derivatives thereof, phenesterine, Taxol and derivatives thereof, taxotere and derivatives thereof, vinblastine, vincristine, tamoxifen, etoposide, piposulfan and the like); antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, dantrolene and the like); immunosuppressive agents (e.g., cyclosporine, azathioprine, mizoribine, FK506 (tacrolimus), rapamycin (sirolimus) and the like); antimigraine agents (e.g., ergotamine tartrate, propanolol hydrochloride, isometheptene mucate, dichloralphenazone and the like); sedatives/hypnotics (e.g., barbiturates (e.g., pentobarbital, pentobarbital sodium, secobarbital sodium and the like) or benzodiazapines (e.g., flurazepam hydrochloride, triazolam, tomazeparm, midazolam hydrochloride and the like); antianginal agents (e.g., beta-adrenergic blockers, calcium channel blockers (e.g., nifedipine, diltiazem hydrochloride and the like) and nitrates (e.g., nitroglycerin, isosorbide dinitrate, pentaerythritol tetranitrate, erythrityl tetranitrate and the like)); antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine and the like); antimanic agents (e.g., lithium carbonate); antiarrhythmics (e.g., bretylium tosylate, esmolol hydrochloride, verapamil hydrochloride, amiodarone, encamide hydrochloride, digoxin, digitoxin, mexiletine hydrochloride, disopyramide phosphate, procainamide hydrochloride, quinidine sulfate, quinidine gluconate, quinidine polygalacturonate, flecamide acetate, tocamide hydrochloride, lidocaine hydrochloride and the like); antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium and the like); antigout agents (e.g., colchicine, allopurinol and the like); anticoagulants (e.g., heparin, heparin sodium, warfarin sodium and the like); thrombolytic agents (e.g., urokinase, streptokinase, altoplase and the like); antifibrinolytic agents (e.g., aminocaproic acid); hemorheologic agents (e.g., pentoxifylline); antiplatelet agents (e.g., aspirin, empirin, ascriptin and the like); anticonvulsants (e.g., valproic acid, divalproate sodium, phenyloin, phenyloin sodium, clonazepam, primidone, phenobarbitol, phenobarbitol sodium, carbamazepine, amobarbital sodium, methsuximide, metharbital, mephobarbital, mephenyloin, phensuximide, paramethadione, ethotoin, phenacemide, secobarbitol sodium, clorazepate dipotassium, trimethadione and the like); antiparkinson agents (e.g., ethosuximide and the like); antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like); agents useful for calcium regulation (e.g., calcitonin, parathyroid hormone and the like); antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate and the like); antiviral agents (e.g., interferon β, zidovudine, amantadine hydrochloride, ribavirin, acyclovir and the like); antimicrobials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium and the like), penicillins (e.g., ampicillin, amoxicillin, penicillin G benzathine, cyclacillin, ampicillin sodium, penicillin G K, penicillin V K, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, nafcillin sodium and the like), erythromycins (e.g., erythromycin ethylsuccinate, erythromycin, erythromycin estolate, erythromycin lactobionate, erythromycin stearate, erythromycin ethylsuccinate and the like), tetracyclines (e.g., tetracycline hydrochloride, doxycycline hyclate, minocycline hydrochloride and the like), and the like); anti-infectives (e.g., GM-CSF); bronchodilators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate); anticholinergic agents (e.g., ipratropium bromide); xanthines (e.g., aminophylline, dyphylline, metaproterenol sulfate, aminophylline); mast cell stabilizers (e.g., cromolyn sodium); inhalant corticosteroids (e.g., flunisolide, beclomethasone dipropionate monohydrate and the like), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide and the like); hormones (e.g., androgens (e.g., danazol, testosterone cypionate, fluoxymesterone, ethyltostosterone, testosterone enanthate, methyltestosterone, fluoxymesterone, testosterone cypionate and the like); estrogens (e.g., estradiol, estropipate, conjugated estrogens and the like), progestins (e.g., methoxyprogesterone acetate, norethindrone acetate and the like), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate and the like), thyroid hormones (e.g., levothyroxine sodium); and the like); and the like; hypoglycemic agents (e.g., human insulin, purified beef insulin, purified pork insulin, glyburide, chlorpropamide, glipizide, tolbutamide, tolazamide and the like); hypolipidemic agents (e.g., clofibrate, dextrothyroxine sodium, probucol, lovastatin, niacin and the like); proteins (e.g., DNase, alginase, superoxide dismutase, lipase and the like); nucleic acids (e.g., sense or anti-sense nucleic acids encoding any therapeutically useful protein, including any of the proteins described herein and the like); agents useful for erythropoiesis (e.g., erythropoietin); antiulcer or antireflux agents (e.g., famotidine, cimetidine, ranitidine hydrochloride and the like); antinauseants or antiemetics (e.g., meclizine hydrochloride, nabilone, prochlorperazine, dimenhydrinate, promethazine hydrochloride, thiethylperazine, scopolamine and the like); oil-soluble vitamins (e.g., vitamins A, D, E, K and the like); mitotane, visadine, halonitrosoureas, anthrocyclines, ellipticine and the like; STING inhibitors such as C-176, C-170 and C-171; STING activators, such as 3′,3′-cGAMP (3′,3′-cyclic GMP-AMP, Cyclic GMP-AMP, cGAMP); STING agonists such as SR-717 lithium, Alpha-mangostin or diABZI STING agonist (diABZI STING agonist-1, Compound 3), STING agonist-1 (G10), CF501, CF502, CF504, CF505, CF508, CF509, CF510, CF511 (Liu, et al., Cell Research, 1-19, 2022. https://doi.org/10.1038/s41422-022-00612-2), or MSA-2; STING antagonist such as SN-011 (GUN35901) or H-151; IDO inhibitors or IDO1 inhibitors such as Epacadostat (INCB24360), BMS-986205, PF-0684003, Navoximod, Indoximod, NLG802 (Indoximod prodrug) or LY3381916; and a combination thereof.

In some cases, the bioactive agent can comprise any one of the bioactive agents listed above and hereafter. In some cases, the bioactive agent can comprise two or more of the bioactive agents listed above and hereafter.

In some cases, in any of the pharmaceutical composition disclosed herein, the bioactive agent can comprise a natural or synthetic small molecule-based drug, inorganic-based drug, biological drug, natural or synthetic large molecule-based drug, a derivative thereof, or a combination thereof.

In some cases, the bioactive agent can comprise 7-ethyl-10-hydroxycamptothecin (SN-38). In some cases, bioactive agent can comprise 7-ethyl-10-hydroxycamptothecin (SN-38), irinotecan (also known as Camptosar, Campto, Onivyde, CPT-11), camptothecin (CPT), topotecan, or a combination thereof.

In some cases, the bioactive agent can comprise at least one STING polypeptide or a part thereof, a nucleic acid encoding the STING polypeptide or a part thereof, a STING inhibitor, a STING activator, a STING agonist, a STING antagonist, a STING modulating molecule, an IDO inhibitor, an IDO1 inhibitor, or a combination thereof. The bioactive agent can comprise RNA, mRNA, siRNA, sgRNA, DNA, oligo, or a combination thereof, that each encodes one of the aforementioned STING polypeptide or a part thereof, a STING inhibitor, a STING activator, a STING agonist, a STING antagonist, a STING modulating molecule, or the IDO inhibitor or IDO1 inhibitor.

In some cases, the bioactive agent can comprise STING (stimulator of interferon genes) protein, STING agonists, STING activators, STING inhibitors, STING antagonists or a combination thereof. In some cases, the bioactive agent can comprise one or more IDO or IDO1 inhibitors. Any of the STING protein, STING agonists, STING activators, STING inhibitors, STING antagonists, IDO inhibitors or IDO1 inhibitors, disclosed herein or discovered thereafter can be suitable. In some cases, the bioactive agent can comprise STING modulating molecules, such as benzimidazole compounds disclosed by Liu, et al. (Cell Research, 1-19, 2022), in patent publications WO2017175156A1 and WO2020156363, pyridinyl imidazole compounds disclosed in patent publications WO2019134705, WO2020010451 and US20200031825, or a combination.

The term “cancer” or “cancers” used herein and throughout this disclosure refers to cancer or tumor and can include malignant tumors and benign tumors such as solid tumors and cancers of the blood, such as leukemias. Malignant tumors can spread into, or invade, nearby tissues. In addition, as these tumors grow, some cancer cells can break off and travel to distant places in the body through the blood or the lymph system and form new tumors (metastatic tumor) far from the original tumor (primary cancer). A cancer can include a primary cancer or a metastatic tumor. The pharmaceutical composition disclosed herein can be a cancer treatment drug for treating one or more cancers. In some cases, the term “cancer” or “cancers” used herein can include one or more cancer selected from acoustic neuroma, Acute Lymphoblastic Leukemia (adult), Acute Lymphoblastic Leukemia (pediatric), Acute Myeloid Leukemia, adenocarcinoma, Anal Cancer, Anemia and Neutropenia (Low Red and White Blood Cell Counts), basal cell carcinoma, Basal Cell Skin Cancer, B-Cell Lymphomas (Diffuse Large B-Cell Lymphoma), B-Cell Lymphomas (Follicular Lymphoma), B-Cell Lymphomas (Mantle Cell Lymphoma), bile duct carcinoma, biliary track cancer, Bladder Cancer, bladder carcinoma, Bone Cancer, Brain Cancer (Gliomas), brainstem glioma, breast cancer, Breast Cancer (DCIS Breast Cancer), Breast Cancer (Invasive Breast Cancer), Breast Cancer (Metastatic Breast Cancer), triple-negative breast cancer, ER(+) locally advanced or metastatic breast cancer, bronchogenic carcinoma, Central Nervous System Cancers (Primary Central Nervous System Lymphoma), cervical cancer, choriocarcinoma, Chronic Lymphocytic Leukemia, chronic lymphocytic lymphoma, Chronic Myeloid Leukemia, colon cancer, colon carcinoma, Colorectal Cancer (CRC), diffuse large B cell lymphoma, ependymoma, Esophageal Cancer, Gallbladder and Bile Duct Cancers, gastric cancers, germinoma, glioblastoma astrocytoma, mixed gliomas, Graft-Versus-Host Disease, head and neck cancers, Head and Neck Cancers (Nasopharyngeal Cancer), Head and Neck Cancers (Oral Cancers), Head and Neck Cancers (Oropharyngeal Cancer), hemangioblastoma, hepatocellular carcinoma, hepatoma, Hodgkin Lymphoma, Kidney Cancer, leukemias, Liver Cancer, Lung Cancer, Lung Cancer (Non-Small Cell Lung Cancer—Early and Locally Advanced), Lung Cancer (Non-Small Cell Lung Cancer—Metastatic), Lung Cancer NSCLC (Non-Small Cell Lung Cancer), Lung Cancer (Small Cell Lung Cancer), lymphoid malignancy, Malignant Pleural Mesothelioma, medullary carcinoma, medullary thyroid carcinoma, medulloblastoma, melanoma, menangioma, Multiple Myeloma, Mycosis Fungoides/Sezary Syndrome, Myelodysplastic Syndromes, Myeloproliferative Neoplasms, neuroblastoma, neuroendocine tumor (advanced), Neuroendocrine Tumors, oligodendroglioma, ovarian cancer, pancreatic cancer, papillary adenocarcinomas, papillary carcinoma, papillary thyroid carcinoma, Peripheral T-Cell Lymphoma, perivascular epithelioid cell tumor (PEComa), perivascular epithelioid cell tumor (advanced unresectable or metastatic malignant), pheochromocytomas, pinealoma, Primary Cutaneous Lymphomas, prostate cancer, Prostate Cancer (Advanced Stage), Prostate Cancer (Early Stage), Rectal Cancer, recurrent endometrial cancer, relapsed ER(+) high-grade ovarian cancer, relapsed or refractory non-Hodgkin lymphoma, renal cancer (metastatic clear cell), renal cell carcinoma, retinoblastoma and brain metastases, Schwannoma craniopharyogioma, sebaceous gland carcinoma, seminoma, soft tissue sarcoma, squamous cell carcinoma, Squamous Cell Skin Cancer, Stomach Cancer, sweat gland carcinoma, Systemic Mastocytosis, testicular tumor, thyroid cancer, Uterine Cancer, uterine sarcoma, Waldenström Macroglobulinemia, and a combination thereof.

The term “a combination thereof” used for a combination of the bioactive agents disclosed above means a combination of two or more bioactive agents, wherein such combination does not have undesired effect, such as an undesired interaction between or among the bioactive agents. It is understood that some of combinations of the bioactive agents may not be suitable, or may not be desirable, such as those having undesired interactions. For example, a combination of theophylline and ciprofloxacin or warfarin and diflunisal may not be suitable. These combinations or any combinations determined by appropriate guidelines or regulations as not suitable are thus excluded.

In any of the pharmaceutical compositions disclosed above and hereafter, the nanoaggregate can have a weight ratio of polymer to bioactive agent in a range of from about 1:500 to about 500:1. The nanoaggregate can have a weight ratio of the polymer to the bioactive agent in a range of from 1:500 to about 500:1, 1:200 to 200:1, 1:100 to 200:1, 1:10 to 200:1, 1:5 to 200:1, 1:1 to 200:1, about 2:1 to about 200:1 in one example, about 2:1 to about 150:1 in another example, about 2:1 to about 120:1 in yet another example, about 2:1 to about 100:1 in yet another example, about 2:1 to about 80:1 in yet another example, about 2:1 to about 60:1 in yet another example, about 2:1 to about 40:1 in yet another example, about 2:1 to about 30:1 in yet another example, about 2:1 to about 20:1 in yet another example, about 2:1 to about 15:1 in another example, about 2:1 to about 10:1 in yet another example, about 2:1 to about 8:1 in yet another example, about 5:1 to about 10:1 in yet another example, about 5:1 to about 8:1 in yet another example, 5:1 in yet a further example, about 6:1 to about 8:1 in yet another example, 6:1 in yet a further example, 7:1 in yet a further example, 7.5:1 in yet a further example, and 8:1 in yet a further example. When pharmaceutical composition comprises two or more bioactive agents, the ratio of polymer to bioactive agent can be based on the total weight of polymer and the bioactive agents.

The pharmaceutical composition disclosed herein can be formulated for parenteral, oral, nasal, transdermal (topical), transmucosal, rectal administration, or a combination thereof and can comprise one or more pharmaceutical suitable carriers. In some cases, the pharmaceutical composition disclosed herein can be formulated for intravenous (IV), intradermal (ID), subcutaneous (SC), oral, transdermal (topical), transmucosal, rectal administration, or a combination thereof. In some cases, the pharmaceutical composition disclosed herein can be formulated for intravenous (IV), intradermal (ID), subcutaneous (SC), transdermal (topical) or transmucosal administration. In some cases, the pharmaceutical composition disclosed herein can be formulated for oral administration, such as tablets, capsules, oral spray, solutions, or suspensions. In some cases, the pharmaceutical composition disclosed herein can be formulated for nasal administration, such as nasal spray. The pharmaceutical suitable carriers disclosed herein can be suitable.

In some cases, the composition of this disclosure can comprise one or more of the compounds selected from Formula F30 to Formula F42. In some cases, the composition of this disclosure can comprise two or more of the compounds selected from Formula F30 to Formula F42.

In some cases, this disclosure can be directed an antibody-drug conjugate (ADC) (also referred to as “Nano-ADC”) comprising:
a targeting bioactive agent (TBA) comprising an antibody or a part thereof having binding affinity to a target molecule that comprises a tumor antigen (Ag);
a payload bioactive agent (PBA) covalently linked to the targeting bioactive agent (TBA) directly or indirectly;
a polymer; and
the ADC having a formula selected from Formula F1 through Formula F9, Formula F9a, Formulas F23-F30, disclosed herein, an isomer thereof, an isotope derivative thereof, and a combination thereof, wherein, m can be an integer in a range of from 2-100.

In some cases, the Nano-ADC can comprise the PBA comprising at least a compound having a formula selected from F30-F42, an isomer thereof, an isotope derivative thereof, and a combination thereof. In some cases, Nano-ADC can comprise a molar ratio of the TBA to the PBA in a range of 1:2 to 1:100. As mentioned above, it is understood by those skilled in the art that although n is an integer in a range of from ito 100, actual measurements of a TBA:PBA ratio can be a number in any of the aforementioned ranges including a fraction due to practical measurement limitations and/or the potential presence of some un-conjugated TBA, such as un-conjugated antibody. As disclosed above, the Nano-ADC can comprise a measured TBA:PBA in a range of from 1:0.01 to 1:100. In some cases, the Nano-ADC can comprise free antibody that is not conjugated with a payload bioactive agent (PBA).

In some cases, the Nano-ADC can comprise a mixture of TBA, wherein some of the TBA can be covalently linked to the PBA and some of the TBA can be non-covalently mixed with the PBA leading to more TBAs than the PBAs. In some cases, the Nano-ADC can comprise antibody molecules that are free from bound drugs, i.e., free antibody without conjugated to a drug molecule. In some cases, the Nano-ADC can comprise a molar ratio of TBA:PBA 100:1, 20:1, 10:1, 5:1, 4;1, 3;1, or 2:1 (on a reverse ratio of 1:0.01, 1:0.1, 1:0.2, 1:0.25, 1:0.3, and 1:0.5). In some cases, the molar ratio of antibody to a drug can be 100:1, 10:1, 5:1, 4;1, 3;1, or 2:1 (on a reverse ratio of 1:0.01, 1:0.1, 1:0.2, 1:0.25, 1:0.3, and 1:0.5).

In some cases, the antibody-drug conjugate (ADC) disclosed here can comprise a molar ratio of the TBA to the PBA in a range of 1:2 to 1:100. In some cases, the antibody-drug conjugate (ADC) disclosed here can comprise a molar ratio of an antibody to the PBA in a range of 1:2 to 1:100.

In some cases, this disclosure is directed to a method for treating or preventing a disease of a subject in need thereof, the method comprising administering the subject with an effective dose of the pharmaceutical compositions disclosed herein. Any of the pharmaceutical compositions disclosed herein or a combination thereof can be suitable.

Suitable to the method disclosed herein, the pharmaceutical composition can be administered to the subject via intravenous (IV) injection, subcutaneous (SC) injection, intramuscular (IM) injection, intradermal (ID) injection, oral administration, aerosol administration, or a combination thereof.

In some cases, the method can further comprise the step of administering the subject with one or more subsequent bioactive agents selected from a protein, a peptide, an antibody, a fragment of an antibody, a chemical compound, a small molecule drug, one or more chemotherapy drugs, a vaccine, and a combination thereof, wherein each of the one or more subsequent bioactive agents is administered to the subject, prior to, at the same time as, or after administering the pharmaceutical composition. In some cases, one or more subsequent bioactive agents can be the same as the TBA or the PBA. In some cases, one or more subsequent bioactive agents can be different from the TBA or the PBA.

In some cases, the disease can be selected from one or more immune disorders, infectious diseases, cancers, and a combination thereof.

The subject can be a human patient having a heterogenous tumor. The subject can be a human patient having multiple heterogenous tumors. In some cases, the subject can have tumor cells from the same type of tumors, different types of tumors, heterogenous tumors that comprise both Ag⁺ and Ag⁻ tumor cells, or a combination thereof.

In some cases, the subject can comprise Ag⁻ tumor cells that are adjacent to Ag⁺ tumor cells, such as in a same solid tumor or grown in the same media or cell culture space; distal to any Ag⁺ tumor cells, such as in a tumor or a location away from any Ag⁺ tumor cells; free from contact with any Ag⁺ tumor cells, such as in a separate tumor in a far distance part of the body of a subject, or grown in a separate media or cell culture space; or a combination thereof.

In some cases, the pharmaceutical composition can comprise a targeting bioactive agent (TBA) comprising an antibody or a part thereof having binding affinity to Her2, the Ag⁺ tumor cytotoxicity to HER2⁺ tumor cells, and the Ag tumor cytotoxicity to HER2⁻ tumor cells. In some cases, the HER2⁻ tumor cells are adjacent to the HER2⁺ tumor cells, distal to the HER2⁺ tumor cells, free from contact with any the HER2⁺ tumor cells, or a combination thereof. The HER2⁺ and HER2⁻ tumor cells can be adjacent to each other, such as within a solid tumor or solid tumors adjacent to each other, or distal to each other, such metastatic tumors. The HER2⁺ and HER2⁻ tumor cells can be in the same type of tumors, in different types of tumors, heterogenous tumors that comprise both Ag⁺ and Ag⁻ tumor cells.

In some cases, the subject can be a human patient having heterogenous HER2⁺ and HER2⁻ breast cancer, metastatic breast cancer, triple-negative breast cancer (TNBC), gastric cancer, colon cancer, or a combination thereof. In some cases, the subject can be a human patient selected from having heterogenous HER2⁺ and HER2⁻ breast cancer, metastatic breast cancer, triple-negative breast cancer (TNBC), gastric cancer, colon cancer, and a combination thereof.

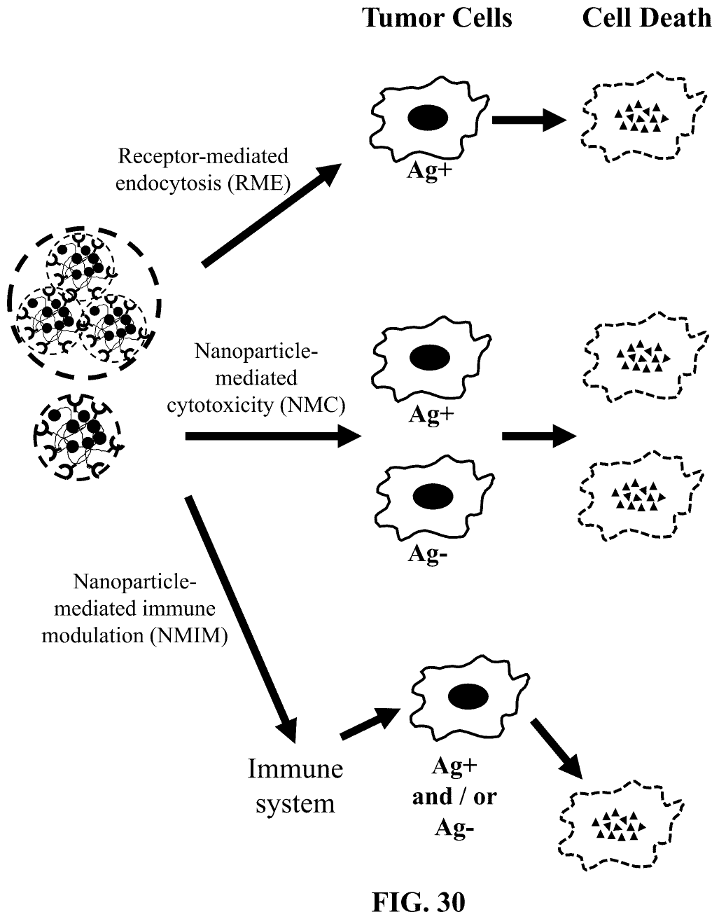

Not wishing to be bound by a particular mechanism or theory, Applicants believe that, in addition to improve water solubility of water insoluble payload (PBA) drugs, the nanocomposition of this disclosure including the ADC produced herein, referred to as “Nano-ADC” herein, provides hydrodynamic sizes that can be an important factor in the specificity of drug delivery, herein referred to as “nanoparticle-mediated cytotoxicity (NMC)” to tumor cells. A potential model of this mechanism is schematically illustrated in FIG. 30. It has been reported that accumulation of IV-injected nanoparticles in cancer tissues depends on the passive diffusion of these particles across the hyperpermeable tumor vasculature and retention in the interstitium via a slower clearance rate due to impaired lymphatics (Serpe, “Nanomaterials for Cancer Therapy”, Challa S and Kumar S R, eds, Wiley-VCH Verlag, Weinheim, p 1-39, 2006). This enables the encapsulated drug molecules to persist in situ for a longer time compared to non-encapsulated drugs. The basic mechanism for the specificity of drug delivery using nanoparticles can depend on the abnormality of blood vessels associated with tumors. Tumors typically contain abnormal vascularization wherein the blood vessels, unlike normal ones, have a discontinuous endothelium, with gaps between cells that may be several hundred nanometers large, creating sites of hyperpermeability in the tumor and allowing macromolecular transport to occur via these gaps (Jain 1987, Baban and Seymour 1998). Such nanoparticle penetrating effect was also observed in nano-chemotherapy drug FID-007 disclosed in U.S. Pat. No. 11,801,230, granted on Oct. 31, 2023 to Ray Yin, et al. Nanoparticles containing anti-tumor drugs such as FID-007 are designed to take advantage of these leaky channels or portals into the tumor mass, which openings usually do not exist in normal cells. The effect can lower the non-specific toxicity of the drug since its size reduces its ability to enter normal cells, while allowing the drug to accumulate in neoplastic tissue due to their hyperpermeable vasculature. So far, no such nanoparticle-mediated cytotoxicity has been reported with ADCs. The Nano-ADCs disclosed herein are configured to be within a size range that can be suitable for nanoparticle-mediated cytotoxicity to tumor cells. The Nano-ADCs disclosed herein can also comprise cleavable linkers that can deliver the payload cytotoxic drug once the Nano-ADC enters tumor cells. The TBA antibody can help to deliver the payload to the vicinity of those tumor cells.

An additional advantage of the pharmaceutical composition of this disclosure is that multiple molecules of a drug (payload bioactive agent, PBA) can be linked to the targeting bioactive agent (TBA). The polymer can be a branched polymer and can be linked to the TBA, such as a monoclonal antibody, at only a limited number of sites, such as at one cysteine or one lysine residue site, while at the same time linked to multiple molecules of the drug (see at least FIG. 2A-FIG. 2E, FIG. 3A-FIG. 3D, FIG. 4A-FIG. 4D, FIG. 5, FIG. 6, FIG. 7, FIG. 8, FIG. 9, FIG. 10A-FIG. 10B, and FIG. 11A-FIG. 11B). This can significantly improve the PBA to TBA ratio, such as drug to antibody ratio increasing payload of the ADC. In addition, the drugs (PBAs) can be positioned far away from the TBA, such as an antibody, the drugs can be more readily available due to less stereo hindrance.

Yet another advantage of the pharmaceutical composition of this disclosure is that the linkers L1 and L2 can be the same or different. By selecting different linkers L1 and L2, TBA and PBA can be selectively dissociated and selectively deliver the payload bioactive agent (PBA) to target cells. The term “drug” or “Drug” shown in drawings can refer to “PBA” or a specific drug.

The instant disclosure now will be exemplified in the following non-limiting examples.

### EXAMPLES

The present disclosure is further defined in the following Examples. It should be understood that these Examples, while indicating preferred embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various uses and conditions.

### MATERIALS AND MEASUREMENTS

### Nanoparticle Measurement

The size of various polymers, nanoaggregates, as well as antibody-polymer-drug nanoaggregates were measured by a dynamic light scattering (DLS) method using a Malvern Zetasizer Nano-ZS Zen3600 particle size analyzer (Malvern Panalytical Inc., Westborough, MA 01581, USA).

### In Vitro Cytotoxicity Measurements

Standard commercially available viability/cytotoxicity assay kit was used to assess cytotoxicity of the reagents with tumor and non-tumor cell lines as indicated.

### Example 1

### Synthesis of Mal-PEG₂₄-VA-PAB-Exatecan (Mal-PEG₂₄-VPE)

To a stirred solution of VPE (60.0 mg, 0.07 mmol) in DMF (2.0 mL), Mal-dPEG24-NHS ester (100.0 mg, 0.07 mmol) was added at 0° C., followed by addition of diisoproylethyl amine (0.01 mL, 0.07 mmol). After 30 minutes, the reaction mixture was warmed to room temperature and stirred for 30 minutes. The crude product was purified via Reverse Phase flash chromatography (C18 column). The desired product, herein “Mal-PEG₂₄-VPE”, was recovered as a yellow solid after lyophilization (45.0 mg, 0.022 mmol). The product was confirmed by H NMR and HPLC analysis.

### ADC Preparation:

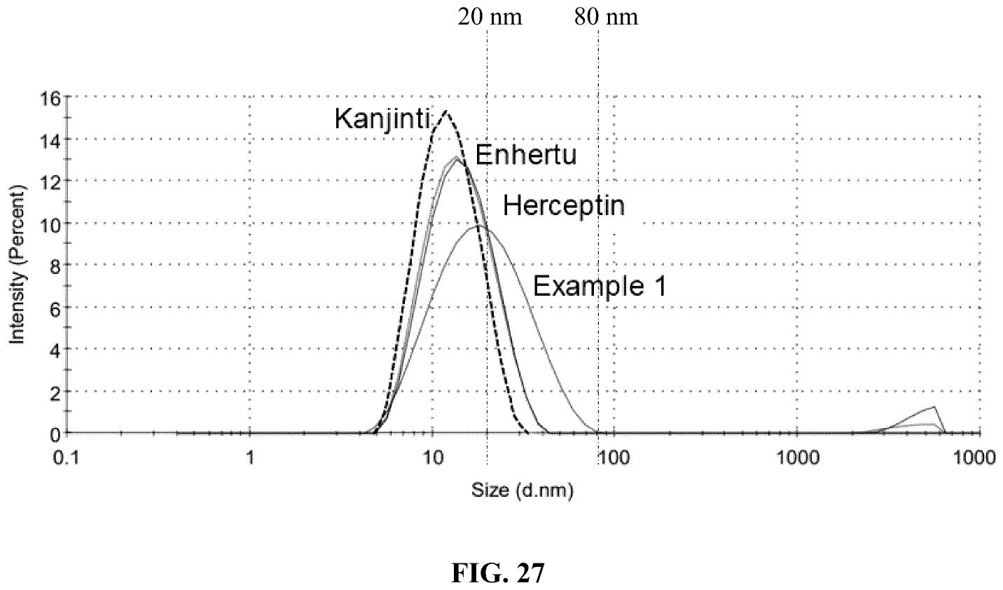

ADC comprising an anti-HER2 antibody Kanjinti, PEG polymer with 24 monomer units (m=24), cleavable peptide linker VAPAB and Exatecan conjugate was prepared as illustrated in FIG. 18B (Formula F11). Nanoparticle measurements are shown in FIG. 27. Herceptin: HERCEPTIN® (Trastuzumab), a HER2 monoclonal antibody. Enhertu: ENHERTU® (fam-trastuzumab-deruxtecan-nxki), HER2-deruxtecan ADC comprising monoclonal antibody Trastuzumab and cytotoxic drug deruxtecan. Kanjinti: Herceptin biosimilar known as a brand name KANJINTI® (trastuzumab-anns).

The VAPAB conjugation was prepared as described here: 21 nmol of an antibody was reacted with 210 nmol of TCEP (tris(2-carboxyethyl)phosphine) in PBS, pH 6.0 with 5 mM of EDTA, reacted at 37° C. for 1 hour, followed by the addition of 420 nmol of Mal-PEG₂₄-VPE prepared above, incubated at room temperature for 1 hour. The reaction was terminated with 840 nmol of N-Acetyl-L-cysteine at room temperature for 20 minutes. The mixture was purified with a gel filtration chromatography column and filtration. The conjugate collected was at about 50 kD molecular weight (MW) cut-off and was characterized with HIC, SEC and UV.

ADCs containing polymers having various PEG monomer units selected from PEG2, PEG4, PEG8, PEG12, PEG36 and PEG48, were prepared in a similar manner.

### Examples 2-6

Multiple ways can be employed to calculate the differences in cytotoxicity. One way to calculate a difference (in percentage number) of the Ag⁻ tumor cytotoxicity (Viability %: V_{AgNT}) v.s. the non-tumor cytotoxicity (Viability %: Vₙₒᵣₘₐₗ) was based on cell viability using the following formula:

$$difference = {V}_{AgNT .} - {V}_{normal} .$$

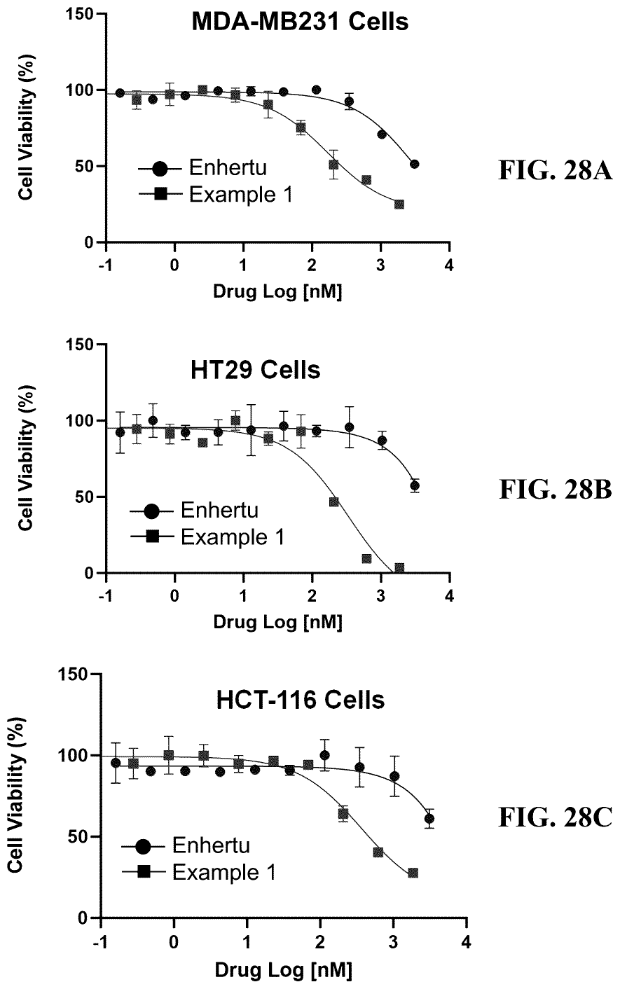

FIG. 28A-FIG. 28C show representative examples of cytotoxicity measured using in vitro cell assay. The Nano-ADC produced in EXAMPLE 1 (herein “Nano-ADC”) was used. (A) Cytotoxicity assay using MDA-MB231 cells, a triple-negative breast cancer (TNBC) cell line, i.e., HER2 Ag⁻ tumor cells). (B) Cytotoxicity assay using HT-29 cells, a colon cancer cell line, also HER2 Ag⁻ tumor cells. (C) Cytotoxicity assay using HCT-116 cells, a colon cancer cell line, HER2 Ag⁻ tumor cells. Enhertu: Commercial name for HER2-deruxtecan ADC comprising Trastuzumab and deruxtecan.

The Nano-ADC (from EXAMPLE 1) exhibited higher Ag tumor cytotoxicity of towards the HER2⁻ cells, than the cytotoxicity of the traditional anti-HER2 ADC, ENHERTU® towards the HER2⁻ cells, i.e., MDA-MB231 cells (FIG. 28A), or colon cancer cells (FIG. 28B and FIG. 28C), respectively, at all concentrations tested.

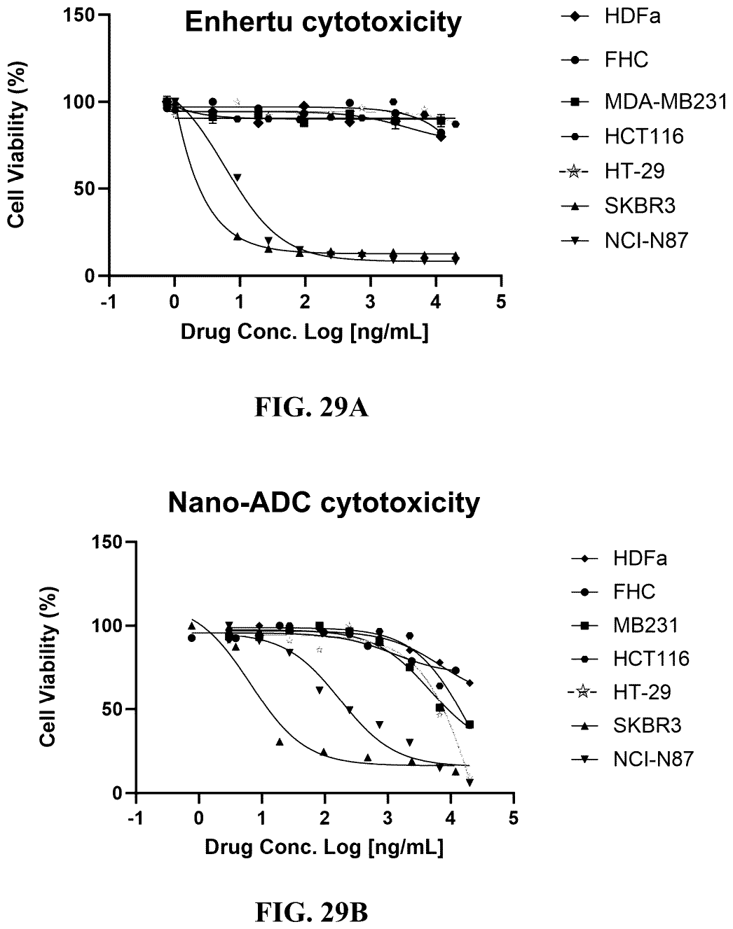

FIG. 29A-FIG. 29B show representative examples of cytotoxicity in non-tumor cells, Ag⁻ and Ag⁺ tumor cells: (A) Enhertu cytotoxicity; (B) Nan-ADC (EXAMPLE 1) cytotoxicity. HDFa: Primary Dermal Fibroblast cells from normal human. HFC: epithelial cells from normal human. SK-BR-3: a human breast cancer cell line that overexpresses the Her2 (Neu/ErbB-2) gene. NCI-N87: cells having high level of the Her2 (ERBB2) gene expression.

In the assay shown in FIG. 29A, ENHERTU® ADC exhibited no detectable cytotoxicity towards the non-tumor cells HDFa and FHC (Vₙₒᵣₘₐₗ) and the Ag tumor cells MDA-MB231, HT-29 and HCT-116 (V_{AgNT}), respectively, at corresponding concentrations throughout the entire range tested. ENHERTU® exhibited high cytotoxicity towards the HER2T cells SK-BR-3 and NCI-N87 (FIG. 29A).

The Nano-ADC of EXAMPLE 1 showed low cytotoxicity towards the non-tumor cells HDFa and FHC (Vₙₒᵣₘₐₗ) and cytotoxicity towards to the Ag⁻ tumor cells MDA-MB231, HT-29 and HCT-116 (V_{AgNT}), at low concentrations. At higher concentrations, Nano-ADC exhibited high Ag⁻ tumor cytotoxicity towards the Ag⁻ tumor cells MDA-MB231, HT-29 and HCT-116. The Ag⁻ tumor cytotoxicity (MDA-MB231, HT-29 and HCT-116 cells) of the Nano-ADC was at least 30% higher than the non-tumor cytotoxicity (non-tumor cells HDFa and FHC) at the concentrations indicated (FIG. 29B).

The Nano-ADC of EXAMPLE 1 exhibited high cytotoxicity towards the Ag⁺ tumor cells, i.e., HER2⁺ SK-BR-3 and NCI-N87 cells (FIG. 29B). Ag⁻ tumor cytotoxicity towards the Ag⁻ tumor cells MDA-MB231, HT-29 and HCT-116 was at least 30%, at least 40%, at various concentrations as indicated, respectively, of the Ag⁺ tumor cytotoxicity towards the Ag⁺ tumor cells SK-BR-3 and NCI-N87.

### Example 7

### Preparation of Boc-VAPAB-Exatecan:

The Exatecan Mesylate solution in DMF was prepared as following: DMF (3.0 mL) was added to Exatecan Mesylate (476.0 mg, 0.90 mmol) in a sealed vial, followed by addition of diisopropylethylamine (0.02 mL, 0.1 mmol).

To a stirred solution of Boc-Val-Ala-PAB-PNP (0.50 g, 0.90 mmol) in DMF (10.0 mL) was added the Exatecan solution prepared above, followed by diisopropylethyl amine (0.16 mL, 0.90 mmol) dropwise at 0° C. After 30 minutes, it was warmed to room temperature and stirred for 12 hours. The crude product was purified via Combi-flash column, using a gradient of 50-100% ethyl acetate in hexanes) to afford the desired product as yellow solid (566.0 mg, 0.60 mmol). The product was confirmed by H NMR and HPLC.

### Example 8

### Preparation of VAPAB-Exatecan:

To a stirred solution of Boc-Val-Ala-PNP-Exatecan (566.0 mg, 0.60 mmol) in CH₂Cl₂ (4.0 mL) was added TFA solution (6.0 mL, 1:1 TFA/CH₂Cl₂). After 1 hour, the solvent was removed in vacuo. The crude product was purified via Reverse Phase flash chromatography (C18 column) to afford the desired product as yellow solid (150.0 mg, 0.18 mmol).

### Example 9

### Synthesis of Low Molecular Weight EOx Oligomers and Polymers:

Following ingredients were added into a 40 ml glass vial with a magnetic stir bar: 3.6 g methyl bromoacetate initiator, 2.3 ml 2-ethyl-2-oxazoline (EOx stored in 3 A molecular sieves) and 7.5 ml anhydrous acetonitrile, all under the blanketing of nitrogen. The vial was heated for about 24 hours at 80° C. After cooling to room temperature, a mixture of water and methanol at 1:15 ratio were added to terminate the EOx polymerization. The EOx oligomers were precipitated out of the solution by slowly mixing into a beaker with well agitated methyl-tert-butyl-ether (MTBE). The oligomer crude product was recovered after the liquid decanting and vacuum drying. The oligomer product was hydrolysed to produce the desired —OH ends and purified by dialysis with MWCO (molecular weight cut-off) of 100-500 D and dried. The H-NMR confirmed the structure as HO₂C—CH₂—(N(COCH₂CH₃)CH₂CH₂)ₙOH and contained monomers, dimers, trimers, tetramers and pentamers, n=1-5, with a calculated number average molecular weight Mn of 302 g/mol and weight average molecular weight Mw of 339 g/mol, i.e., a polydispersity of 1.12.

PEOX Polymers (HO₂C—CH₂—(N(COCH₂CH₃)CH₂CH₂)ₙOH) with n=1-100 were prepared in a similar manner.

### Example 10

PEOX oligomer exatecan conjugation as following:

To a stirred solution of EDC-HCl (56.0 mg, 0.29 mmol) in DMF (3.0 mL) was added OxymaPure (42.0 mg, 0.29 mmol) and EOx oligomer (80.0 mg, 0.29 mmol) prepared above. Exatecan Mesylate (154.0 mg, 0.29 mmol) was added to the mixture solution followed by the addition of diisopropylethylamine (0.05 mL, 0.29 mmol). The reaction mixture was stirred at 0° C. for 1 hour, then it was warmed to room temperature and stirred for 12 hours. The crude product was purified as white solid (14.5 mg, 0.02 mmol). The HPLC analysis indicated the PEOx oligomer exatecan conjugation consisted of monomer, dimer, trimer, tetramer.

### Example 11

PEOX exatecan intermediate was synthesized with the reaction scheme shown in FIG. 7.

### Example 12

An ADC comprising PEOX exatecan was synthesized with the reaction scheme shown in FIG. 8 with an —N— linkage via a lysine.

### Example 13

An ADC comprising PEOX exatecan was synthesized with the reaction scheme shown in FIG. 9 with an —S— linkage via a cysteine.

What is claimed is:

1. A pharmaceutical composition comprising:
a targeting bioactive agent (TBA) comprising at least a targeting moiety having binding affinity to a target molecule or a target cell;
a payload bioactive agent (PBA) covalently linked to said TBA directly or indirectly; and
a polymer comprising at least one first terminal group and at least one second terminal group; and optionally a pharmaceutically suitable carrier;
wherein said polymer is covalently or non-covalently linked to said TBA, said PBA, or a combination thereof,
wherein said polymer comprises, polyoxazoline (POX), a polyethylene glycol (PEG), or a combination thereof,
wherein said POX comprises a linear portion, a branched portion, or a combination thereof, and
wherein said POX comprises poly(2-methyloxazoline), poly(2-ethyloxazoline), poly(2-propyloxazoline), poly(2-isopropyloxazoline), poly(2-substituted oxazoline), or a combination thereof.

2. The pharmaceutical composition of claim 1, wherein said polymer is covalently linked to said TBA and covalently linked to said PBA, wherein said polymer comprises at least one reacted first terminal group and at least one reacted second terminal group; and
wherein said polymer is positioned between said TBA and said PBA with at least one of said reacted first terminal group covalently linked to said TBA and at least one of said reacted second terminal group covalently linked to said PBA.

3. The pharmaceutical composition of claim 2, wherein said polymer comprises the PEG covalently linked to said TBA at the at least one reacted first terminal group and covalently linked to said PBA at the at least one reacted second terminal group, wherein said PEG comprises PEG monomer units in a range of from 1 to 100.

4. The pharmaceutical composition of claim 2, wherein said polymer comprises said POX covalently linked to said TBA at the at least one reacted first terminal group and covalently linked to said PBA at the at least one reacted second terminal group, said POX comprises poly(2-methyloxazoline), poly(2-ethyloxazoline), poly(2-propyloxazoline), poly(isopropyloxazoline), or a combination thereof.

5. The pharmaceutical composition of claim 2, further comprising a linker L1, a linker L2, or a combination thereof,
wherein said linker L1 is covalently linked to said TBA and covalently linked to said reacted first terminal group of said polymer, said linker L2 is covalently linked to said PBA and covalently linked to said reacted second terminal group of said polymer, and at least one of said linker L1 and said linker L2 comprises a cleavable linker that is cleavable in vivo or in said target cell.

6. The pharmaceutical composition of claim 5, wherein said cleavable linker comprises an enzymatically-cleavable peptide linker, an acid sensitive hydrazone linker, a linker comprising disulfides, a disulfide linker, a thioether linker, a glutathione-sensitive disulfide linker, a linker containing a sugar molecule or moiety, a glucuronide-containing linker, a glycosidase-cleavable linker, a phosphatase-cleavable linker, an esterase-cleavable linker, a hydrolysis-cleavable linker, a monodispersed PEG linker, a PEO linker, a poly-PEG linker, a GGFG linker, a VAPAB linker, a dipeptide linker, a valine-citrulline linker, a valine-alanine linker, a polypeptide linker, a p-aminobenzyloxycarbonyl (PABC) linker, or a combination thereof.

7. The pharmaceutical composition of claim 1, wherein said polymer comprises said POX that is non-covalently associated with said TBA and said PBA forming nanoaggregates,
wherein said POX comprises poly(2-methyloxazoline), poly(2-ethyloxazoline), poly(2-propyloxazoline), poly(isopropyloxazoline), or a combination thereof, and
wherein said POX comprises the at least one first terminal group modified with H or a hydrophobic moiety and the at least one second terminal group modified with a hydrophilic moiety,
wherein said first terminal group comprises in a range of from 1% to 99% of H and 1% to 99% of said hydrophobic moiety that comprises saturated or unsaturated aliphatic hydrocarbons having about 1 to about 22 carbons, an aromatic hydrocarbon, or a combination thereof, and said second terminal group comprises a group modified by an amine, amide, imine, imide, carboxyl, hydroxyl, ester, ether, acetate, phosphate, ketone, aldehyde, sulfonate, or a combination thereof.

8. The pharmaceutical composition of claim 7, wherein a range of from 1% to 100% of said second terminal group is free from primary amine.

9. The pharmaceutical composition of claim 7, wherein a range of from 1% to 100% of said second terminal group comprises hydroxyl group.

10. The pharmaceutical composition of claim 1, further comprising a polymer side chain that is covalently linked to said TBA, said polymer, or a combination thereof,
wherein said polymer side chain comprises POX comprising poly(2-methyloxazoline), poly(2-ethyloxazoline), poly(2-propyloxazoline), poly(isopropyloxazoline), or a combination thereof.

11. The pharmaceutical composition of claim 1, wherein said pharmaceutical composition comprises a tumor cytotoxicity to tumor cells and a non-tumor cytotoxicity to non-tumor cells, wherein said tumor cytotoxicity comprises Ag+ tumor cytotoxicity to tumor cells having Ag+ tumor cells and Ag− tumor cytotoxicity to tumor cells free from Ag− tumor cells.

12. The pharmaceutical composition of claim 11, wherein said pharmaceutical composition comprises said Ag− tumor cytotoxicity to said Ag⁻ tumor cells in absence of said Ag⁺ tumor cells.

13. The pharmaceutical composition of claim 12, wherein said Ag⁻ tumor cytotoxicity is at least 20% higher than said non-tumor cytotoxicity.

14. The pharmaceutical composition of claim 12, wherein said Ag− tumor cytotoxicity, Ag+ tumor cytotoxicity, and non-tumor cytotoxicity, each is measured in a cell-based assay.

15. The pharmaceutical composition of claim 1, wherein said TBA comprises a binding affinity to a target molecule selected from a gene or a gene product of PD-1, PD-L1, 4-1BB, 5T4 (5T4 oncotrophoblast glycoprotein), ABL1, ABL2, ACVR1, AKR1C3, AKT, ALK, ASCL1, ASNS, Asparagine, ASGR1, ASGPR1, ATM, ATR, ATRX, AURKA, AURKB, AXL, B7H3 (CD276 antigen), B7H4 (V-set domain-containing T-cell activation inhibitor 1), B7H7 (HHLA2), BAK1, BAX, BCL2 family members (BCL2, BCL2L1, MCL1, BCL2A1, BAK1, BAX), BCL2A1 (BFL1), BCMA (TNFRSF17), BCOR, BET bromodomain family, BIRC5 (survivin), BMPR, BRAF, BRD1, BRD4, CCND1, CCND2, CCR8, CD19, CD22, CD25, CD274 (PD-L1), CD276 (B7-H3), CD3, CD16a, CD28, CD33, CD37, CD38, CD40, CD40L, CD47, CD52, CD7, CD70, CD73, CD79B, CD200R1 (Cell surface glycoprotein OX2 receptor 1), CDC7, CDK12, CDK2, CDK4, CDK6, CDK7, CDK9, CHEK1 (CHK1), CK1 (casein Kinase 1), CK2 (casein kinase 2), CLDN18.2, CRBN [cereblon E3 ubiquitin ligase], CREBBP/EP300, CRTAM, CSF1R, CSF2 (GM-CSF), CTAGlB (NY-ESO-1), CTLA4, CTNNB1, CXCL10, CXCR4, DDX3X, DLK1, DLL3 (Delta-like Ligand 3), DNA (alkylators), DNA-PK, DNMT (DNA methyl transferase), DOT1L, EED, EGFR, EGFRvIII (EGFR variant III), EpCAM (TROP1), EPHA2, ERBB2 (HER2), ETS gene fusions, EWSR1-FLI1, EZH2, F3 (tissue factor), FGFR, FLT3, FOLR1 (folate receptor alpha), Gamma secretase, GD2 (disialoganglioside), GFI1, GFI1B, GPC2, GPC3, GPNMB, GSK3, HAVCR2 (TIM3), TIMD4 (TIM4), HDAC, HIF1A, Hippo pathway (YAP1, WWTR1 (TAZ), TEADs), Histone H3, HSP90, IDH1, IDH2, IDO1, IFNG (interferon gamma), IGF1R, IL13RA2, IL2, IL3RA (CD123), IL6, IL23p19, Inhibitor of apoptosis (IAP) proteins, JAK1, JAK2, JAK3, KAT6A (MYST3), KDM1A (LSD1), KDM4A, KIT, KMT2A (MLL), KMT2E (MLL5), LAG3, LIFR, LIN28B, LRRC15, MAGEA3, MAP2K1 (MEK1), MAP2K2 (MEK2), MAPK3 (ERK1), MAPK1 (ERK2), MCL1, MDM2, MEN1 (menin), MET, MGMT, MS4A1 (CD20), MSLN (mesothelin), MTOR, mTORC1, mTORC2, MYC, MYCN, NAMPT, NCAM1 (CD56), MUC1, MUC16, NEDD8 activating enzyme (NAE), Neoantigens, NF-kappa-B, NKp30, NKp46, NOTCHI, NR5A1 (steroidogenic factor 1), NT5C2, NTRK, NUTM1 gene fusions, ODC1, OLIG2, PARP, PAX5, PAX-FOXO1, PDCD1 (PD-1), PD-L1, PDGFRA, PDGFRB, PDPK1 (3-phosphoinositide dependent protein kinase 1), PIK3CA (PI3K-alpha), PIK3CD (PI3K-delta), PIM1, PKA (protein kinase A), PKC (protein kinase C), PLK1, PML-RARA, PPM1D (WIP1), PRAME, PRDM1, PRDM10, PRDM8, PRMT2, PRMT5, Proteasome, PSMA, PTEN, PTK2 (FAK), PTPN (protein tyrosine phosphatase), PTPN11 (SHP2), RAS, RELA, RET, RIGI (DDX58), RNA polymerase (RNApol) I, ROR1, ROR2, ROS1, RPA3, SARS-Cov-2, SH2B3, SLC16A1 (MCT1), SMO, SMYD3, SSTR (somatostatin receptor), STAT2, STAT3, STEAP1, STING1 (STING), SUZ12, SWI/SNF, SYK, SYT-SSX, TERT, TET2, TGF-beta, TNFRSF4 (OX40), OX40L, TNFRSF8 (CD30), TNFSF10 (TRAIL), TOP1, TOP2 (DNA topoisomerase I/II), TROP2, TP53, TSLP, Tubulin, TYK2, TYMS, VEGF, VEGFR, WDR5, WEE1, WT1, XPO1 (Exportin 1), YAP1, ZBTB17 (MIZ-1), or a combination thereof.

16. The pharmaceutical composition of claim 1, wherein said TBA is selected from a peptide, a protein, DNA, RNA, nucleic acids, oligo nucleic acids, mRNA, siRNA, sgRNA, a monoclonal antibody, a fragment of a monoclonal antibody, a polyclonal antibody, a fragment of a polyclonal antibody, a synthetic antibody, a fragment of a synthetic antibody, antigen-binding portions thereof, an agonist, an activator, an inhibitor, an antagonist, a ligand of cell surface receptor, a naked nucleic acid, a nucleic acid, a ribozyme, a virus, a virus-like particles and a combination thereof, Alemtuzumab, alemtuzumab, an antisense nucleic acid, anti-CD19 antibodies, anti-CD20 antibodies, anti-CTLA-4 (cytotoxic T lymphocyte-associated antigen) antibodies, anti-LAG3 (lymphocyte activation gene-3) antibodies, anti-PD1 antibodies pembrolizumab, anti-PD-L1 antibodies, anti-TIM-3 (T cell immunoglobulin and mucin domain-3) antibodies, Atezolizumab, avelumab, Bevacizumab, bevacizumab, Blinatumomab, Brentuximab, Cetuximab, cetuximab, chimeric antigen receptor or chimeric antigen T-cell receptor (CAR-T), cytokines, Daratumumab, Denosumab, Dinutuximab, Durvalumab, Elotuzumab, gemtuzumab, granulocyte colony stimulating factor (G-CSF), Ibritumomab, durvalumab, Inotuzumab, interferon α, interferon α2a, interleukins, Ipilimumab, lectins, Necitumumab, Neupogen (Filgrastim), Obinutuzumab, Ofatumumab, Olaratumab, nivolumab, Panitumumab, Pembrolizumab, Pertuzumab, Ramucirumab, Rituximab, rituximab, Siltuximab, Tislelizumab (BGB-A317), Toripalimab, siRNAs, T-cell receptor (TCR), atezolizumab, tositumomab, trastuzumab, anti-EGFR, anti-PSMA, a member of a small molecule binding pair, and a combination thereof.

17. The pharmaceutical composition of claim 15, wherein said pharmaceutical composition comprises said Ag+ tumor cytotoxicity to HER2+ tumor cells and said Ag− tumor cytotoxicity to HER2− tumor cells, wherein said Ag− tumor cytotoxicity is measured in a cell-based assay with said HER2− tumor cells in the absence of said HER2+ tumor cells.

18. The pharmaceutical composition of claim 1, wherein said PBA comprises a natural or synthetic small molecule-based drug, inorganic-based drug, a toxin molecule having cytotoxicity, a molecule having cytotoxicity to said target cell, a derivative thereof, or a combination thereof.

19. The pharmaceutical composition of claim 18, wherein said PBA comprises 5-fluorouracil (5-FU), alkylators, anti-metabolites, cabazitaxel, Calicheamicin, Calicheamicin derivatives, camptothecin, camptothecin derivatives, capecitabine, cell division inhibitors or microtubule inhibitors, ciprofloxaxin, cyclophosphamide, deruxtecan, deruxtecan derivatives, DNA cleavage agent, DNA crosslinking agent, DNA replicating inhibitor, docetaxel, dolastatin analogs, doxorubicin, duocarmycin analogs, etoposide, everolimus, exatecan, exatecan derivatives, topoisomerase inhibitors, gemcitabine, irinotecan (CPT-11), larotaxel, maytansinoids (DM1, DM4), milataxel, MMAE (monomethyl auristatin E), MMAF (monomethyl auristatin F), MMAH (monomethyl auristatin H), mTOR inhibitor, one or more ATP-competitive mTORC1/2 inhibitors, one or more dual PI3K-mTOR inhibitors, ortataxel, ozogamicin, paclitaxel, pemetrexed, pyrrolobenzodiazepine dimer (PBD), rapamycin, ridaforolimus, SN-38, taxane, temsirolimus, tesetaxel, Topoisomerase 1 (Top 1) inhibitor, Topoisomerase 2 (Top 2) inhibitor, topotecan, torin-1, torin-2, vinblastine, vinca alkaloids, vincristine, vinorelbine, vistusertib, zotarolimus, biological toxins, ricin, or a combination thereof.

20. The pharmaceutical composition of claim 1, wherein said pharmaceutical composition is a drug for treating or preventing a disease selected from one or more immune disorders, infectious diseases, cancers, and a combination thereof.

21. The pharmaceutical composition of claim 1, wherein said pharmaceutical composition comprises a molar ratio of said TBA to said PBA in a range of from about 1:0.01 to about 1:100.

22. The pharmaceutical composition of claim 1, wherein said pharmaceutical composition comprises nanoaggregates having a size less than 120 nm before lyophilization, wherein said nanoaggregates comprise said TBA, said PBA, and said polymer.

23. The pharmaceutical composition of claim 1, wherein said pharmaceutical composition comprises an antibody-drug conjugate (ADC) having a formula selected from Formula F2 to Formula F27: 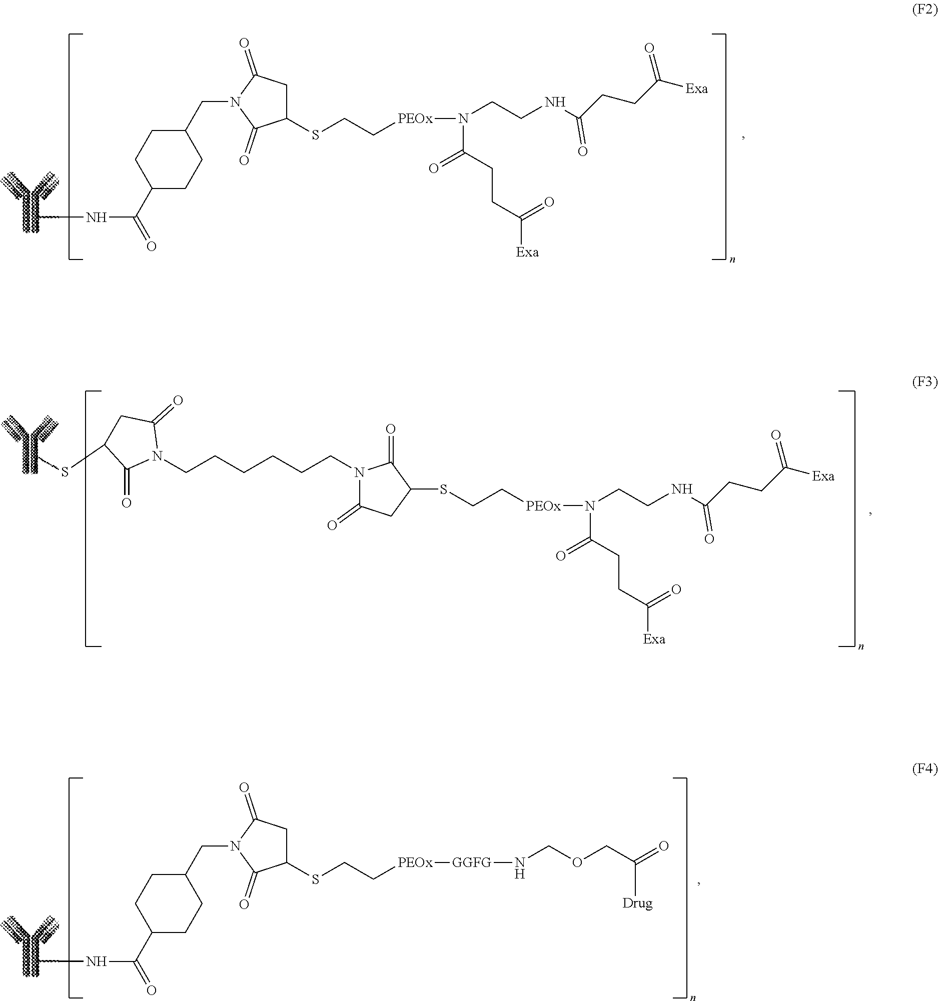 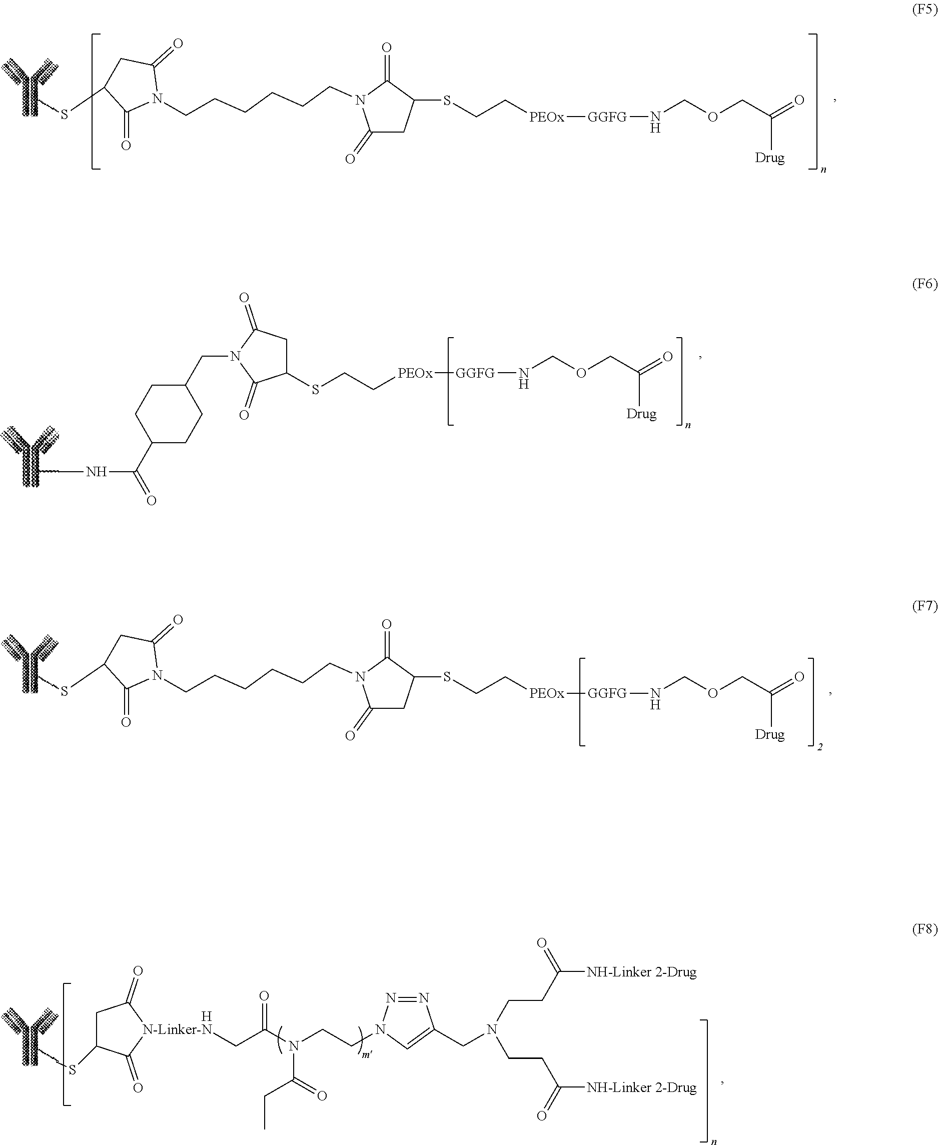 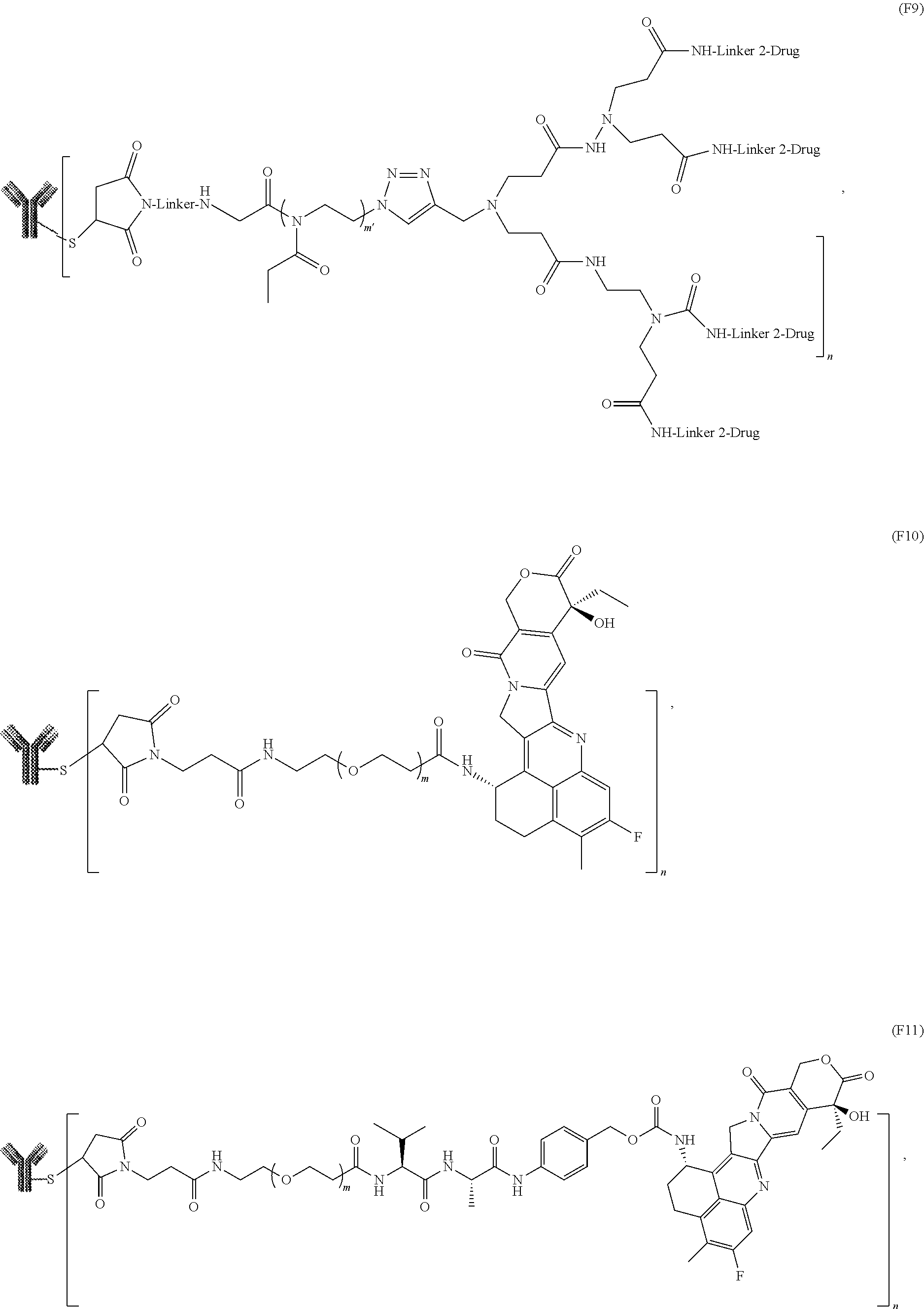 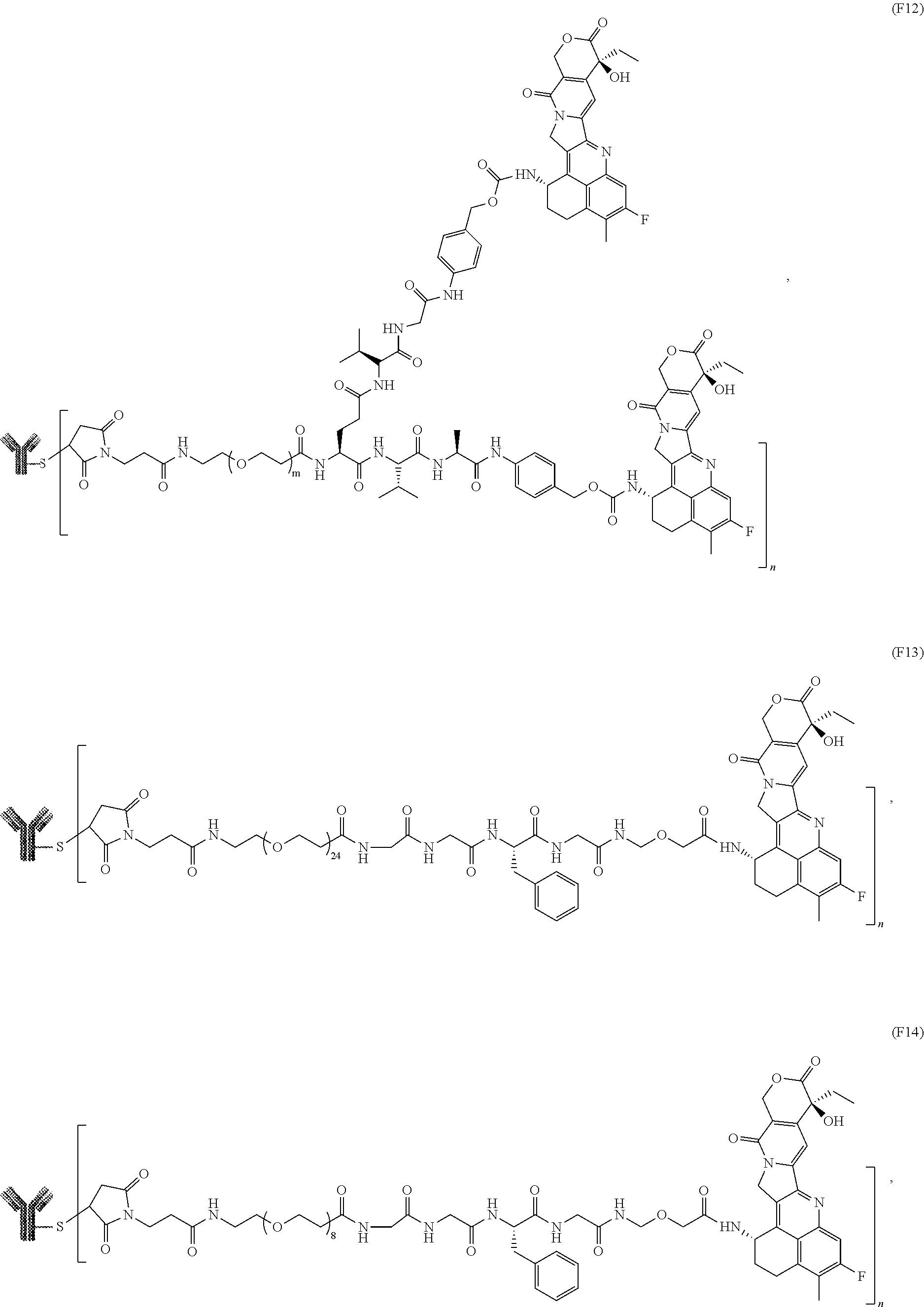 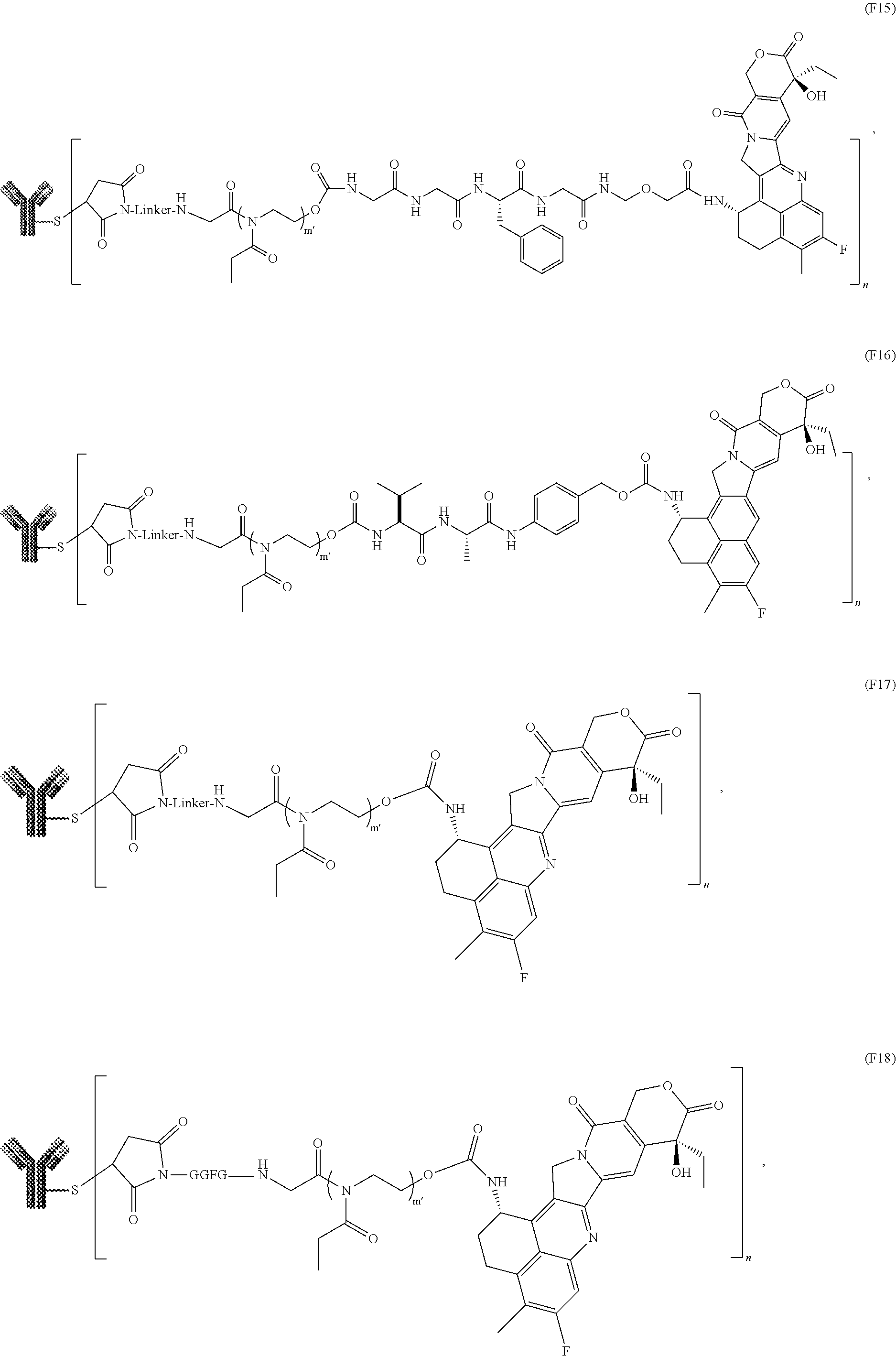 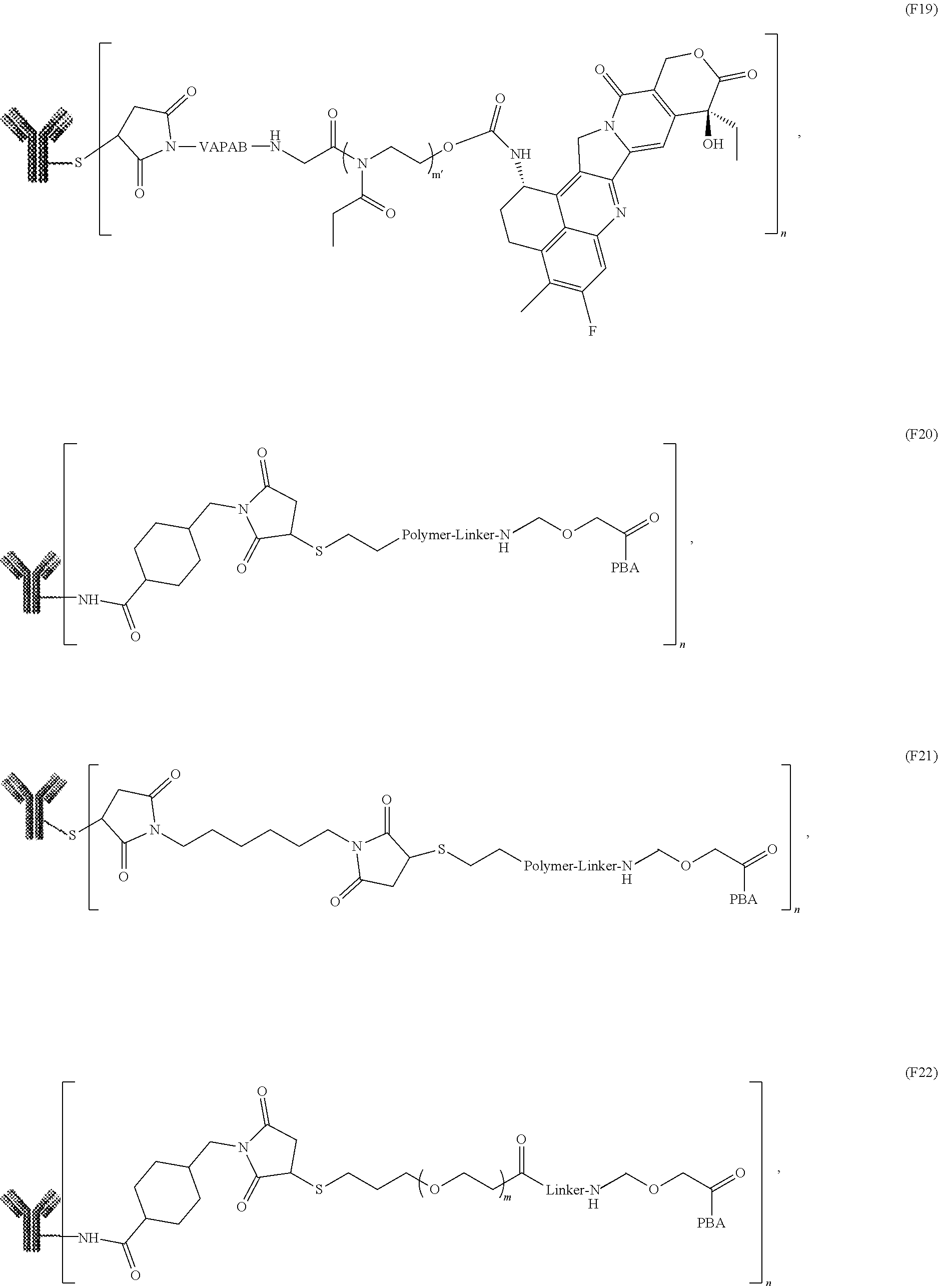 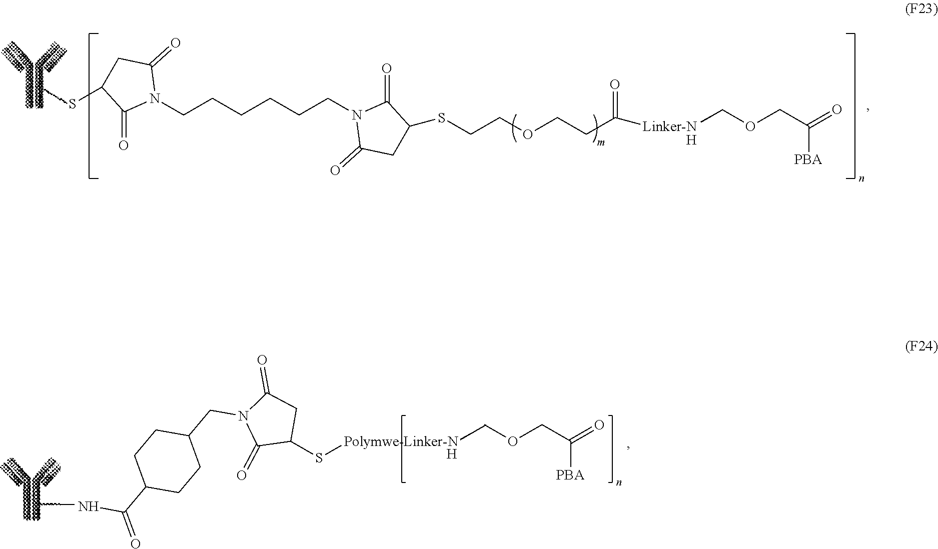 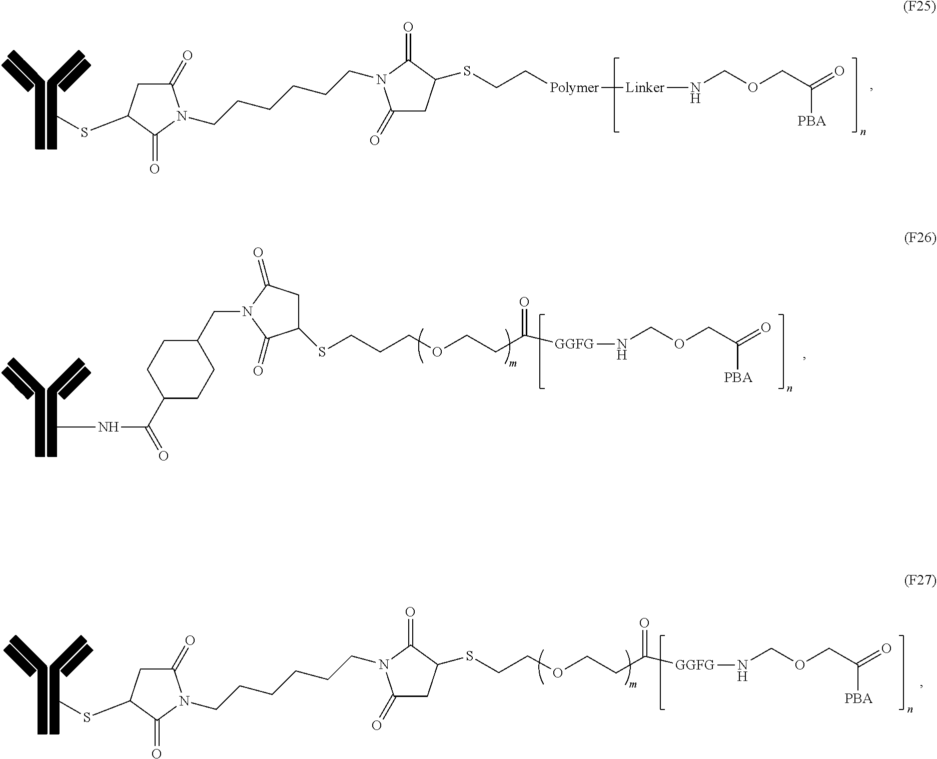
an isomer thereof, an isotope derivative thereof, and a combination thereof,
wherein, m is an integer in a range of from 1-100, m′ is an integer in a range of from 1-100, and n is an integer in a range of from 1 to 100.

24. The pharmaceutical composition of claim 1, wherein said PBA comprises at least a compound having a formula selected from Formula F30 to Formula F42: 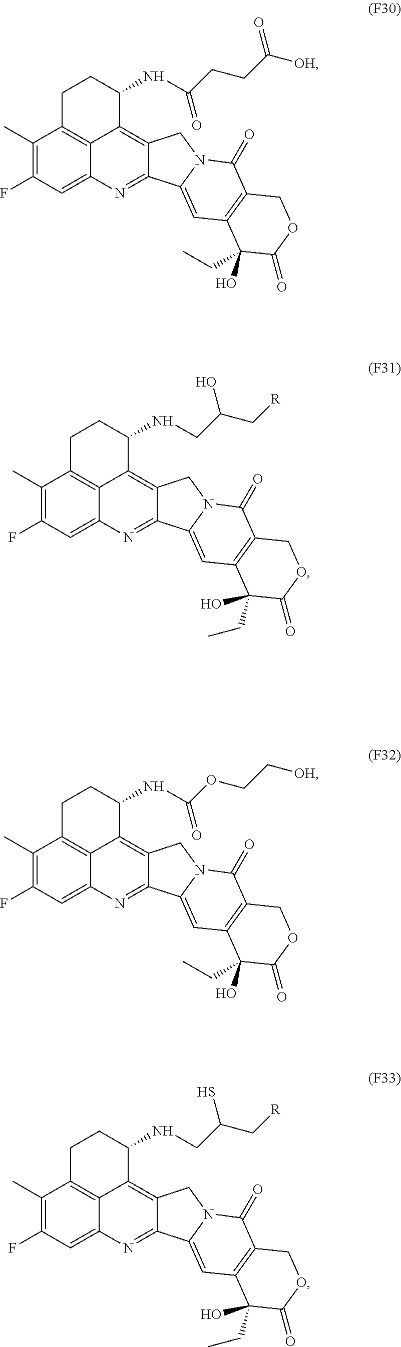 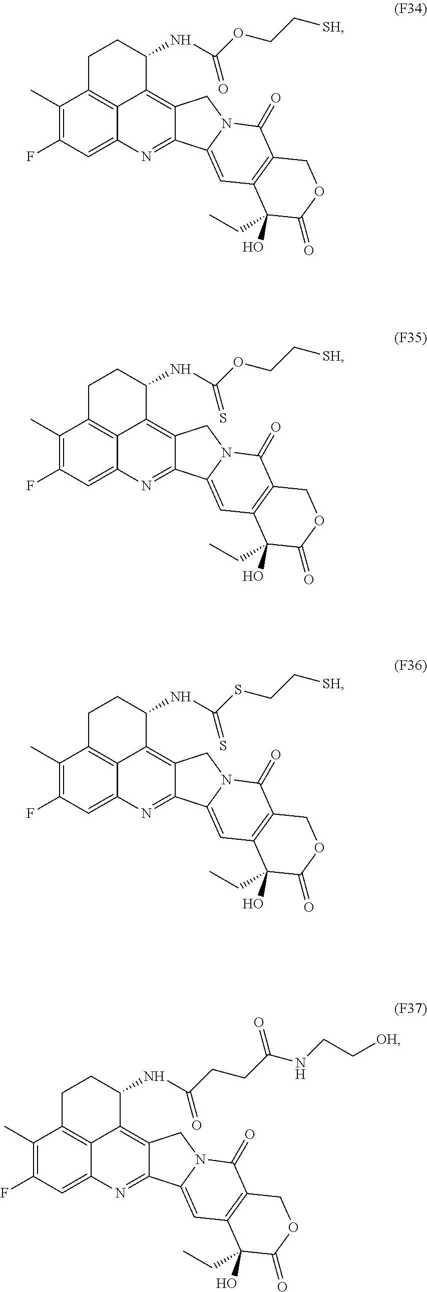 [SMILES: CC[C@@]1(O)C(=O)OCc2c1cc1n(c2=O)Cc2c-1nc1cc(F)c(C)c3c1c2[C@@H](NC(=O)OCCS)CC3 ; CC[C@@]1(O)C(=O)OCc2c1cc1n(c2=O)Cc2c-1nc1cc(F)c(C)c3c1c2[C@@H](NC(=S)OCCS)CC3 ; CC[C@@]1(O)C(=O)OCc2c1cc1n(c2=O)Cc2c-1nc1cc(F)c(C)c3c1c2[C@@H](NC(=S)SCCS)CC3 ; CC[C@@]1(O)C(=O)OCc2c1cc1n(c2=O)Cc2c-1nc1cc(F)c(C)c3c1c2[C@@H](NC(=O)CCC(=O)NCCO)CC3] 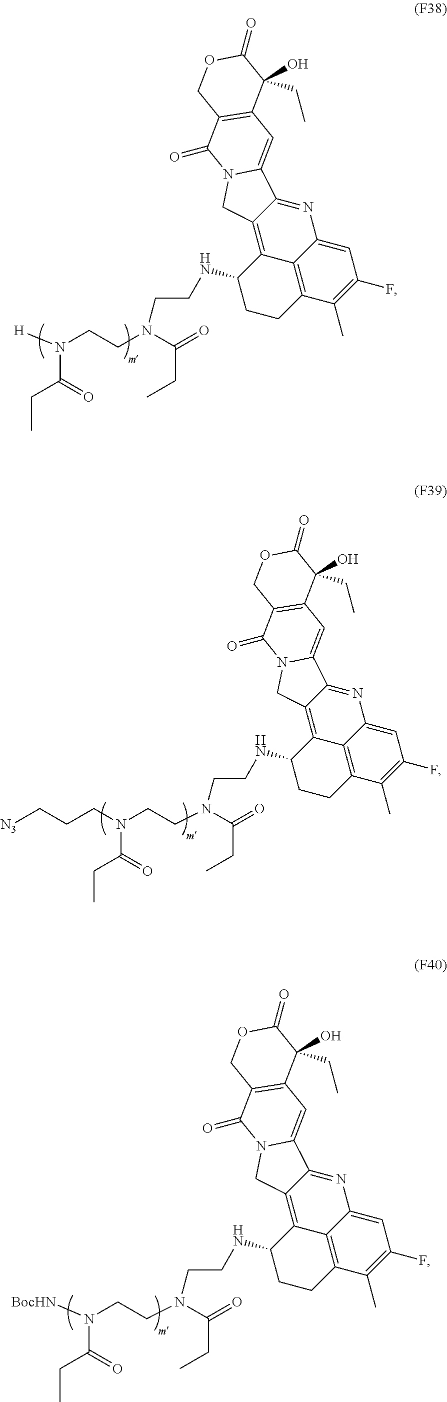 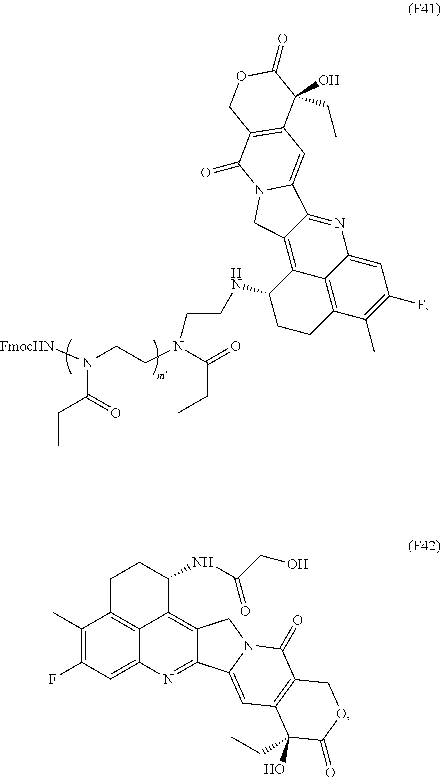
an isomer thereof, an isotope derivative thereof, and a combination thereof; and
wherein each said PBA is in a reacted form covalently linked to said TBA, said polymer, or said linker, m′ is an integer in a range of from 1 to 100.

25. An antibody-drug conjugate (ADC) comprising:
a targeting bioactive agent (TBA) comprising an antibody or a part thereof having a binding affinity to a target molecule that comprises a tumor antigen (Ag);
a payload bioactive agent (PBA), an isomer thereof, an isotope derivative thereof, or a combination thereof, covalently linked to said TBA directly or indirectly; and
a polymer;
wherein said ADC comprises a formula selected from Formula F2 to Formula F27, and a combination thereof,
wherein, m is an integer in a range of from 1 to 100, m′ is an integer in a range of from 1-100, and n is an integer in a range of from 1 to 100.

26. A method for treating or preventing a disease of a subject in need thereof, said method comprising administering to said subject an effective dose of said pharmaceutical composition of claim 1.
